SEQUENCE LISTING (126 sequences)

SEQ ID NO: 1 - protein, 459 aa, Homo sapiens [sequence omitted]

SEQ ID NO: 2 - protein, 117 aa, Mus musculus
  Murine Ab variable region sequence
QVQLQESGPGLVAPSQSLSITCTVSGFSLSRYNVHWVRQPPGKGLEWLGMIWDGGSTDYNSALKSRLSITKDNSKSQVFLKMNSLQTDDTAMYYCARNRYESGMDYWGQGTTVTVSS

SEQ ID NO: 3 - protein, 114 aa, Mus musculus
DIVMTQTPSSLTVTAGEKVTMSCKSSQSLLNSGNRKNYLTWYQQKPGQSPKLLIYWASTRESGVPDRFTGSGSGTDFTLIISSVQAEDLAVYYCQNDYTYPFTFGSGTKLEIKR

SEQ ID NO: 4 - protein, 5 aa, Mus musculus
RYNVH

SEQ ID NO: 5 - protein, 16 aa, Mus musculus
MIWDGGSTDYNSALKS

SEQ ID NO: 6 - protein, 6 aa, Mus musculus
NRYESG

SEQ ID NO: 7 - protein, 17 aa, Mus musculus
KSSQSLLNSGNRKNYLT

SEQ ID NO: 8 - protein, 7 aa, Mus musculus
WASTRES

SEQ ID NO: 9 - protein, 12 aa, Mus musculus
QNDYTYPFTFGS

SEQ ID NO: 10 - protein, 17 aa, Mus musculus
PARGESNWTHVSLFHTR

SEQ ID NO: 11 - protein, 16 aa, Mus musculus
VKCLTLNKLQDIYFIK

SEQ ID NO: 12 - protein, 30 aa, Mus musculus
QVQLQESGPGLVAPSQSLSITCTVSGFSLS

SEQ ID NO: 13 - protein, 14 aa, Mus musculus
WVRQPPGKGLEWLG

SEQ ID NO: 14 - protein, 32 aa, Mus musculus
RLSITKDNSKSQVFLKMNSLQTDDTAMYYCAR

SEQ ID NO: 15 - protein, 14 aa, Mus musculus
MDYWGQGTTVTVSS

SEQ ID NO: 16 - protein, 23 aa, Mus musculus
DIVMTQTPSSLTVTAGEKVTMSC

SEQ ID NO: 17 - protein, 15 aa, Mus musculus
WYQQKPGQSPKLLIY

SEQ ID NO: 18 - protein, 32 aa, Mus musculus
GVPDRFTGSGSGTDFTLIISSVQAEDLAVYYC

SEQ ID NO: 19 - protein, 8 aa, Mus musculus
GTKLEIKR

SEQ ID NO: 20 - protein, 15 aa, Homo sapiens
LDDYSFSCYSQLEVN

SEQ ID NO: 21 - protein, 22 aa, Homo sapiens
NFRKLQEIYFIETKKFLLIGKS

SEQ ID NO: 22 - protein, 10 aa, Homo sapiens
QEKDENKWTH

SEQ ID NO: 23 - protein, 15 aa, Homo sapiens
VKCLNFRKLQEIYFI

SEQ ID NO: 24 - protein, 15 aa, Homo sapiens
ETKKFLLIGKSNICV

SEQ ID NO: 25 - protein, 15 aa, Homo sapiens
RQEKDENKWTHVNLS

SEQ ID NO: 26 - protein, 15 aa, Homo sapiens
LDDYSFSCYSQLEVN

SEQ ID NO: 27 - protein, 22 aa, Homo sapiens
NFRKLQEIYFIETKKFLLIGKS

SEQ ID NO: 28 - protein, 42 aa, Homo sapiens
YREGANDFVVTFNTSHLQKKYVKVLMHDVAYRQEKDENKWTH

SEQ ID NO: 29 - protein, 119 aa, Mus musculus
EVKLLESGGGLVQPGGSLKLSCAASGFAFSAYWMSWVRQAPGKGLEWIGEINPDSSTINCTPSLKDKFIISRDNAKNTLSLQMNKVRSEDTALYYCARRLRPFWYFDVWGAGTTVTVSS

SEQ ID NO: 30 - protein, 112 aa, Mus musculus
DVLMTQTPLSLPVSLGDQASISCRSSQSIVQSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 31 - protein, 5 aa, Mus musculus
AYWMS

SEQ ID NO: 32 - protein, 17 aa, Mus musculus
EINPDSSTINCTPSLKD

SEQ ID NO: 33 - protein, 11 aa, Mus musculus
RLRPFWYFDVW

SEQ ID NO: 34 - protein, 16 aa, Mus musculus
RSSQSIVQSNGNTYLE

SEQ ID NO: 35 - protein, 7 aa, Mus musculus
KVSNRFS

SEQ ID NO: 36 - protein, 9 aa, Mus musculus
FQGSHVPRT

SEQ ID NO: 37 - protein, 30 aa, Mus musculus
EVKLLESGGGLVQPGGSLKLSCAASGFAFS

SEQ ID NO: 38 - protein, 14 aa, Mus musculus
WVRQAPGKGLEWIG

SEQ ID NO: 39 - protein, 32 aa, Mus musculus
KFIISRDNAKNTLSLQMNKVRSEDTALYYCAR

SEQ ID NO: 40 - protein, 10 aa, Mus musculus
GAGTTVTVSS

SEQ ID NO: 41 - protein, 23 aa, Mus musculus
DVLMTQTPLSLPVSLGDQASISC

SEQ ID NO: 42 - protein, 15 aa, Mus musculus
WYLQKPGQSPKLLIY

SEQ ID NO: 43 - protein, 32 aa, Mus musculus
GVPDRFSGSGSGTDFTLKISRVEAEDLGVYYC

SEQ ID NO: 44 - protein, 10 aa, Mus musculus
FGGGTKLEIK

SEQ ID NO: 45 - protein, 15 aa, Homo sapiens
NTTNLEFEICGALVE

SEQ ID NO: 46 - protein, 9 aa, Homo sapiens
LTCAFEDPD

SEQ ID NO: 47 - protein, 11 aa, Homo sapiens
PDHYFKGFWSE

SEQ ID NO: 48 - protein, 9 aa, Homo sapiens
LTCAFEDPD

SEQ ID NO: 49 - protein, 15 aa, Homo sapiens
NTTNLEFEICGALVE

SEQ ID NO: 50 - protein, 12 aa, Homo sapiens
PDHYFKGFWSEE

SEQ ID NO: 51 - protein, 119 aa, Mus musculus
DVQLQESGPGLVKPSQSLSLTCTVTGYSITTDYAWNWIRQFPGNKLEWMGYIFYSGSTTYTPSLKSRISITRDTSKNQFFLQLNSVTTEDTATYYCARGGYDVNYFDYWGQGTTLTVSS

SEQ ID NO: 52 - protein, 107 aa, Mus musculus
DIQMTQSPASQSASLGESVTITCLASQTIGAWLAWYQQKPGKSPQLLIYAATRLADGVPSRFSGSGSGTKFSFKISSLQAEDFVSYYCQQFFSTPWTFGGGTKLEIK

SEQ ID NO: 53 - protein, 6 aa, Mus musculus
TDYAWN

SEQ ID NO: 54 - protein, 16 aa, Mus musculus
YIFYSGSTTYTPSLKS

SEQ ID NO: 55 - protein, 8 aa, Mus musculus
GGYDVNYF

SEQ ID NO: 56 - protein, 11 aa, Mus musculus
LASQTIGAWLA

SEQ ID NO: 57 - protein, 7 aa, Mus musculus
AATRLAD

SEQ ID NO: 58 - protein, 9 aa, Mus musculus
QQFFSTPWT

SEQ ID NO: 59 - protein, 30 aa, Mus musculus
DVQLQESGPGLVKPSQSLSLTCTVTGYSIT

SEQ ID NO: 60 - protein, 14 aa, Mus musculus
WIRQFPGNKLEWMG

SEQ ID NO: 61 - protein, 32 aa, Mus musculus
RISITRDTSKNQFFLQLNSVTTEDTATYYCAR

SEQ ID NO: 62 - protein, 13 aa, Mus musculus
DYWGQGTTLTVSS

SEQ ID NO: 63 - protein, 23 aa, Mus musculus
DIQMTQSPASQSASLGESVTITC

SEQ ID NO: 64 - protein, 15 aa, Mus musculus
WYQQKPGKSPQLLIY

SEQ ID NO: 65 - protein, 32 aa, Mus musculus
GVPSRFSGSGSGTKFSFKISSLQAEDFVSYYC

SEQ ID NO: 66 - protein, 10 aa, Mus musculus
FGGGTKLEIK

SEQ ID NO: 67 - protein, 12 aa, Homo sapiens
TTNLEFEICGAL

SEQ ID NO: 68 - protein, 19 aa, Homo sapiens
IYFIETKKFLLIGKSNICV

SEQ ID NO: 69 - protein, 12 aa, Homo sapiens
TSHLQKKYVKVL

SEQ ID NO: 70 - protein, 11 aa, Homo sapiens
YFKGFWSEWSP

SEQ ID NO: 71 - protein, 119 aa, Mus musculus
EVQLQQSGPELLKPGASMKISCKASGYSFTGYTMNWVKQSHGKNLEWIGLINPYNGVTSYNQKFKGKATLTVAKSSSTAYMELLSLTSEDSAVYYCARGDGNYWYFDVWGAGTTVTVSS

SEQ ID NO: 72 - protein, 105 aa, Mus musculus
EIVLTQSPAITAASLGQKVTITCSASSSVTYMHWYQQKSGTSPKPWIYEISKLASGVPVRFSGSGSGTSYSLTISSMEAEDAAIYYCQEWNYPYTFGGGTKLEIK

SEQ ID NO: 73 - protein, 5 aa, Mus musculus
GYTMN

SEQ ID NO: 74 - protein, 16 aa, Mus musculus
LINPYNGVTSYNQKFK

SEQ ID NO: 75 - protein, 8 aa, Mus musculus
GDGNYWYF

SEQ ID NO: 76 - protein, 11 aa, Mus musculus
SASSSVTYMHW

SEQ ID NO: 77 - protein, 7 aa, Mus musculus
EISKLAS

SEQ ID NO: 78 - protein, 9 aa, Mus musculus
QEWNYPYTF

SEQ ID NO: 79 - protein, 30 aa, Mus musculus
EVQLQQSGPELLKPGASMKISCKASGYSFT

SEQ ID NO: 80 - protein, 14 aa, Mus musculus
WVKQSHGKNLEWIG

SEQ ID NO: 81 - protein, 33 aa, Mus musculus
GKATLTVAKSSSTAYMELLSLTSEDSAVYYCAR

SEQ ID NO: 82 - protein, 13 aa, Mus musculus
DVWGAGTTVTVSS

SEQ ID NO: 83 - protein, 23 aa, Mus musculus
EIVLTQSPAITAASLGQKVTITC

SEQ ID NO: 84 - protein, 14 aa, Mus musculus
YQQKSGTSPKPWIY

SEQ ID NO: 85 - protein, 32 aa, Mus musculus
GVPVRFSGSGSGTSYSLTISSMEAEDAAIYYC

SEQ ID NO: 86 - protein, 9 aa, Mus musculus
GGGTKLEIK

SEQ ID NO: 87 - protein, 21 aa, Homo sapiens
EVKCLNFRKLQEIYFIETKKF

SEQ ID NO: 88 - protein, 7 aa, Homo sapiens
FNTSHLQ

SEQ ID NO: 89 - protein, 14 aa, Homo sapiens
SIPDHYFKGFWSEW

SEQ ID NO: 90 - protein, 119 aa, Rattus
EVQLQQSGPELVKPGASMKISCKASGYSFTGYTMNWVKQSHGKNLEWIGLINPYSGITSYNQNFKGKATLTVDKSSSTAYMELLNLTSEDSAVYYCARGDGNYWYFDVWGAGTTVTVSS

SEQ ID NO: 91 - protein, 105 aa, Rattus
EIILTQSPAITAASLGQKVTITCSASSSVSYMHWYQQKSGTSPKPWIYEISKLASGVPARFSGSGSGTSYSLTISSMEAEDAAIYYCQYWNYPYTFGGGTKLEIK

SEQ ID NO: 92 - protein, 5 aa, Rattus
GYTMN

SEQ ID NO: 93 - protein, 16 aa, Rattus
LINPYSGITSYNQNFK

SEQ ID NO: 94 - protein, 8 aa, Rattus
GDGNYWYF

SEQ ID NO: 95 - protein, 11 aa, Rattus
SASSSVSYMHW

SEQ ID NO: 96 - protein, 7 aa, Rattus
EISKLAS

SEQ ID NO: 97 - protein, 9 aa, Rattus
QYWNYPYTF

SEQ ID NO: 98 - protein, 30 aa, Rattus
EVQLQQSGPELVKPGASMKISCKASGYSFT

SEQ ID NO: 99 - protein, 14 aa, Rattus
WVKQSHGKNLEWIG

SEQ ID NO: 100 - protein, 33 aa, Rattus
GKATLTVDKSSSTAYMELLNLTSEDSAVYYCAR

SEQ ID NO: 101 - protein, 13 aa, Rattus
DVWGAGTTVTVSS

SEQ ID NO: 102 - protein, 23 aa, Rattus
EIILTQSPAITAASLGQKVTITC

SEQ ID NO: 103 - protein, 14 aa, Rattus
YQQKSGTSPKPWIY

SEQ ID NO: 104 - protein, 32 aa, Rattus
GVPARFSGSGSGTSYSLTISSMEAEDAAIYYC

SEQ ID NO: 105 - protein, 9 aa, Rattus
GGGTKLEIK

SEQ ID NO: 106 - protein, 15 aa, Homo sapiens
NTTNLEFEICGALVE

SEQ ID NO: 107 - protein, 15 aa, Homo sapiens
VKCLNFRKLQEIYFI

SEQ ID NO: 108 - protein, 15 aa, Homo sapiens
ETKKFLLIGKSNICV

SEQ ID NO: 109 - protein, 15 aa, Homo sapiens
LQKKYVKVLMHDVAY

SEQ ID NO: 110 - protein, 15 aa, Homo sapiens
VLMHDVAYRQEKDEN

SEQ ID NO: 111 - protein, 40 aa, Homo sapiens
NTTNLEFEICGALVEVKCLNFRKLQEIYFIETKKFLLIGK

SEQ ID NO: 112 - protein, 13 aa, Homo sapiens
SHLQKKYVKVLMH

SEQ ID NO: 113 - protein, 4 aa, Homo sapiens
HYFK

SEQ ID NO: 114 - protein, 40 aa, Homo sapiens; identical to SEQ ID NO: 111

SEQ ID NO: 115 - protein, 13 aa, Homo sapiens
SHLQKKYVKVLMH

SEQ ID NO: 116 - protein, 4 aa, Homo sapiens
HYFK

SEQ ID NO: 117 - protein, 23 aa, Homo sapiens
SCYSQLEVNGSQHSLTCAFEDPD

SEQ ID NO: 118 - protein, 16 aa, Homo sapiens
NTTNLEFEICGALVEV

SEQ ID NO: 119 - protein, 22 aa, Homo sapiens
NFRKLQEIYFIETKKFLLIGKS

SEQ ID NO: 120 - protein, 22 aa, Homo sapiens
VTFNTSHLQKKYVKVLMHDVAY

SEQ ID NO: 121 - protein, 18 aa, Homo sapiens
EIKVRSIPDHYFKGFWSE

SEQ ID NO: 122 - protein, 3 aa, Homo sapiens
SQH

SEQ ID NO: 123 - protein, 3 aa, Homo sapiens
LVE

SEQ ID NO: 124 - protein, 7 aa, Homo sapiens
KKFLLIG

SEQ ID NO: 125 - protein, 2 aa, Homo sapiens
KY

SEQ ID NO: 126 - protein, 2 aa, Homo sapiens
YF

(19) United States
(12) Patent Application Publication
(10) Pub. No.: US 2011/0287000 A1
(43) Pub. Date: Nov. 24, 2011

(54) TREATMENT OF AUTOIMMUNE AND INFLAMMATORY DISEASE

(76) Inventors: Stewart Leung, Shanghai (CN); Lixin Li, Shanghai (CN); Xuebin Liu, Shanghai (CN); Hongtao Lu, Shanghai (CN); Ping Tsui, Shanghai (CN); Jingwu Zang, Shanghai (CN)

(21) Appl. No.: 13/057,893

(22) PCT Filed: Aug. 7, 2009
(86) PCT No.: PCT/US09/53136

§ 371 Date: Feb. 7, 2011

Related U.S. Application Data
(62) Division of application No. 61/087,294, filed on Aug. 8, 2008.
Application No. 61/087,294 is a division of application No. 61/169,801, filed on Apr. 16, 2009.
Application No. 61/169,801 is a division of application No. 61/218,627, filed on Jun. 19, 2009.

(51) Int. Cl. A61K 39/395; A61P 29/00; C40B 30/04; A61P 25/00; C07K 16/28; C07K 16/24

(52) U.S. Cl. 424/133.1; 424/158.1; 424/139.1; 530/387.3; 530/387.9; 530/389.2; 506/9

(57) ABSTRACT

The present invention provides novel methods of treatment of multiple sclerosis and other autoimmune diseases or inflammatory disorders, and antagonists, including isolated binding proteins for use in the novel methods. There is provided a method of treating multiple sclerosis comprising the neutralization of the biological activity of IL-7 by binding to CD127 or IL-7. The isolated binding proteins may also neutralize the biological activity of TSLP.

The present invention provides novel methods of treatment of multiple sclerosis and other autoimmune diseases, and novel isolated binding proteins for use in these methods. There is also provided a method of treating multiple sclerosis comprising the neutralization of the biological activity of IL-7 or IL-7R.

### BACKGROUND OF THE INVENTION

Multiple Sclerosis (MS) is a chronic inflammatory, demyelinating disease that affects the central nervous system. In MS, it is believed that infiltrating inflammatory immune cells are involved in the destruction of oligodendrocytes, which are the cells responsible for creating and maintaining a fatty layer, known as the myelin sheath. MS results in the thinning or complete loss of myelin. When the myelin is lost, the neurons can no longer effectively conduct their electrical signals leading to numerous neurologic dysfunctions. Individuals with MS produce autoreactive T cells that participate in the formation of inflammatory lesions along the myelin sheath of nerve fibres. The cerebrospinal fluid of patients with active MS contains activated T cells, which infiltrate the brain tissue and cause characteristic inflammatory lesions, destroying the myelin. While the multiple sclerosis symptoms and course of illness can vary from person to person, there are three forms of the disease—relapsing-remitting MS, secondary progressive MS, and primary progressive MS.

In the early stages of MS, inflammatory attacks occur over short intervals of acutely heightened disease activity. These episodes are followed by periods of recovery and remission. During the remission period, the local swelling in the nervous system lesion resolves, the immune cells become less active or inactive, and the myelin-producing cells remyelinate the axons. Nerve signalling improves, and the disability caused by the inflammation becomes less severe or goes away entirely. This phase of the disease is called relapsing-remitting MS (RRMS). The lesions do not all heal completely, though. Some remain as “chronic” lesions, which usually have a demyelinated core region which lacks immune cells. Over time, the cells in the centre of such lesions mostly die, although inflammation often continues at their edges. The brain can adapt well to the loss of some neurons, and permanent disability may not occur for many years. However, more than 50% of patients with MS eventually enter a stage of progressive deterioration, called secondary progressive MS (SPMS). In this stage, the disease no longer responds well to disease-modifying drugs, and patients' disabilities steadily worsen. The destruction of neurons from early in the natural course of MS suggests that the progressive disabilities of SPMS might be the result of an accumulated neuronal loss that eventually overwhelms the brain's compensatory abilities. Primary progressive MS is a type of multiple sclerosis where there are no relapses, but over a period of years, there is gradual loss of physical and cognitive functions.

The goal of treatment in patients with relapsing-remitting multiple sclerosis is to reduce the frequency and severity of relapses (and thereby prevent exacerbations) as well as to prevent or postpone the onset of the progressive phase of the disease. To achieve this goal, in the past especially, immunomodulatory or immunosuppressive drugs have been used, but they have never found widespread acceptance owing to limited efficacy and considerable toxicity. For example, large randomized controlled trials have been performed successfully with interferon beta-1a, interferon beta-1b, and glatiramer acetate.

Both altered autoimmune T cell responses and dysfunction of the regulatory network of the immune system play an important role in human autoimmune pathologies, such as MS and rheumatoid arthritis (Kuchroo et al., (2002) Annu. Rev. Immunol. 20:101-123; Sospedra and Martin (2005) Annu. Rev. Immunol. 23: 683-747; Toh and Miossec (2007) Curr. Opin. Rheumatol. 19:284-288).

Although the aetiology and pathogenesis of MS remain unknown, it is generally considered an autoimmune pathology in which autoreactive T cells of pathogenic potential, such as T_{H}1 and T_{H}17 cells, are thought to play an important role. There is evidence that these effector T cells are activated in vivo during the disease process and are attributable to the central nervous system (CNS) inflammation. There is also evidence that these T cells mediate destruction of myelin-expressing cells in lesions of EAE and MS during the active phase of the disease. On the other hand, regulatory T cells (T_{reg}) that normally keep pathogenic T_{H}1 and T_{H}17 cells in check are deficient in patients with MS, further tilting the immune system toward an pro-inflammatory state.

Three separate groups recently reported the results of genome wide single nucleotide polymorphisms (SNPs) scanning in a total of 17,947 donors with or without MS. After scanning 334,923 SNPs, they found a highly significant association (overall P=2.9×10−7) of a nonsynonymous coding SNP in the human IL-7 receptor alpha chain (IL-7Rα) with MS susceptibility. The SNP corresponds to a change from T to C in exon 6 of CD127 (also known as IL-7Rα). This change enhances the chance of exon 6 skipping during RNA splicing, resulting in a soluble form of CD127. Furthermore, expressions of CD127 and IL-7 RNAs in the cerebrospinal fluids (CSFs) of MS patients are significantly higher relative to CSFs of patients with other neurological disorders.

IL-7 and IL-7 receptor (IL-7R) are known to play an important role in T cell and B cell development and homeostasis mainly in a thymic environment. Indeed, thymic stromal cells, fetal thymus, and bone marrow are sites of IL-7 of production. The IL-7 receptor consists of two subunits, CD127 and a common chain (gamma chain or γc) which is shared by receptors of IL-2, IL-4, IL-9, IL-15, and IL-21.

CD127 is also known as IL-7 receptor alpha (IL-7Rα) and p90 IL-7R. Human CD127 (Swiss Prot accession number P16871) has a total of 459 amino acids (20 signal sequence). It comprises a 219 amino acid extra cellular region, a 25 amino acid transmembrane region and a 195 amino acid intracellular region. The numbering of residues within CD127, as used herein (e.g. for the description of antibody epitopes) is based on the full length protein, including signal sequence residues. CD127 may exist in four isoforms, the isoform H20 (Swissprot accession number P16871-1) has the following amino acid sequence (including signal sequence):

(SEQ ID NO: 1)
MTILGTTFGM VFSLLQVVSG ESGYAQNGDL EDAELDDYSF SCYSQLEVNG SQHSLTCAFE
DPDVNTTNLE FEICGALVEV KCLNFRKLQE IYFIETKKFL LIGKSNICVK VGEKSLTCKK
IDLTTIVKPE APFDLSVIYR EGANDFVVTF NTSHLQKKYV KVLMHDVAYR QEKDENKWTH
VNLSSTKLTL LQRKLQPAAM YEIKVRSIPD HYFKGFWSEW SPSYYFRTPE INNSSGEMDP
ILLTISILSF FSVALLVILA CVLWKKRIKP IVWPSLPDHK KTLEHLCKKP RKNLNVSFNP
ESFLDCQIHR VDDIQARDEV EGFLQDTFPQ QLEESEKQRL GGDVQSPNCP SEDVVVTPES
FGRDSSLTCL AGNVSACDAP ILSSSRSLDC RESGKNGPHV YQDLLLSLGT TNSTLPPPFS
LQSGILTLNP VAQGQPILTS LGSNQEEAYV TMSSFYQNQ

CD127 is also found in the receptor of thymic stromal derived lymphopoietin (TSLP). The TSLP receptor is a heterodimer of CD127 and cytokine receptor-like factor 2 (CRLF2).

Binding of IL-7 to the IL-7R activates multiple signaling pathways including the activation of JAK kinases 1 and 3 leading to the phosphorylation and activation of Stat5. This pathway is crucial to the survival of thymic developing T cell precursors because Stat5 activation is required in the induction of the anti-apoptotic protein Bcl-2 and the prevention of the pro-apoptotic protein Bax entry into the mitochondrion. Another IL-7R mediated pathway is the activation of PI3 kinase, resulting in the phosphorylation of the pro-apoptotic protein Bad and its cytoplasm retention. CD127 is expressed in peripheral resting and memory T cells. The mechanism of IL-7 regulation of T cell survival and homeostasis and the source of IL-7 in the periphery are not completely understood. Furthermore, its potential role in the differentiation and function of pathogenic T cells in autoimmune disease is poorly studied and largely unknown. There are few reports suggesting that IL-7 may contribute to the pathogenesis of autoimmune diseases.

CD127 has been described in W09015870 and antagonists of IL-7 and CD127 in the treatment of multiple sclerosis have been described in WO2006052660 and US20060198822. Antagonists of TSLP have been described in, for example, U.S. Pat. No. 7,304,144 and WO2007096149.

### SUMMARY OF THE INVENTION

The present inventors have shown that IL-7/CD127 antagonism is efficacious in amelioration of Experimental Autoimmune Encephalomyelitis (EAE). The treatment resulted in marked reduction of T_{H}17 and, to a lesser degree, T_{H}1 cells in both spleen and spinal cord of treated mice, which was accompanied by an increased level of Foxp3+ T_{reg}. The inventors have also shown that IL-7 is critically required for the expansion and survival of T_{H}17 cells, but that its requirement during differentiation of precursor T cells into a T_{H}17 cell population is minimal.

Restoring the balance of the functional ratio of autoreactive inflammatory T_{H}17 and T_{H}1 cells and T_{reg} with an antagonist of CD127 or IL-7 provides great potential as a therapy for multiple sclerosis and other autoimmune diseases.

The selective susceptibility of T_{H}17 and T_{H}1 cells was attributable to high expression of CD127 in activated pathogenic T cells and their requirement for IL-7 for expansion and survival. Blockade of CD127 led to altered signalling events characterized by down-regulation of phosphorylated JAK-1 and STAT-5 and BCL-2 and the increased activity of BAX, rendering CD127+ T_{H}17 and T_{H}1 cells susceptible to apoptosis. In contrast, Foxp3+ T_{reg} (inducible T_{reg}) were resistant to CD127 antagonism as they did not express, or expressed lower levels of, CD127. Signalling events, including apoptotic pathways, downstream to IL-7/IL-7R interaction were not affected in Foxp3₊ T_{reg} by a neutralizing anti-CD127 antibody. Furthermore, similar effects of CD127 antagonism were seen in human T_{H}17 and T_{H}1 expansion and survival, which spared T_{reg}. These findings provide new evidence supporting the role of IL-7 in pathogenic T cell differentiation and maintenance and have important therapeutic implications in MS and other human autoimmune diseases.

Therefore, in a first aspect of the invention, there is provided a method for the treatment of an autoimmune disease or an inflammatory disorder in a human subject, comprising administering to the subject an antagonist of at least one of: IL-7 receptor mediated T_{H}17 expansion, and IL-7 receptor mediated T_{H}17 survival.

IL-7 receptor mediated T_{H}17 expansion and/or survival can be observed at a cellular level by an increase or maintenance of T_{H}17 cell count, or by an increase in the ratio of T_{H}17 cell numbers compared to the numbers of other CD4₊ T cells, or more specifically by an increase in the T_{H}17:T_{H}1 ratio, the T_{H}17:T_{reg} ratio, the (T_{H}17 plus T_{H}1):T_{reg} ratio, and/or the T_{H}17:(T_{H}1 plus T_{reg}) ratio.

At a molecular level, T_{H}17 expansion and/or survival can be observed by an increase in IL-17 production by a population of CD4+ T cells (or by a population of T_{H}17 cells). In an embodiment, therefore, the antagonist of IL-7 receptor mediated T_{H}17 expansion and/or IL-7 receptor mediated T_{H}17 survival reduces IL-17 production by a population of CD4+ T cells. IL-7 receptor mediated T_{H}17 expansion and survival can also be observed by an increase in IFN-γ production by a population of CD4+ T cells (or by a population of T_{H}17 cells). Thus, in an embodiment, the antagonist of the present invention inhibits IFN-γ production by a population of CD4+ T cells. At a molecular level, the antagonist of IL-7 receptor mediated T_{H}17 expansion and/or survival may inhibit IL-7 receptor mediated STAT-5 phosphorylation.

Thus, in another aspect, the invention provides a method for the treatment of an autoimmune disease or inflammatory disorder, comprising administering to a patient a antagonist of IL-7 or CD127 in an amount sufficient to reduce the T_{H}17 cell count in the patient.

In another aspect, the invention provides a method for the treatment of an autoimmune disease in a human subject, comprising administering to the subject an antagonist of IL-7 receptor mediated STAT-5 phosphorylation.

In another aspect, the present invention provides a method for treating multiple sclerosis in a patient comprising administering an antagonist of IL-7 or CD127 to said patient, wherein the patient is suffering from relapsing remitting multiple sclerosis.

In another aspect, the invention provides a method of treating an autoimmune or inflammatory disease in a human subject, comprising administering to the subject an antagonist of IL-7 or IL-7R in an amount effective to reduce the ratio of T_{H}17 cells relative to T_{H}1 cells.

In another aspect, the invention provides a method of treating an autoimmune or inflammatory disease in a human subject, comprising administering to the subject an antagonist of IL-7 or IL-7R in an amount effective to reduce the ratio of T_{H} cells relative to (Foxp3+) T_{reg} cells.

In an embodiment of the above methods, the antagonist is selected from the group consisting of (a) a binding protein which specifically binds to CD127 (SEQ ID NO:1); (b) a binding protein which specifically binds to IL-7, (c) a soluble CD127 polypeptide; and (d) a combination of two or more of said antagonists.

In an embodiment, the binding protein which specifically binds CD127 or IL-7 is an isolated human, humanized or chimeric antibody. In an embodiment, the binding protein which specifically binds to CD127 (an anti-CD127 binding protein) is an antibody, or an antigen-binding fragment thereof. In some embodiments, the anti-CD127 binding protein inhibits the binding of IL-7 to the IL-7R receptor complex.

Certain anti-CD127 antibodies useful in the methods of the present invention are described herein, and include 9B7, 6C5, 6A3, R34.34, GR34 and 1A11, humanised or chimeric versions thereof, analogs thereof, and antigen-binding fragments thereof.

In an embodiment, the binding protein which specifically binds to IL-7 (an anti-IL-7 binding protein) is an antibody, or an antigen-binding fragment thereof.

In another aspect, the invention provides a chimeric, humanised or fully human antibody or an antigen-binding fragment thereof which binds to CD127 and which is capable of inhibition of IL-7 mediated T_{H}17 expansion.

The present inventors have determined that anti-CD127 binding proteins are not uniformly effective at functionally neutralising the IL-7 pathway or IL-7R mediated signalling. On the contrary, there are certain regions of the human CD127 polypeptide which appear to play an important role in the signalling pathway, to the extent that an antibody which is capable of binding to one or more of these regions of human CD127 is particularly effective in neutralising the IL-7 pathway or IL-7R mediated signalling. These regions are defined by amino acid residues:

(i)

| | |
|---|---|
| 41 SCYSQLEVNGSQHSLTCAFEDPD 63, | (SEQ ID NO: 117) |

(ii)

| | |
|---|---|
| 65 NTTNLEFEICGALVEV 80, | (SEQ ID NO: 118) |

(iii)

| | |
|---|---|
| 84 NFRKLQEIYFIETKKFLLIGKS 105, | (SEQ ID NO: 119) |

(iv)

| | |
|---|---|
| 148 VTFNTSHLQKKYVKVLMHDVAY 169, | (SEQ ID NO: 120) |

and
(v)

| | |
|---|---|
| 202 EIKVRSIPDHYFKGFWSE 219 | (SEQ ID NO: 121) |

of SEQ ID NO: 1.

It is postulated that these regions contain amino acids which play a role in the interaction between the ligand IL-7 and the CD127 receptor. The following amino acids are believed to be of particular significance in the IL-7/CD127 interaction: amino acids

| | |
|---|---|
| (a) 51 SQH 53, | (SEQ ID NO: 122) |
| (b) 77 LVE 79, | (SEQ ID NO: 123) |
| (c) 97 KKFLLIG 103, | (SEQ ID NO: 124) |
| (d) 158 KY 159, | (SEQ ID NO: 125) |
| and | |
| (e) 212 YF 213. | (SEQ ID NO: 126) |

Binding more than one of these regions may be of significance in the inhibition of IL-7R function.

In an embodiment, the antigen-binding proteins are capable of binding to at least one amino acid within, or an amino acid flanking or structurally neighbouring, at least one or a plurality of regions (i) to (iv) as defined above. In another embodiment, the antigen-binding proteins are capable of binding to at least one amino acid within, or an amino acid, at least one of the regions (a) to (e), as defined above.

In an embodiment, the invention provides antigen-binding proteins which are capable of binding to at least one amino acid within a region defined by amino acid residues 202 to 219 of SEQ ID NO:1. The antigen-binding protein according to this embodiment may further be capable of binding to at least one amino acid within one, two, three or all four of the regions defined by amino acid residues (i) 41 to 63, (ii) 65 to 80, (iii) 84 to 105 and (iv) 148 to 169 of SEQ ID NO:1.

In an embodiment, the antigen-binding protein binds to at least one amino acid within a region defined by amino acids (v) 202 to 219 of SEQ ID NO:1 and at least one amino acid within a region defined by amino acids (iv) 148 to 169 of SEQ ID NO:1. The antigen-binding protein according to this embodiment may further be capable of binding to at least one amino acid within a region defined by amino acids (ii) 65 to 80 and/or (iii) 84 to 105 of SEQ ID NO:1. In a particular embodiment, the antigen-binding protein binds to at least one amino acid within each of peptides (ii) 65 to 80, (iii) 84 to 105, (iv) 148 to 169, and (v) 202 to 219 of SEQ ID NO:1.

In another embodiment, the invention provides antigen-binding proteins which are capable of binding to at least one amino acid within a region defined by amino acid residues (e) 212 to 213 of SEQ ID NO:1, or a flanking or structurally neighbouring amino acid. The antigen-binding protein according to this embodiment may further be capable of binding to at least one amino acid within, flanking or structurally neighbouring to, one, two, three or all four of the regions defined by amino acid residues (a) 51 to 53, (b) 77 to 79, (c) 97 to 103 and (d) 158 to 159 of SEQ ID NO:1.

In an embodiment, the binding protein binds to at least one amino acid within a region defined by amino acids (e) 212 to 213 of SEQ ID NO:1, or a flanking or structurally neighbouring amino acid, and at least one amino acid within, flanking, or structurally neighbouring to a region defined by amino acids (d) 158 to 159 of SEQ ID NO:1. The binding protein according to this embodiment may further be capable of binding to at least one amino acid within, flanking or structurally neighbouring to a region defined by amino acids (b) 77 to 79 and/or (c) 97 to 103 of SEQ ID NO:1. In a particular embodiment, the binding protein binds to at least one amino acid within each of peptides (b) 77 to 79, (c) 97 to 103, (d) 158 to 159, and (e) 212 to 213 of SEQ ID NO:1.

Antibodies according to these aspects of the invention include 6A3, 1A11, 6C5 and 9B7, antigen-binding fragments thereof and chimeric or humanised variants thereof. Additional antibodies of these aspects of the invention are chimeric or humanised variants of R3434 or GR34, or an antigen-binding fragment of R3434 or GR34.

In another aspect, the invention provides a human, humanised or chimeric antibody, or a fragment thereof, wherein the antibody or fragment binds to an epitope of human CD127 that contains at least one amino acid residue within the region beginning at residue number 80 and ending at residue number 190.

In an embodiment, the invention provides an antibody or fragment thereof which binds to an epitope of human CD127 (SEQ ID NO:1), wherein said epitope has an amino acid residues which are present in at least one of the regions of CD127 of SEQ ID NOs:20-28, 45-50,67-70, 87-89, and 106-116. This binding may be measured by, inter alia, peptide ELISA, surface plasmon resonance (BIAcore) or phage display.

In particular embodiments, the antibody or fragment thereof binds to an epitope of human CD127 (SEQ ID NO:1), wherein said epitope has amino acid residues which are present in: one, two, three or four of the regions of SEQ ID NOs:66-70; one, two or three of the regions of CD127 of SEQ ID NOs:87-89; or one, two or three of the regions of CD127 of SEQ ID NOs:114-116.

In an embodiment, the invention provides an antibody or fragment thereof which binds to an epitope of human CD127, wherein said epitope has an amino acid residue present in at least one of the following regions of CD127: 35-49 (SEQ ID NO:20), 84-105 (SEQ ID NO:21) 171-180 (SEQ ID NO:22), or an antibody or fragment which binds to an at least one of the following linear peptides: 35-49 (SEQ ID NO:20), 84-105 (SEQ ID NO:21) 171-180 (SEQ ID NO:22). This binding may be measured by, inter alia, peptide ELISA, surface plasmon resonance (BIAcore), or phage display. In an embodiment, the invention provides an antibody or fragment thereof which binds to an epitope of human CD127 (SEQ ID NO:1), the epitope having an amino acid residue present within, or the epitope being present within the following regions of CD127 (SEQ ID NO:1): 80-94 (SEQ ID NO:23), 95-109 (SEQ ID NO:24), 170-184 (SEQ ID NO:25). In an embodiment, the invention provides an antibody or fragment thereof which binds to an epitope of human CD127 (SEQ ID NO:1), the epitope having an amino acid residue present within, or the epitope being present within the following regions of CD127 (SEQ ID NO:1): 35-49 (SEQ ID NO:26), 84-105 (SEQ ID NO:27), 139-184 (SEQ ID NO:28).

In another aspect of the invention, there is provided an antibody or fragment thereof which binds to a C-terminal biotinylated CD127 peptide that comprises residues 35-49, 84-105, 171-180 of CD127 as determined by surface plasmon resonance, said peptide being bound to a streptavidin sensor chip.

In another embodiment, the antibody or fragment thereof additionally requires at least one flanking residue or structurally neighbouring residue to said at least one residue in the 35-49, 84-105 or 171-180 regions of CD127 for binding.

The person skilled in the art can readily identify such antibodies or fragments thereof using, for example, alanine replacement scanning in ELISA assays. In this respect, whether or not the antibody requires a residue in the abovedefined regions of CD127, or a flanking or structurally neighbouring residue, for binding can be determined by independently substituting said residue of CD127 with alanine and comparing the binding affinity of the antibody to the alanine substituted CD127 peptide with the binding affinity of the antibody to the wild type CD127. Whether or not a residue in the abovedefined regions of CD127 is required is defined by a reduction in binding affinity of the antibody to the alanine substituted CD127 compared with the wild-type CD127, wherein said reduction is more than 1, 2, 3, 4 or 5 fold as determined by Biacore or ELISA affinity measurements.

Further, a structurally neighbouring residue in this context is a residue that is in close proximity in three-dimensional space to the residue in question and which is bound by the antibody. The person skilled in the art appreciates that antigen epitopes may be either liner or non-liner peptide sequences. In the latter, non-linear case, although the residues are from different regions of the peptide chain, they may be in close proximity in the three dimensional structure of the antigen. Such structurally neighbouring residues can be determined through computer modelling programs or via three-dimensional structures obtained through methods known in the art, such as X-ray crystallography.

Another aspect of the present invention relates to therapeutic antibodies and antigen-binding fragments thereof which are specific for CD127, and which are useful in the treatment of autoimmune and/or inflammatory disorders. The antibodies and antigen-binding fragments may inhibit T_{H}17 expansion and survival and/or inhibit pSTAT-5, in an assay that as that herein defined. These antibodies and antigen-binding fragments may represent the antagonist useful in the methods of the invention.

More particularly, in one aspect, there is provided an antibody or antigen-binding fragment and/or derivative thereof which binds to CD127 and which comprises at least a third heavy chain CDR (CDRH3) selected from the group consisting of: 9B7-CDRH3 (SEQ ID NO:6); 6C5-CDRH3 (SEQ ID NO:33), 6A3-CDRH3 (SEQ ID NO:55) or 1A11-CDRH3 (SEQ ID NO:75).

In an embodiment, the antibody or antigen-binding fragment and/or derivative thereof comprises CDRH3 of: antibody 9B7 (SEQ ID NO:6) and one, two, three, four or all five additional CDRs of 9B7 (SEQ ID NOs:4,5,7,8,9); antibody 6C5 (SEQ ID NO:33) and one, two, three, four or all five additional CDRs of 6C5 (SEQ ID NOs: 31,32,34,35,36); antibody 6A3 (SEQ ID NO:55) and one, two, three, four or all five additional CDRs of 6A3 (SEQ ID NOs: 53,54,56,57,58); or antibody 1A11 (SEQ ID NO:75) and one, two, three, four or all five additional CDRs of 1A11 (SEQ ID NOs:73,74,76,77,78).

In another aspect there is provided a therapeutic antibody which is an antibody or an antigen binding fragment and/or derivative thereof which binds to CD127 and which comprises the following CDRs, or analogs thereof:

| | | | |
|---|---|---|---|
| A: | CDRH1: | RYNVH; | (SEQ ID NO: 4) |
| | CDRH2: | MIWDGGSTDYNSALKS; | (SEQ ID NQ: 5) |
| | CDRH3: | NRYESG; | (SEQ ID NO: 6) |
| | CDRL1: | KSSQSLLNSGNRKNYLT; | (SEQ ID NO: 7) |
| | CDRL2: | WASTRES; | (SEQ ID NO: 8) |
| | | and | |
| | CDRL3: | QNDYTYPFTFGS. | (SEQ ID NO: 9) |
| B: | CRDH1: | AYWMS | (SEQ ID NO: 31) |
| | CDRH2: | EINPDSSTINCTPSLKD | (SEQ ID NO: 32) |
| | CDRH3: | RLRPFWYFDVW | (SEQ ID NO: 33) |
| | CDRL1: | RSSQSIVQSNGNTYLE | (SEQ ID NO: 34) |
| | CDRL2: | KVSNRFS | (SEQ ID NO: 35) |
| | CDRL3: | FQGSHVPRT | (SEQ ID NO: 36) |
| C: | CRDH1: | TDYAWN | (SEQ ID NO: 53) |
| | CDRH2: | YIFYSGSTTYTPSLKS | (SEQ ID NO: 54) |
| | CDRH3: | GGYDVNYF | (SEQ ID NO: 55) |
| | CDRL1: | LASQTIGAWLA | (SEQ ID NO: 56) |
| | CDRL2: | AATRLAD | (SEQ ID NO: 57) |
| | CDRL3: | QQFFSTPWT | (SEQ ID NO: 58) |
| D: | CDRH1: | GYTMN | (SEQ ID NO: 73) |
| | CDRH2: | LINPYNGVTSYNQKFK | (SEQ ID NO: 74) |
| | CDRH3: | GDGNYWYF | (SEQ ID NO: 75) |
| | CDRL1: | SASSSVTYMHW | (SEQ ID NO: 76) |
| | CDRL2: | EISKLAS | (SEQ ID NQ: 77) |
| | CDRL3: | QEWNYPYTF. | (SEQ ID NO: 78) |

In another aspect there is provided a therapeutic antibody which is a human, humanised or chimeric antibody or an antigen binding fragment and/or derivative thereof which binds to CD127 and which comprises the following CDRs, or analogs thereof:

| | |
|---|---|
| CDRH1: GYTMN | (SEQ ID NO: 92) |
| CDRH2: LINPYSGITSYNQNFK | (SEQ ID NO: 93) |
| CDRH3: GDGNYWYF | (SEQ ID NO: 94) |
| CDRL1: SASSSVSYMHW | (SEQ ID NO: 95) |
| CDRL2: EISKLAS | (SEQ ID NO: 96) |
| CDRL3: QYWNYPYTF. | (SEQ ID NO: 97) |

Throughout this specification, the terms “CDR”, “CDRL1”, “CDRL2”, “CDRL3”, “CDRH1”, “CDRH2”, “CDRH3” follow the Kabat numbering system, as set forth in Kabat et al; Sequences of proteins of Immunological Interest NIH, 1987. Therefore the following defines the CDRs according to the invention:

| CDR | Residues |
|---|---|
| CDRH1 | 31-35, 35(A), 35(B) |
| CDRH2 | 50-65 |
| CDRH3 | 95-97 |
| CDRL1 | 24-34 |
| CDRL2 | 50-56 |
| CDRL3 | 80-97 |

In another aspect, there is provided a monoclonal antibody comprising:
(i) the heavy chain variable region of SEQ ID NO:2 and/or the light chain variable region of SEQ ID NO:3;
(ii) the heavy chain variable region of SEQ ID NO:29 and/or the light chain variable region of SEQ ID NO:30;
(iii) the heavy chain variable region of SEQ ID NO:51 and/or the light chain variable region of SEQ ID NO:52; or
(iv) the heavy chain variable region of SEQ ID NO:71 and/or the light chain variable region of SEQ ID NO:72.

Also provided by the present invention are antibody variable domain sequences that have at least 90% identity, or at least 95% identity, or at least 98% identity, or at least 99% identity, over the whole length of the sequences of SEQ ID NOs: 2, 3, 29, 30, 51, 52, 71, and 72.

Also provided by the invention is a method of treatment of an autoimmune disease or inflammatory disorder comprising administering to a patient an anti-CD127 antibody, wherein the antibody comprises:
(i) the heavy chain variable region of SEQ ID NO:2 and/or the light chain variable region of SEQ ID NO:3;
(ii) the heavy chain variable region of SEQ ID NO:29 and/or the light chain variable region of SEQ ID NO:30;
(iii) the heavy chain variable region of SEQ ID NO:51 and/or the light chain variable region of SEQ ID NO:52;
(iv) the heavy chain variable region of SEQ ID NO:71 and/or the light chain variable region of SEQ ID NO:72; or
(v) the heavy chain variable region of SEQ ID NO:90 and/or the light chain variable region of SEQ ID NO:91,
   or a monoclonal antibody having a heavy and light chain variable regions that have at least 90% identity, or at least 95% identity, or at least 98% identity, or at least 99% identity, to these heavy and/or light chain variable regions.

In another aspect, the invention provides an antibody or an antigen-binding fragment thereof which binds to CD127 and which is capable of inhibition of IL-7 mediated T_{H}17 expansion, wherein the antibody is not R.34.34 (Dendritics Inc., #DDX0700).

In another aspect of the present invention, there is provided a method for identifying antibodies or antibody fragment suitable for use in the treatment of an autoimmune disease or an inflammatory disease, the method comprising the steps of: screening a plurality of independent antibody or antibody fragment populations to determine the ability of each antibody population to:
i. inhibit the binding of IL-7 to IL-7R,
ii. neutralise IL-7 induced STAT-5 phosphorylation, and/or
iii. inhibit the production of IL-17 by T_{H}17 cells, and selecting those antibody or antibody fragment populations which are able to inhibit the binding of IL-7 to IL-7R, inhibit IL-7 induced STAT-5 phosphorylation, and/or inhibit the production of IL-17 by T_{H}17 cells in vivo.

The ability of a composition or substance (a test agent) to act as an antagonist of IL-7 receptor mediated T_{H}17 expansion or IL-7 receptor mediated T_{H}17 survival, or to reduce T_{H}17 cell count, can be determined by routine methods. For example, naïve CD4+ cells can be stimulated to differentiate into T_{H}17 with appropriate conditions known to those of skill in the art (e.g. TGF-β1, IL-23, IL-6, anti-IFN-γ and anti-IL-4, or IL-1β, IL-6 and IL-23). A T_{H}17 population of cells can then be exposed to the test agent and IL-7, following which the T_{H}17 cell count can be determined. A decrease in T_{H}17 cells relative to a control would indicate that the test agent is capable of inhibiting T_{H}17 expansion or survival.

In another aspect of the invention, there is provided a method of manufacturing a medicament for the treatment of autoimmune or inflammatory disease, the method comprising formulating an anti-CD127 or anti-IL-7 antibody or antigen-binding fragment thereof and one or more excipients into a pharmaceutically acceptable formulation. This method may comprise the preliminary steps of identifying an antibody, as hereinbefore defined, and/or of recombinantly producing such an antibody.

In the definitions of the epitopes of CD127 that are bound by the binding proteins and antibodies of the present invention, the numbering system used refers to the full length sequence of CD127, which includes the signal sequence. In one embodiment the epitopes of human CD127 are found within the cited residues of SEQ ID NO:1.

In one embodiment, the binding proteins of the present invention binds to human CD127 with an affinity (KD) which is less than 20 nM, less than 15 nM, less than 10 nM, less than 5 nM, less than 1 nM or less than 0.5 nM, as measured by surface plasmon resonance (BIAcore).

In an embodiment, the binding protein competitively inhibits binding of 9B7, 6C5, 3A6, 1A11 or R34.34 (Dendritics Inc. #DDX0700), or an antigen-binding fragment thereof to human CD127.

Competitive inhibition can be determined by those skilled in the art, for example, in a competition ELISA assay, by BIAcore or Scatchard analysis.

In one aspect of the present invention, there are provided isolated binding proteins which compete with:
i. antibody R34.34 (Dendritics Inc., #DDX0700);
ii. an antibody having a variable heavy chain region as set out in SEQ ID NO:2 and a variable light chain region as set out in SEQ ID NO:3;
iii. an antibody having a variable heavy chain region as set out in SEQ ID NO:29 and a variable light chain region as set out in SEQ ID NO:30;
iv. an antibody having a variable heavy chain region as set out in SEQ ID NO:51 and a variable light chain region as set out in SEQ ID NO:52;
v. an antibody having a variable heavy chain region as set out in SEQ ID NO:71 and a variable light chain region as set out in SEQ ID NO:72; or
vi. an antibody having a variable heavy chain region as set out in SEQ ID NO:90 and a variable light chain region as set out in SEQ ID NO:91,
   for binding to CD127, wherein the antibody is not R.34.34 (Dendritics Inc., #DDX0700).

In a particular embodiment, the isolated binding protein of the present invention is an antibody or an antigen-binding fragment thereof which competes with:
i. antibody R34.34 (Dendritics Inc., #DDX0700);
ii. an antibody having a variable heavy chain region as set out in SEQ ID NO:51 and a variable light chain region as set out in SEQ ID NO:52;
iii. an antibody having a variable heavy chain region as set out in SEQ ID NO:71 and a variable light chain region as set out in SEQ ID NO:72; or
iv. an antibody having a variable heavy chain region as set out in SEQ ID NO:90 and a variable light chain region as set out in SEQ ID NO:91,
   for binding to CD127, wherein the antibody is not R.34.34 (Dendritics Inc., #DDX0700).

Also provided by the present invention are binding proteins for use in the treatment of multiple sclerosis, wherein the binding proteins compete for binding to human CD127 (SEQ ID NO:1) with:
i. antibody R34.34 (Dendritics Inc., #DDX0700);
ii. an antibody having a variable heavy chain region as set out in SEQ ID NO:2 and a variable light chain region as set out in SEQ ID NO:3;
iii. an antibody having a variable heavy chain region as set out in SEQ ID NO:29 and a variable light chain region as set out in SEQ ID NO:30;
iv. an antibody having a variable heavy chain region as set out in SEQ ID NO:51 and a variable light chain region as set out in SEQ ID NO:52;
v. an antibody having a variable heavy chain region as set out in SEQ ID NO:71 and a variable light chain region as set out in SEQ ID NO:72; or
vi. an antibody having a variable heavy chain region as set out in SEQ ID NO:90 and a variable light chain region as set out in SEQ ID NO:91,
   for binding to CD127.

The person skilled in the art appreciates that in order for an antibody or fragment (antibody or fragment A) to compete with antibody R34.34, GR34, 6A3, 1A11, 6C5 or 9B7 (antibody B) for a specific binding site (of human CD127), antibody A must be present in a sufficient amount to have an effect in said assay. For example, antibody A and antibody B may be present in equimolar amounts. If antibody A is a competing antibody, the presence of antibody A may reduce the binding of antibody B to human CD127 in an ELISA assay by more than 10%, 20%, 30%, 40% or 50%. A competing antibody (antibody A) may reduce the binding of antibody B to plate-bound human CD127, whereas a non-anti-CD127-specific control does not. In such ELISA assays human CD127 may be bound to an immunoassay plate. In another assay system, surface plasmon resonance may be used to determine competition between antibodies.

Isolated binding proteins which are capable of competition for binding to CD127 with antibody R34.34 or the antibodies of the invention, an isolated binding protein having a V_{H} of SEQ ID NO:2 and V_{L} of SEQ ID NO:3, an isolated binding protein having a V_{H} of SEQ ID NO:76 and a V_{L} of SEQ ID NO:77, or an isolated binding protein having a V_{H} of SEQ ID NO:193 and a V_{L} of SEQ ID NO:194 may be used in the treatment of MS and other autoimmune diseases.

The binding proteins of the present invention may comprise the CDRs of R34.34, GR34, 9B7, 6A3, 1A11 or 6C5, or they may comprise analogs thereof.

Also provided by the present invention are humanized antibodies, wherein the R34.34, GR34, 9B7, 6A3, 1A11 or 6C5 CDRs (or analogs thereof) are grafted into a heavy chain or light chain variable domain framework.

In another aspect of the present invention there is provided a polynucleotide sequence which encodes the binding proteins of the present invention. In particular, there is provided a polynucleotide sequence that encodes an antibody or fragment thereof which comprises one or all of the CDRs found in 9B7 (SEQ ID NOs:4-9), 6C5 (SEQ ID NOs:31-36), 6A5 (SEQ ID NOs:53-58), 1A11 (SEQ ID NOs:73-78) or GR34 (SEQ ID NOs:92-97). In a related aspect of the present invention there is provided a host cell transfected with the polynucleotides of the present invention.

The binding proteins, antibodies, antigen-binding fragments, their humanised, human or chimeric variants, and analogs, of the present invention may be used in a method of treatment of multiple sclerosis, the method comprising administering a safe and effective dose of the binding proteins of the present invention to a patient in need thereof. In this aspect of the present invention the binding protein may be an antibody which comprises one or all of the CDRs found in 9B7 (SEQ ID NOs:4-9), 6C5 (SEQ ID NOs:31-36), 6A5 (SEQ ID NOs:53-58), 1A11 (SEQ ID NOs:73-78) or GR34 (SEQ ID NOs:92-97).

Also provided in this aspect of the present invention is a method where the patient in need of the treatment is a relapsing/remitting MS (RRMS) patient who is about to, or is in, a relapse phase.

In another aspect, the invention provides a method of treating an autoimmune or inflammatory disease comprising administering to a subject in need thereof a therapeutically effective amount of an antagonist of IL-7 or IL-7R and an additional therapeutic agent.

The additional therapeutic agent may be selected from the group consisting of: immunomodulators such as interferon beta (IFNβ-1a or IFNβ-1b) and glatiramer acetate, immunosuppresants such as cyclophosphamide, methotrexate, azathioprine, cladribine, cyclosporine and mitoxantrone, other immune therapies such as intravenous immune globulin (IVIg), plasma replacement and sulphasalazine. The additional therapeutic may be administered as in a manner (dosage, timing, mechanism) as prescribed by a physician. In an embodiment, the additional therapeutic agent may be administered simultaneously or sequentially or separately from the antagonist of the present invention. In an embodiment, the additional therapeutic agent and the antagonist are administered such that their pharmacological effects on the patient overlap; in other words, they exert their biological effects on the patient at the same time.

In another embodiment of the invention, the IL7/IL7R antagonist is a soluble CD127 polypeptide. The soluble CD127 polypeptide may comprise a polypeptide which is 90% or more identical to a polypeptide selected from the extracellular domain of CD127 (SEQ ID NO:1), or a polypeptide comprised of amino acids 21 to 219 of SEQ ID NO:1. In certain embodiments, the soluble CD127 comprises a polypeptide is amino acids 21-219 of SEQ ID NO:1. In further embodiments, the soluble CD127 polypeptide may be fused to a non-CD127 moiety. The non-CD127 moiety may be a heterologous peptide fused to the soluble CD127 polypeptide. In an embodiment, the non-CD127 moiety is selected from the group consisting of serum albumin, a targeting protein, an immunoglobulin fragment, a reporter protein or a purification-facilitating protein. In a particular embodiment, the soluble CD127 polypeptide is fused to an Fc region of an immunoglobulin.

### BRIEF DESCRIPTION OF THE FIGURES

FIG. 1(A) shows inhibition of IL-7-mediated pSTAT5 by anti-mouse CD127 antibodies;

FIG. 1(B) shows inhibition of TSLP mediated pSTAT5 by anti-mouse CD127 antibodies;

FIG. 2 shows a CD127 ELISA binding curve for 9B7;

FIG. 3(A) shows that MAb 9B7 (solid line) is capable of recognizing CD127 expressed on the surface of CD127-transfected CHO cell line. An irrelevant, isotype control, antibody is shown as a dotted line;

FIG. 3(B) shows that antibody 9B7 (solid line) is not capable of recognizing CD127 in a mock transfected CHO cell line—an irrelevant, isotype control, antibody is shown as a dotted line;

FIG. 4 demonstrates an example of the inhibition of IL7-mediated pStat5 signalling by purified murine anti-CD127 mAb 9B7;

FIG. 5(A) shows that the MOG-EAE clinical score was ameliorated by rat anti-murine CD127 antibody SB/14;

FIG. 5(B) shows inhibition of MOG peptide-induced T-cell proliferation by SB/14;

FIG. 5(C) shows inhibition of cytokine production by anti-CD127 antibody by SB/14;

FIGS. 5(D) and 5(E) show the selective effect of anti-mCD127 antibody (SB/14) treatment on helper T cell subtypes;

FIG. 5(F) shows that the MOG-EAE clinical score was ameliorated by anti-mCD127 antibody (SB/14) treatment;

FIG. 6 shows CD127 expression in T_{reg}, T_{H}1 and T_{H}17 cells derived ex vivo from spleen or spinal cord of EAE mice;

FIG. 7(A) shows that the effect of IL-7 on the promotion of T_{H}17 differentiation was modest compared to that of IL-6;

FIG. 7(B) shows that the induction of STAT-3 phosphorylation is largely driven by IL-6 independently from IL-7;

FIG. 7(C) shows that the effect of IL-7 on RORα expression is also modest compared to that of IL-6;

FIG. 7(D) shows that the effect of anti-mCD127 antibody (SB/14) treatment was modest during disease onset in EAE;

FIG. 8(A) shows the percentage of T_{H}17 cells, γ-interferon secreting T_{H}1 cells, and T_{reg} cells in the CNS;

FIG. 8(B) shows the percentage of T_{H}17 cells, γ-interferon secreting T_{H}1 cells, and T_{reg} cells in splenocytes;

FIG. 8(C) shows the percentage of T_{H}17, T_{H}1 and T_{reg} in the course of EAE in both treated and control mice;

FIG. 9(A) shows the effect of an anti-CD127 antibody (SB/14) of T_{H}17 and T_{H}1 cell counts, but not T_{reg} count, was inhibited when CD127 antibody was added in the onset of differentiation;

FIG. 9(B) shows the effect of an anti-mCD127 antibody (SB/14) as in FIG. 9(A), but on differentiated T_{H}17, but not T_{H}1 or T_{reg};

FIG. 10 shows that addition of IL-7 promoted T_{H}17 expansion/survival and, to a lesser degree, T_{H}1, but not Foxp3 in T_{reg}, when day 9 EAE MOG-specific T cells were cultured;

FIG. 11(A) shows an immunoblot analysis of CD4+ T cells derived ex vivo from treated or control EAE mice showing anti-CD127 antibody treatment changes in signaling pathways related to JAK-STAT and apoptosis as characterized by down-regulation of phosphorylated JAK-1 and phosphorylated STAT-5 and markedly decreased levels of a key pro-apoptotic molecule, BCL-2, and increased activity of an anti-apoptotic molecule, BAX;

FIG. 11(B) shows that anti-CD127 antibody treatment increased the percentage of Annexin-V+ apoptotic cells among CD4+CD127+T cells compared to that of CD4+CD127− T cells derived from treated EAE mice;

FIG. 11(C) shows that differentiated T_{H}17 cells derived from EAE mice undergo apoptosis which can be rescued with IL-7, but this process is slowed if the cells are pre-incubated with an anti-CD127 antibody;

FIG. 11(D) shows that the effects of IL-7 are mediated through the JAK/STAT-5 pathway;

FIG. 12 shows mAb 9B7 and R34.34 have minimal inhibitory effect on the differentiation of T_{H}17 from human total CD4+ cells.

FIG. 13 shows mAb 6C5 inhibition of CD127-ECD binding to immobilised IL-7;

FIG. 14 shows that mAb 6C5 competes with IL-7 for binding to CD127;

FIG. 15 shows that mAb 6C5 and Dendritics antibody R.34.34 compete for binding to CD127;

FIG. 16(A) shows mAb 6A3 inhibition of CD127-ECD binding to immobilised IL-7;

FIG. 16(B) is an inhibition ratio curve of antibodies 6A3, 6C5 and R34.34 at different concentrations of antibody, showing the effect of these antibodies on the binding of CD127-ECD to IL-7;

FIG. 17 shows that mAb 6A3 competes with IL-7 for binding to CD127 expressed on CHO cells;

FIG. 18 shows mAb 6C5 and antibody R.34.34 both inhibit the production of IFNγ by IL-7 stimulated PBMCs;

FIG. 19 shows the ability of antibodies BD, R34.34, 1A11 and 6C5 to block Stat5 signalling induced by IL-7 stimulated PBMCs;

FIG. 20 shows the ability of antibodies BD, R34.34, 1A11 and 6C5 to block Stat5 signalling induced by IL-7 stimulated CCF-CEM cells;

FIG. 21 shows the ability of mAb 6A3 to inhibit IL-17 and IFN-γ production in a T_{H}17 expansion assay;

FIG. 22 shows the inhibitory effect of various anti-CD127 antibodies on the production of IL-17 by hCD4⁺ cells under IL-7 stimulation;

FIG. 23 shows the inhibitory effect of mAb 6A3 on IFN-γ production and IL-17 production by T_{H}17 cells.

### DETAILED DESCRIPTION OF THE INVENTION

The invention is based on the discovery that IL-7/IL-7R signalling is critically required for survival and expansion of committed T_{H}17 cells in both mouse and human systems, while its role in T_{H}17 differentiation is not essential compared to that of IL-6. Surprisingly, the in vivo effect on the immune system by IL-7R antagonism is highly selective in EAE, an animal model for multiple sclerosis, affects T_{H}17 cells and, to a lesser extent, T_{H}1 cells predominantly of the memory phenotype, and spares T_{reg} cells. This selectivity appears to play an important role in rebalancing the ratio of pathogenic T_{H}17 cells and T_{reg} cells by IL-7R antagonism in EAE and is attributable to the treatment efficacy. The novel mechanism of action of IL-7/IL-7R signalling in T_{H}17 cell survival and expansion as discussed above provides a powerful explanation for the treatment efficacy of IL-7R antagonism in EAE and therapeutic implications for human autoimmune diseases, such as MS. IL-7 neutralization or IL-7R antagonism is likely to have unique therapeutic advantages. On one hand, the treatment offers the selectivity that distinguishes pathogenic T_{H}1 and T_{H}17 cells from T_{reg} and unrelated immune cells. On the other hand, additional therapeutic advantages of IL-7R antagonism involve its selective effect on survival and expansion of differentiated T_{H}17 as opposed to T_{H}17 differentiation. It is conceivable that targeting in vivo maintenance of committed T_{H}17 versus T_{H}17 differentiation is more efficacious in a therapeutic context.

Inhibition of IL-7 receptor mediated signalling therefore provides a promising therapeutic intervention for the treatment of autoimmune or inflammatory diseases.

The term IL-7R mediated signalling, as used herein, means the biological effect instigated by the IL-7 receptor complex when bound by its ligand, IL-7. IL-7R mediated signalling therefore includes, but is not necessarily limited to, one or more, or all, of IL-7 induced phosphorylation of STAT-5, IL-7 induced expansion of T_{H}17 cells and IL-7 induced survival of T_{H}17 cells.

### Antagonists

An IL-7 pathway antagonist as used herein is any entity that functionally blocks the biological effects of IL-7, measurable by assays. At the molecular level, one can observe and measure the blocking effect by assays such as IL-7-induced P-STAT5 or Bcl-2. Exemplary p-STAT5 assays are described herein. At the cellular level, one can observe and measure the blocking effect by assays such as Th17 secretion of IL-17 or IFNγ. Exemplary assays are also described herein.

The IL-7/IL-7R pathway antagonists useful in the present invention are capable of inhibiting, partially or in full, phosphorylation of STAT-5 induced by IL-7. STAT-5 phosphorylation can be determined by methods routine in the art, for instance, in an assay such as that described herein (Example 2.3). In such an assay, PBMCs are stimulated with IL-7 in the presence and absence of a test agent. Cells are subsequently assessed quantitatively for the level of pSTAT-5, e.g. by staining for pSTAT-5 (e.g. with a labelled anti-pSTAT-5 antibody) followed by fluorescence activated cell sorting. The levels of phosphorylated STAT-5 could also be determined by ELISA. Those agents which reduce the level of phosphorylated STAT-5 may be potential therapeutic candidates for autoimmune disease.

The antagonist may be capable of reducing levels of phosphorylated STAT-5 by at least 20%, 50%, 75%, 80%, 85%, 90%, 95% or 100% when compared to STAT-5 levels in the absence of the antagonist, or when compared to a negative control, or untreated cells. The antagonist may have an IC₅₀ of 50 μg/ml, 25 μg/ml or less, 10 μg/ml or less, 5 μg/ml or less, or 2 μg/ml or less. In an embodiment, the antagonist has an IC₅₀ of less than or equal to 1 μg/ml, less than or equal to 0.75 μg/ml, less than or equal to 0.5 μg/ml, less than or equal to 0.25 μg/ml, or less than or equal to 0.1 μg/ml.

The antagonists of the invention are particularly effective in inhibiting the expansion of T_{H}17 cells. Expansion of T_{H}17 cells can be determined in a T_{H}17 expansion assay, which comprises stimulating a population of naive T cells to expand in the presence and absence of a test agent, followed by stimulating the cells to produce IL-17 and assessing the level of IL-17 produced by the cells in the presence and absence of the test agent.

In an embodiment, the antagonist is capable of from 20% or more inhibition of IL-17 secretion in such an assay, versus a negative control. More typically, the antagonist is capable of from 50%, from 75%, from 85% or from 90% or more inhibition of IL-17 secretion versus the control. The antagonist may, in some embodiments, exhibit an IC₅₀ of less than or equal to 50 μg/ml in the assay. In other embodiments, the IC₅₀ may be less than or equal to 20 μg/ml, 10 μg/ml or 5 μg/ml.

In an embodiment of this assay, human CD4+ T cells are differentiated into T_{H}17 by stimulation with T cell receptor activation in the presence of IL-1, IL-6, and IL-23. After 5 days of differentiation, CCR6+ cells are sorted out to produce an enriched T_{H}17 population. This population is then stimulated with human IL-7 and the increase in IL-17 and IFN-γ in the supernatant are determined. Blocking the interaction between the IL-7 and CD127 by a functional IL-7/IL-7R pathway antagonist (e.g. an anti-CD127 antibody) in the incubation period should prevent the expansion of the T_{H}17 cells leading to the reduction of IL-17 and IFN-γ production.

In this embodiment, CD4+ T cells may be isolated from human peripheral blood mononuclear cells using a commercial kit (e.g. CD4+ T Cell Isolation Kit II, #130-091-155, Miltenyi Biotec). CD4+ T cells are then typically re-suspended in RPMI medium with 10% FCS at a concentration of 1.5×10E6/ml. Cells are pre-incubated with control or anti-IL-7Rγ antibodies, typically for 30 min. Cells were then cultured with or without 10 ng/ml of IL-7 for 72 h at 37 C. At the end of the incubation, cells are stimulated with 50 ng/ml PMA and 1 ug/ml of lonomycin for 5 h. Cell culture supernatants were then collected and the IL-17 concentration is determined by ELISA (eBiosciences).

### Binding Protein

The isolated binding proteins of the present invention may be in the form of an antibody or immunoglobulin, such as an intact antibody, a human, humanized or chimeric antibody, or fragments or domains of said antibodies. These antibodies of the present invention may comprise one or more, or all of the CDRs found in 9B7 (SEQ ID NOs:4-9), 6C5 (SEQ ID NOs:31-36), 6A5 (SEQ ID NOs:53-58), 1A11 (SEQ ID NOs:73-78) or GR34 (SEQ ID NOs:92-97).

By “binding” in this context it is essentially meant that the binding protein, such as an antibody, binds to (an epitope of) CD127 via an antigen binding domain, and that the binding entails some complementarity between the antigen binding domain and (the epitope of) CD127. A binding protein therefore binds to CD127 or an epitope of CD127 more readily than it would bind to a random, unrelated polypeptide, or a random, unrelated epitope. In other words, there is specificity between the binding protein and (the epitope of) CD127.

The binding proteins of the invention may also be in the form of a soluble CD127 polypeptide.

The binding proteins of the present invention may bind to CD127, such as a monoclonal antibody that specifically binds to CD127. The binding proteins may also be entities that reduce binding of TSLP to the TSLP receptor, and also reduces binding of IL-7 to the IL-7 receptor, for the treatment of multiple sclerosis, such as a bispecific binding protein that binds to the IL-7 and TSLP ligands, or elements of the IL-7R and TSLPR that would give this effect, or combinations of ligands and receptors. In this regard, TSLP antagonists are described in, for example, U.S. Pat. No. 7,304,144 and WO2007096149, and as noted supra, the TSLP receptor comprises CD127. The antagonists of the present invention may therefore be useful as antagonists of TSLP.

### Isolated

The term, “isolated”, as it is used herein, means that the binding proteins are removed from the environment in which they may be found in nature, for example, they may be purified away from substances with which they would normally exist in nature. These binding proteins may be substantially pure, in that the mass of protein in a sample would by constituted of at least 50% or at least 80% binding protein.

### Competition

A binding protein is said to competitively inhibit the binding of a reference binding protein to CD127, to a fragment of CD127 or to an epitope within CD127 if it preferentially binds to that epitope, to the extent that it blocks, to some degree, binding of the reference binding protein to CD127, or to that fragment of CD127 or epitope within CD127. Competitive inhibition may be determined by any method known in the art, for example, competition ELISA assays, surface plasmon resonance (BIAcore), or Scatchard analysis. A binding protein may be said to competitively inhibit the binding of a reference binding protein to a given epitope if the binding of the reference antibody is reduced by at least 90%, at least 80%, at least 70%, at least 60% or at least 50%.

### Intact Antibodies

The binding proteins of the present invention may be “intact antibodies”. Intact antibodies are usually heteromultimeric glycoproteins comprising at least two heavy and two light chains. Aside from IgM, intact antibodies are heterotetrameric glycoproteins of approximately 150 KDa, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions. Each light chain has a variable domain (V_{L}) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fey receptor, half-life/clearance rate via neonatal Fc receptor (FcRn) and complement dependent cytotoxicity via the C1q component of the complement cascade. The human IgG2 constant region has been reported to essentially lack the ability to activate complement by the classical pathway or to mediate antibody-dependent cellular cytotoxicity. The IgG4 constant region has been reported to lack the ability to activate complement by the classical pathway and mediates antibody-dependent cellular cytotoxicity only weakly. Antibodies essentially lacking these effector functions may be termed ‘non-lytic’ antibodies.

### Human Antibodies

The binding proteins of the present invention may be “human antibodies”. Human antibodies may be produced by a number of methods known to those of skill in the art. Human antibodies can be made by the hybridoma method using human myeloma or mouse-human heteromyeloma cell lines see Kozbor J. Immunol 133, 3001, (1984) and Brodeur, Monoclonal Antibody Production Techniques and Applications, pp 51-63 (Marcel Dekker Inc, 1987). Alternative methods include the use of phage libraries or transgenic mice both of which utilize human V region repertories (see Winter G, (1994), Annu. Rev. Immunol 12,433-455, Green L L (1999), J. Immunol. methods 231, 11-23).

Several strains of transgenic mice are now available wherein their mouse immunoglobulin loci has been replaced with human immunoglobulin gene segments (see Tomizuka K, (2000) PNAS 97,722-727; Fishwild D. M (1996) Nature Biotechnol. 14,845-851, Mendez M J, 1997, Nature Genetics, 15,146-156). Upon antigen challenge such mice are capable of producing a repertoire of human antibodies from which antibodies of interest can be selected.

Phage display technology can be used to produce human antibodies (and fragments thereof), see McCafferty; Nature, 348, 552-553 (1990) and Griffiths A D et al (1994) EMBO 13:3245-3260.

### Chimeric and Humanized Antibodies

The binding proteins of the present invention may be “chimeric” or “humanized” antibodies. The use of intact non-human antibodies in the treatment of human diseases or disorders carries with it the now well established problems of potential immunogenicity especially upon repeated administration of the antibody: that is the immune system of the patient may recognise the non-human intact antibody as non-self and mount a neutralising response. In addition to developing fully human antibodies (see above) various techniques have been developed over the years to overcome these problems and generally involve reducing the composition of non-human amino acid sequences in the intact therapeutic antibody whilst retaining the relative ease in obtaining non-human antibodies from an immunised animal e.g. mouse, rat or rabbit. Broadly two approaches have been used to achieve this. The first are chimaeric antibodies, which generally comprise a non-human (e.g. rodent such as mouse) variable domain fused to a human constant region. Because the antigen-binding site of an antibody is localised within the variable regions the chimaeric antibody retains its binding affinity for the antigen but acquires the effector functions of the human constant region and is therefore able to perform effector functions. Chimaeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chain variable regions of the antibody of the invention, e.g. DNA of SEQ ID NO:2 and 3 described supra). The DNA may be modified by substituting the coding sequence for human L and H chains for the corresponding non-human (e.g. murine) H and L constant regions see e.g. Morrison; PNAS 81, 6851 (1984). Thus in another embodiment of the invention there is provided a chimaeric antibody comprising a V_{H} domain having the sequence: SEQ ID NO:2 and a V_{L} domain having the sequence: SEQ ID NO:3 fused to a human constant region (which maybe of a IgG isotype e.g. IgG1).

The second approach involves the generation of humanised antibodies wherein the non-human content of the antibody is reduced by humanizing the variable regions. Two techniques for humanisation have gained popularity. The first is humanisation by CDR grafting. CDRs build loops close to the antibody's N-terminus where they form a surface mounted in a scaffold provided by the framework regions. Antigen-binding specificity of the antibody is mainly defined by the topography and by the chemical characteristics of its CDR surface. These features are in turn determined by the conformation of the individual CDRs, by the relative disposition of the CDRs, and by the nature and disposition of the side chains of the residues comprising the CDRs. A large decrease in immunogenicity can be achieved by grafting only the CDRs of a non-human (e.g. murine) antibody (“donor” antibody) onto a suitable human framework (“acceptor framework”) and constant regions (see Jones of al (1986) Nature 321,522-525 and Verhoeyen M et al (1988) Science 239, 1534-1536). However, CDR grafting per se may not result in the complete retention of antigen-binding properties and it is frequently found that some framework residues of the donor antibody need to be preserved (sometimes referred to as “backmutations”) in the humanised molecule if significant antigen-binding affinity is to be recovered (see Queen C et al (1989) PNAS 86, 10,029-10,033, Co, M et al (1991) Nature 351, 501-502). In this case, human V regions showing the greatest sequence homology (typically 60% or greater) to the non-human donor antibody maybe chosen from a database in order to provide the human framework (FR). The selection of human FRs can be made either from human consensus or individual human antibodies. Where necessary key residues from the donor antibody are substituted into the human acceptor framework to preserve CDR conformations. Computer modelling of the antibody maybe used to help identify such structurally important residues, see WO99/48523.

Alternatively, humanisation maybe achieved by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan E. A. et al; (1991) Mol. Immunol.28, 489-498 and Pedersen J. T. et al (1994) J. Mol. Biol. 235; 959-973). Therefore it is possible to reduce the immunogenicity of a non-human Fv by replacing exposed residues in its framework regions that differ from those usually found in human antibodies. Because protein antigenicity can be correlated with surface accessibility, replacement of the surface residues may be sufficient to render the mouse variable region “invisible” to the human immune system (see also Mark G. E. et al (1994) in Handbook of Experimental Pharmacology vol. 113: The pharmacology of monoclonal Antibodies, Springer-Verlag, pp 105-134). This procedure of humanisation is referred to as “veneering” because only the surface of the antibody is altered, the supporting residues remain undisturbed. Further alternative approaches include that set out in WO04/006955 and the procedure of Humaneering™ (Kalobios) which makes use of bacterial expression systems and produces antibodies that are close to human germline in sequence (Alfenito-M Advancing Protein Therapeutics January 2007, San Diego, Calif.).

It will be apparent to those skilled in the art that the term “derived” is intended to define not only the source in the sense of it being the physical origin for the material but also to define material which is structurally identical to the material but which does not originate from the reference source. Thus “residues found in the donor antibody” need not necessarily have been purified from the donor antibody.

One aspect of the present invention is, therefore, humanized antibodies comprising one or more, or all, of the CDRs found in the mouse antibody 9B7 (SEQ ID NOs: 4-9).

### Multi or Bispecific Antibodies

The binding proteins of the present invention may be “multi-specific” or “bispecific” antibodies. A multispecific or bispecific antibody is an antibody derivative which prevents or reduces binding of both IL-7 and TSLP to their receptors, the antibody having binding specificities for at least two proteins selected from IL-7, TSLP, CD127, IL7R gamma chain or CRLF2, also forms part of the invention. The binding protein of the invention may also have binding specificity for IL-23, which is expressed on the cell surface of T_{H}17 cells, for example, the binding protein may have specificity for both IL-23R (or IL-23) and CD127, or IL-2R (or IL-23) and IL-7.

Methods of making such antibodies are known in the art. Traditionally, the recombinant production of bispecific antibodies is based on the coexpression of two immunoglobulin H chain-L chain pairs, where the two H chains have different binding specificities see Millstein et al, Nature 305 537-539 (1983), WO93/08829 and Traunecker et al EMBO, 10, 1991, 3655-3659. Because of the random assortment of H and L chains, a potential mixture of ten different antibody structures are produced of which only one has the desired binding specificity. An alternative approach involves fusing the variable domains with the desired binding specificities to heavy chain constant region comprising at least part of the hinge region, CH2 and CH3 regions. It is preferred to have the CH1 region containing the site necessary for light chain binding present in at least one of the fusions. DNA encoding these fusions, and if desired the L chain are inserted into separate expression vectors and are then cotransfected into a suitable host organism. It is possible though to insert the coding sequences for two or all three chains into one expression vector. In one preferred approach, the bispecific antibody is composed of an H chain with a first binding specificity in one arm and an H-L chain pair, providing a second binding specificity in the other arm, see WO94/04690. See also Suresh et al Methods in Enzymology 121, 210, 1986.

One potential approach is to produce a bispecific antibody or bispecific fragment such as described supra wherein a first specificity is towards an epitope of IL-7 and a second specificity towards TSLP. Another potential approach is a is to produce a bispecific antibody or bispecific fragment such as described supra wherein a first specificity is towards an epitope of IL-7 and a second specificity towards IL-6.

### Antibody Fragments

The binding proteins of the present invention may be “antibody fragments”. In certain embodiments of the invention there is provided a therapeutic antibody which is an antigen binding fragment. Such fragments may be functional antigen binding fragments of intact and/or humanised and/or chimaeric antibodies such as Fab, Fd, Fab′, F(ab′)₂, Fv, ScFv fragments of the antibodies described supra. The fragments may also be human, camellid or shark or other species, single variable domain antibodies or larger constructs comprising them. Fragments lacking the constant region lack the ability to activate complement by the classical pathway or to mediate antibody-dependent cellular cytotoxicity. Traditionally such fragments are produced by the proteolytic digestion of intact antibodies by, e.g., papain digestion (see for example, WO 94/29348) but may be produced directly from recombinantly transformed host cells. For the production of ScFv, see Bird et al ; (1988) Science, 242, 423-426. In addition, antibody fragments may be produced using a variety of engineering techniques as described below.

Fv fragments appear to have lower interaction energy of their two chains than Fab fragments. To stabilise the association of the V_{H} and V_{L} domains, they have been linked with peptides (Bird et al, (1988) Science 242, 423-426, Huston at al, PNAS, 85, 5879-5883), disulphide bridges (Glockshuber et al, (1990) Biochemistry, 29, 1362-1367) and “knob in hole” mutations (Zhu at al (1997), Protein Sci., 6, 781-788). ScFv fragments can be produced by methods well known to those skilled in the art see Whitlow et al (1991) Methods companion Methods Enzymol, 2, 97-105 and Huston at al (1993) Int. Rev. Immunol 10, 195-217. ScFv may be produced in bacterial cells such as E. Coli but are more typically produced in eukaryotic cells. One disadvantage of ScFv is the monovalency of the product, which precludes an increased avidity due to polyvalent binding, and their short half-life. Attempts to overcome these problems include bivalent (ScFv′)₂ produced from ScFV containing an additional C terminal cysteine by chemical coupling (Adams et al (1993) Can. Res 53, 4026-4034 and McCartney et al (1995) Protein Eng. 8, 301-314) or by spontaneous site-specific dimerization of ScFv containing an unpaired C terminal cysteine residue (see Kipriyanov of al (1995) Cell. Biophys 26, 187-204). Alternatively, ScFv can be forced to form multimers by shortening the peptide linker to between 3 to 12 residues to form “diabodies”, see Holliger et al PNAS (1993), 90, 6444-6448. Reducing the linker still further can result in ScFV trimers (“triabodies”, see Kortt et al (1997) Protein Eng, 10, 423-433) and tetramers (“tetrabodies”, see Le Gall et al (1999) FEBS Lett, 453, 164-168). Construction of bivalent ScFV molecules can also be achieved by genetic fusion with protein dimerizing motifs to form “miniantibodies” (see Pack et al (1992) Biochemistry 31, 1579-1584) and “minibodies” (see Hu et al (1996), Cancer Res. 56, 3055-3061). ScFv-Sc-Fv tandems ((ScFV)₂) may also be produced by linking two ScFv units by a third peptide linker, see Kurucz et al (1995) J. Immol. 154, 4576-4582. Bispecific diabodies can be produced through the noncovalent association of two single chain fusion products consisting of V_{H} domain from one antibody connected by a short linker to the V_{L} domain of another antibody, see Kipriyanov of al (1998), Int. J. Can 77,763-772. The stability of such bispecific diabodies can be enhanced by the introduction of disulphide bridges or “knob in hole” mutations as described supra or by the formation of single chain diabodies (ScDb) wherein two hybrid ScFv fragments are connected through a peptide linker see Kontermann et al (1999) J. Immunol. Methods 226 179-188. Tetravalent bispecific molecules are available by e.g. fusing a ScFv fragment to the CH3 domain of an IgG molecule or to a Fab fragment through the hinge region see Coloma of al (1997) Nature Biotechnol. 15, 159-163. Alternatively, tetravalent bispecific molecules have been created by the fusion of bispecific single chain diabodies (see Alt et al, (1999) FEBS Lett 454, 90-94. Smaller tetravalent bispecific molecules can also be formed by the dimerization of either ScFv-ScFv tandems with a linker containing a helix-loop-helix motif (DiBi miniantibodies, see Muller et al (1998) FEBS Lett 432, 45-49) or a single chain molecule comprising four antibody variable domains (V_{H} and V_{L}) in an orientation preventing intramolecular pairing (tandem diabody, see Kipriyanov et al, (1999) J. Mol. Biol. 293, 41-56). Bispecific F(ab′)2 fragments can be created by chemical coupling of Fab′ fragments or by heterodimerization through leucine zippers (see Shalaby et al, (1992) J. Exp. Med. 175, 217-225 and Kostelny et al (1992), J. Immunol. 148, 1547-1553). Also available are isolated V_{H} and V₁ domains, see U.S. Pat. No. 6, 248,516; U.S. Pat. No. 6,291,158; U.S. Pat. No. 6,172,197.

Other Modifications. The binding proteins of the present invention may comprise other modifications to enhance or change their effector functions. The interaction between the Fc region of an antibody and various Fc receptors (FcγR) is believed to mediate the effector functions of the antibody which include antibody-dependent cellular cytotoxicity (ADCC), fixation of complement, phagocytosis and half-life/clearance of the antibody. Various modifications to the Fc region of antibodies of the invention may be carried out depending on the desired effector property. In particular, human constant regions which essentially lack the functions of a) activation of complement by the classical pathway; and b) mediating antibody-dependent cellular cytotoxicity include the IgG4 constant region, the IgG2 constant region and IgG1 constant regions containing specific mutations as for example mutations at positions 234, 235, 236, 237, 297, 318, 320 and/or 322 disclosed in EP0307434 (WO8807089), EP 0629 240 (WO9317105) and WO 2004/014953. Mutations at residues 235 or 237 within the CH2 domain of the heavy chain constant region (Kabat numbering; EU Index system) have separately been described to reduce binding to FcγRI, FcγRII and FcγRIII binding and therefore reduce antibody-dependent cellular cytotoxicity (ADCC) (Duncan et al. Nature 1988, 332; 563-564; Lund et al. J. Immunol. 1991, 147; 2657-2662; Chappel et al. PNAS 1991, 88; 9036-9040; Burton and Woof, Adv. Immunol. 1992, 51;1-84; Morgan et al., Immunology 1995, 86; 319-324; Hezareh et al., J. Virol. 2001, 75 (24); 12161-12168). Further, some reports have also described involvement of some of these residues in recruiting or mediating complement dependent cytotoxicity (CDC) (Morgan et al., 1995; Xu et al., Cell. Immunol. 2000; 200:16-26; Hezareh et al., J. Virol. 2001, 75 (24); 12161-12168). Residues 235 and 237 have therefore both been mutated to alanine residues (Brett et al. Immunology 1997, 91; 346-353; Bartholomew et al. Immunology 1995, 85; 41-48; and WO9958679) to reduce both complement mediated and FcγR-mediated effects. Antibodies comprising these constant regions may be termed ‘non-lytic’ antibodies.

One may incorporate a salvage receptor binding epitope into the antibody to increase serum half life see U.S. Pat. No. 5,739,277.

Human Fcγ receptors include FcγR (I), FcγRIIa, FcγRIIb, FcγRIIIa and neonatal FcRn. Shields et al, (2001) J. Biol. Chem 276, 6591-6604 demonstrated that a common set of IgG1 residues is involved in binding all FcγRs, while FcγRII and FcγRIII utilize distinct sites outside of this common set. One group of IgG1 residues reduced binding to all FcγRs when altered to alanine: Pro-238, Asp-265, Asp-270, Asn-297 and Pro-239. All are in the IgG CH2 domain and clustered near the hinge joining CH1 and CH2. While FcγRI utilizes only the common set of IgG1 residues for binding, FcγRII and FcγRIII interact with distinct residues in addition to the common set. Alteration of some residues reduced binding only to FcγRII (e.g. Arg-292) or FcγRIII (e.g. Glu-293). Some variants showed improved binding to FcγRII or FcγRIII but did not affect binding to the other receptor (e.g., Ser-267Ala improved binding to FcγRII but binding to FcγRIII was unaffected). Other variants exhibited improved binding to FcγRII or FcγRIII with reduction in binding to the other receptor (e.g. Ser-298Ala improved binding to FcγRIII and reduced binding to FcγRII). For FcγRIIIa, the best binding IgG1 variants had combined alanine substitutions at Ser-298, Glu-333 and Lys-334. The neonatal FcRn receptor is believed to be involved in protecting IgG molecules from degradation and thus enhancing serum half life and the transcytosis across tissues (see Junghans R. P (1997) Immunol. Res 16. 29-57 and Ghetie et al (2000) Annu. Rev. Immunol. 18, 739-766). Human IgG1 residues determined to interact directly with human FcRn include Ile253, Ser254, Lys288, Thr307, Gln311, Asn434 and His435.

The therapeutic antibody of the invention may incorporate any of the above constant region modifications.

In a particular embodiment, the therapeutic antibody essentially lacks the functions of a) activation of complement by the classical pathway; and b) mediating antibody-dependent cellular cytotoxicity. In a more particular embodiment, the present invention provides therapeutic antibodies of the invention having any one (or more) of the residue changes detailed above to modify half-life/clearance and/or effector functions such as ADCC and/or complement dependent cytotoxicity and/or complement lysis.

In a further aspect of the present invention, the therapeutic antibody has a constant region of isotype human IgG1 with alanine (or other disrupting) substitutions at positions 235 (e.g., L235A) and 237 (e.g., G237A) (numbering according to the EU scheme outlined in Kabat). Other derivatives of the invention include glycosylation variants of the antibodies of the invention. Glycosylation of antibodies at conserved positions in their constant regions is known to have a profound effect on antibody function, particularly effector functioning, such as those described above, see for example, Boyd et al (1996), Mol. Immunol. 32, 1311-1318. Glycosylation variants of the therapeutic antibodies of the present invention wherein one or more carbohydrate moiety is added, substituted, deleted or modified are contemplated.

### Analogs

In this context of the present invention, also provided are analogs of the antibodies described. Thus, the invention provides analogs of the R34.34, GR34, 9B7, 6A3, 1A11 or 6C5 CDRs (R34.34 analogs, GR34 analogs, 9B7 analogs, 6A3 analogs, 1A11 analogs or 6C5 analogs). Analogs of a parental antibody (e.g. 6A3 or 9B7) will have the same or similar functional properties to those containing the CDRs of the parental antibody, respectively, in that the 9B7 analog antibodies or 6A3 analog antibodies bind to the same target protein or epitope with the same or similar binding affinity. The analogs may comprise one or more amino acid substitutions within each or all of its CDRs, and in one embodiment at least 75% or 80% of the amino acid residues in the CDRs of the parental antibody are unaltered, in another embodiment at least 90% of the CDRs are unaltered, and in another embodiment at least 95% of the amino acid residues in the CDRs are unaltered. In another embodiment, the CDR H3 of the parental antibody is unaltered in its entirety, whilst the other CDRs may be the same as the corresponding parental antibody CDRs or may be analogs thereof.

### Production Methods

The binding proteins of the present invention may be produced by methods known to the man skilled in the art. Antibodies of the present invention may be produced in transgenic organisms such as goats (see Pollock at al (1999), J. Immunol. Methods 231:147-157), chickens (see Morrow K J J (2000) Genet. Eng. News 20:1-55), mice (see Pollock et al ibid) or plants (see Doran P M, (2000) Curr. Opinion Biotechnol. 11, 199-204, Ma J K-C (1998), Nat. Med. 4; 601-606, Baez J et al, BioPharm (2000) 13: 50-54, Stoger E et al; (2000) Plant Mol. Biol. 42:583-590). Antibodies may also be produced by chemical synthesis. However, antibodies of the invention are typically produced using recombinant cell culturing technology well known to those skilled in the art. A polynucleotide encoding the antibody is isolated and inserted into a replicable vector such as a plasmid for further propagation or expression in a host cell. One useful expression system is a glutamate synthetase system (such as sold by Lonza Biologics), particularly where the host cell is CHO or NSO (see below). Polynucleotide encoding the antibody is readily isolated and sequenced using conventional procedures (e.g. oligonucleotide probes). Vectors that may be used include plasmid, virus, phage, transposons, minichromsomes of which plasmids are a typical embodiment. Generally such vectors further include a signal sequence, origin of replication, one or more marker genes, an enhancer element, a promoter and transcription termination sequences operably linked to the light and/or heavy chain polynucleotide so as to facilitate expression. Polynucleotide encoding the light and heavy chains may be inserted into separate vectors and introduced (e.g. by transformation, transfection, electroporation or transduction) into the same host cell concurrently or sequentially or, if desired both the heavy chain and light chain can be inserted into the same vector prior to such introduction.

### Signal Sequences

Antibodies of the present invention maybe produced as a fusion protein with a heterologous signal sequence having a specific cleavage site at the N terminus of the mature protein. The signal sequence should be recognised and processed by the host cell. For prokaryotic host cells, the signal sequence may be an alkaline phosphatase, penicillinase, or heat stable enterotoxin II leaders. For yeast secretion the signal sequences may be a yeast invertase leader, a factor leader or acid phosphatase leaders see e.g. WO90/13646. In mammalian cell systems, viral secretory leaders such as herpes simplex gD signal and native immunoglobulin signal sequences (such as human Ig heavy chain) are available. Typically the signal sequence is ligated in reading frame to polynucleotide encoding the antibody of the invention.

### Selection Marker

Typical selection genes encode proteins that (a) confer resistance to antibiotics or other toxins e.g. ampicillin, neomycin, methotrexate or tetracycline or (b) complement auxotrophic deficiencies or supply nutrients not available in the complex media or (c) combinations of both. The selection scheme may involve arresting growth of the host cells that contain no vector or vectors. Cells, which have been successfully transformed with the genes encoding the therapeutic antibody of the present invention, survive due to e.g. drug resistance conferred by the co-delivered selection marker. One example is the DHFR-selection system wherein transformants are generated in DHFR negative host strains (eg see Page and Sydenham 1991 Biotechnology 9: 64-68). In this system the DHFR gene is co-delivered with antibody polynucleotide sequences of the invention and DHFR positive cells then selected by nucleoside withdrawal. If required, the DHFR inhibitor methotrexate is also employed to select for transformants with DHFR gene amplification. By operably linking DHFR gene to the antibody coding sequences of the invention or functional derivatives thereof, DHFR gene amplification results in concomitant amplification of the desired antibody sequences of interest. CHO cells are a particularly useful cell line for this DHFR/methotrexate selection and methods of amplifying and selecting host cells using the DHFR system are well established in the art see Kaufman R. J. et al J. Mol. Biol. (1982) 159, 601-621, for review, see Werner R G, Noe W, Kopp K, Schluter M, “Appropriate mammalian expression systems for biopharmaceuticals”, Arzneimittel-Forschung. 48(8):870-80, 1998 August. A further example is the glutamate synthetase expression system (Bebbington et al Biotechnology 1992 Vol 10 p 169). A suitable selection gene for use in yeast is the trp1 gene; see Stinchcomb et al Nature 282, 38, 1979.

### Promoters

Suitable promoters for expressing antibodies of the invention are operably linked to DNA/polynucleotide encoding the antibody. Promoters for prokaryotic hosts include phoA promoter, beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase. Inducible yeast promoters include alcohol dehydrogenase 2, isocytochrome C, acid phosphatase, metallothionein and enzymes responsible for nitrogen metabolism or maltose/galactose utilization.

Promoters for expression in mammalian cell systems include RNA polymerase II promoters including viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40 and non-viral promoters such as EF-1alpha (Mizushima and Nagata Nucleic Acids Res 1990 18(17):5322. The choice of promoter may be based upon suitable compatibility with the host cell used for expression.

### Enhancer Element

Where appropriate, e.g. for expression in higher eukaroytics, additional enhancer elements can be included instead of or as well as those found located in the promoters described above. Suitable mammalian enhancer sequences include enhancer elements from globin, elastase, albumin, fetoprotein, metallothionine and insulin. Alternatively, one may use an enhancer element from a eukaroytic cell virus such as SV40 enhancer, cytomegalovirus early promoter enhancer, polyoma enhancer, baculoviral enhancer or murine IgG2a locus (see W004/009823). Whilst such enhancers are typically located on the vector at a site upstream to the promoter, they can also be located elsewhere e.g. within the untranslated region or downstream of the polyadenylation signal. The choice and positioning of enhancer may be based upon suitable compatibility with the host cell used for expression.

### Polyadenylation/Termination

In eukaryotic systems, polyadenylation signals are operably linked to polynucleotide encoding the antibody of this invention. Such signals are typically placed 3′ of the open reading frame. In mammalian systems, non-limiting example signals include those derived from growth hormones, elongation factor-1 alpha and viral (eg SV40) genes or retroviral long terminal repeats. In yeast systems non-limiting examples of polydenylation/termination signals include those derived from the phosphoglycerate kinase (PGK) and the alcohol dehydrogenase 1 (ADH) genes. In prokaryotic systems polyadenylation signals are typically not required and it is instead usual to employ shorter and more defined terminator sequences. The choice of polyadenylation/termination sequences may be based upon suitable compatibility with the host cell used for expression.

### Other Methods/Elements for Enhanced Yields

In addition to the above, other features that can be employed to enhance yields include chromatin remodelling elements, introns and host-cell specific codon modification. The codon usage of the antibody of this invention thereof can be modified to accommodate codon bias of the host cell such to augment transcript and/or product yield (eg Hoekema A et al Mol Cell Biol 1987 7(8):2914-24). The choice of codons may be based upon suitable compatibility with the host cell used for expression.

### Host Cells

Suitable host cells for cloning or expressing vectors encoding antibodies of the invention are prokaroytic, yeast or higher eukaryotic cells. Suitable prokaryotic cells include eubacteria e.g. enterobacteriaceae such as Escherichia e.g. E. Coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. licheniformis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et al J. Biotechnol. 108 (2004) 185-192), Candida, Trichoderma reesia (EP244, 234), Penicillin, Tolypocladium and Aspergillus hosts such as A. nidulans and A. niger are also contemplated.

Although Prokaryotic and yeast host cells are specifically contemplated by the invention, typically however, host cells of the present invention are vertebrate cells. Suitable vertebrate host cells include mammalian cells such as COS-1 (ATCC No. CRL 1650) COS-7 (ATCC CRL 1651), human embryonic kidney line 293, PerC6 (Crucell), baby hamster kidney cells (BHK) (ATCC CRL. 1632), BHK570 (ATCC NO: CRL 10314), 293 (ATCC NO. CRL 1573), Chinese hamster ovary cells CHO (e.g. CHO-K1, ATCC NO: CCL 61, DHFR minus CHO cell line such as DG44 (Urlaub et al, Somat Cell Mol Genet (1986) Vol 12 pp 555-566), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NSO (see U.S. Pat. No. 5,807,715), Sp2/0, Y0.

Thus in one embodiment of the invention there is provided a stably transformed host cell comprising a vector encoding a heavy chain and/or light chain of the therapeutic antibody as described herein. Typically such host cells comprise a first vector encoding the light chain and a second vector encoding said heavy chain.

Such host cells may also be further engineered or adapted to modify quality, function and/or yield of the antibody of this invention. Non-limiting examples include expression of specific modifying (eg glycosylation) enzymes and protein folding chaperones.

### Cell Culturing Methods.

Host cells transformed with vectors encoding the therapeutic antibodies of the invention may be cultured by any method known to those skilled in the art. Host cells may be cultured in spinner flasks, shake flasks, roller bottles, wave reactors (eg System 1000 from wavebiotech.com) or hollow fibre systems but it is preferred for large scale production that stirred tank reactors or bag reactors (eg Wave Biotech, Somerset, N.J. USA) are used particularly for suspension cultures. Typically the stirred tankers are adapted for aeration using e.g. spargers, baffles or low shear impellers. For bubble columns and airlift reactors direct aeration with air or oxygen bubbles maybe used. Where the host cells are cultured in a serum free culture media this can be supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly vertebrate host cells may utilise a variety of operational modes such as batch, fed-batch, repeated batch processing (see Drapeau et al (1994) cytotechnology 15: 103-109), extended batch process or perfusion culture. Although recombinantly transformed mammalian host cells may be cultured in serum-containing media such media comprising fetal calf serum (FCS), it is preferred that such host cells are cultured in serum—free media such as disclosed in Keen et al (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et al (1995) in Animal Cell technology: Developments towards the 21st century (Beuvery E. C. et al eds), pp 619-623, Kluwer Academic publishers).

Antibodies of the invention secreted into the media may be recovered and purified from the media using a variety of techniques to provide a degree of purification suitable for the intended use. For example the use of therapeutic antibodies of the invention for the treatment of human patients typically mandates at least 95% purity as determined by reducing SDS-PAGE, more typically 98% or 99% purity, when compared to the culture media comprising the therapeutic antibodies. In the first instance, cell debris from the culture media is typically removed using centrifugation followed by a clarification step of the supernatant using e.g. microfiltration, ultrafiltration and/or depth filtration. Alternatively, the antibody can be harvested by microfiltration, ultrafiltration or depth filtration without prior centrifugation. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. In one embodiment, the antibodies of the invention, following various clarification steps, are captured using Protein A or G affinity chromatography followed by further chromatography steps such as ion exchange and/or HA chromatography, anion or cation exchange, size exclusion chromatography and ammonium sulphate precipitation. Typically, various virus removal steps are also employed (e.g. nanofiltration using e.g. a DV-20 filter). Following these various steps, a purified (typically monoclonal) preparation comprising at least 10 mg/ml or greater e.g. 100 mg/ml or greater of the antibody of the invention is provided and therefore forms an embodiment of the invention. Concentration to 100 mg/ml or greater can be generated by ultracentrifugation. Suitably such preparations are substantially free of aggregated forms of antibodies of the invention.

Bacterial systems are particularly suited for the expression of antibody fragments. Such fragments are localised intracellularly or within the periplasma. Insoluble periplasmic proteins can be extracted and refolded to form active proteins according to methods known to those skilled in the art, see Sanchez et al (1999) J. Biotechnol. 72, 13-20 and Cupit P M at al (1999) Lett Appl Microbiol, 29, 273-277.

### Pharmaceutical Compositions

Purified preparations of antibodies of the invention (particularly monoclonal preparations) as described supra, may be incorporated into pharmaceutical compositions for use in the treatment of human diseases and disorders such as those outlined above. Typically such compositions further comprise a pharmaceutically acceptable (i.e. inert) carrier as known and called for by acceptable pharmaceutical practice, see e.g. Remingtons Pharmaceutical Sciences, 16th ed, (1980), Mack Publishing Co. Examples of such carriers include sterilised carrier such as saline, Ringers solution or dextrose solution, buffered with suitable buffers such as sodium acetate trihydrate to a pharmaceutically acceptable pH, such as a pH within a range of 5 to 8. Pharmaceutical compositions for injection (e.g. by intravenous, intraperitoneal, intradermal, subcutaneous, intramuscular or intraportal) or continuous infusion are suitably free of visible particulate matter and may comprise from 1 mg to 10 g of therapeutic antibody, typically 5 mg to 1 g, more specifically 5 mg to 25 mg or 50 mg of antibody. Methods for the preparation of such pharmaceutical compositions are well known to those skilled in the art. In one embodiment, pharmaceutical compositions comprise from 1 mg to 10 g of therapeutic antibodies of the invention in unit dosage form, optionally together with instructions for use. Pharmaceutical compositions of the invention may be lyophilised (freeze dried) for reconstitution prior to administration according to methods well known or apparent to those skilled in the art. Where embodiments of the invention comprise antibodies of the invention with an IgG1 isotype, a chelator of metal ions including copper, such as citrate (e.g. sodium citrate) or EDTA or histidine, may be added to the pharmaceutical composition to reduce the degree of metal-mediated degradation of antibodies of this isotype, see EP0612251. Pharmaceutical compositions may also comprise a solubiliser such as arginine base, a detergent/anti-aggregation agent such as polysorbate 80, and an inert gas such as nitrogen to replace vial headspace oxygen.

Effective doses and treatment regimes for administering the antibody of the invention are generally determined empirically and are dependent on factors such as the age, weight and health status of the patient and disease or disorder to be treated. Such factors are within the purview of the attending physican. Guidance in selecting appropriate doses may be found in e.g. Smith et al (1977) Antibodies in human diagnosis and therapy, Raven Press, New York.

### Clinical Uses

The antagonists of the present invention may be used in the therapy of multiple sclerosis and in other autoimmune or inflammatory diseases, particularly those in which pathogenic T_{H}17 cells are implicated. Such diseases are associated with high levels of IL-17 expression. Elevated levels of IL-17 have been reported in serum and CSF of MS patients (Matusevicius, D. et al.; Mult. Scler. 5, 101-104; 1999) and in the synovial fluid obtained from rheumatoid arthritis patients. IL-17 has also been implicated in psoriasis (Homey et al.; J. Immunol. 164(12):6621-32; 2000), while Hamzaoui et al reported high levels of IL-17 in Behcet's disease (Scand. J. Rhuematol.; 31:4, 205-210; 2002). Elevated IL-17 levels have also been observed in systemic lupus erythrematosus (SLE) (Wong et al.; Lupus 9(8):589-93; 2000).

Inhibition of IL-7 receptor mediated signalling may also be useful in the treatment of inflammatory (non-autoimmune) diseases in which elevated IL-17 has been implicated, such as asthma.

Accordingly, inflammatory and/or autoimmune diseases of the invention include inflammatory skin diseases including psoriasis and atopic dermatitis; systemic scleroderma and sclerosis; inflammatory bowel disease (IBD); Crohn's disease; ulcerative colitis; ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); dermatitis; meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; psoriatic arthritis; neuromyelitis optica, Guillain-Barre syndrome (GBS), COPD, type 1 diabetes, etc.

In particular, the antagonists of the present invention may be useful in the therapy of multiple sclerosis, in all its forms, including neuromyelitis optica. Treatment with an antagonist of the present invention is predicted to be most efficacious when administered in the context of active inflammatory disease, i.e. when used in the treatment of clinically isolated syndrome or relapsing forms of MS. These stages of disease can be defined clinically and/or by imaging criteria such as gadolinium enhancement or other more sensitive techniques, and/or other as yet undefined biomarkers of active disease. Particularly, the antagonists of the invention can be used to treat RRMS (via intravenous, sub-cutaneous, oral or intramuscular delivery) when the patients are entering or are in relapse. In an embodiment, the antagonist of the invention is administered to the patient at the onset of relapse, or within 1 hr, 2 hrs, 3 hrs, 6 hrs, 12 hrs, 24 hrs, 2 days, 3 days, 4 days, 5 days, 6 days, 7 days, 8 days, 9 days or 10 days from the onset of relapse.

The use of biomarkers such as CD127 expression and intracellular cytokine staining (e.g. IL-17 staining) provides criteria for applying the therapeutic anti-CD127 binding protein. A subgroup of MS patients with increased T_{H}17 in their CD4+ T cells are primary candidates for the treatment. In one embodiment the methods of treatment are methods of the present invention are methods to treat those patients that express high level of CD127 on their T cells, making them susceptible to anti-CD127 treatment. Treatment with anti-CD127 may likely shorten the time of relapse and quicken the attenuation of the clinical activities measurable by EDSS or MRI. Once the patients enter remission, the treatment may be stopped to avoid complications such as the inhibition of normal T cell development and homeostasis. The use of the anti-CD127 antibody may also prolong the period between relapses and improve patients' quality of life.

Unless defined otherwise, all technical and scientific terms used herein have the same meaning as commonly used and understood by those of ordinary skill in the art.

The examples, and the materials used therein are for the purposes of illustration only and are not intended to be limiting.

| Description | SEQ ID NO: |
|---|---|
| Human CD127 amino acid sequence | 1 |
| 9B7 Heavy chain variable region | 2 |
| 9B7 Light chain variable region | 3 |
| 9B7 CDR H1 | 4 |
| 9B7 CDR H2 | 5 |
| 9B7 CDR H3 | 6 |
| 9B7 CDR L1 | 7 |
| 9B7 CDR L2 | 8 |
| 9B7 CDR L3 | 9 |
| Epitope of SB/14 (171-187 mouse CD127) | 10 |
| Epitope of A7R34 (80-95 mouse CD127) | 11 |
| 9B7 Heavy chain FR1 sequence | 12 |
| 9B7 Heavy chain FR2 sequence | 13 |
| 9B7 Heavy chain FR3 sequence | 14 |
| 9B7 Heavy chain FR4 sequence | 15 |
| 9B7 Light chain FR1 sequence | 16 |
| 9B7 Light chain FR2 sequence | 17 |
| 9B7 Light chain FR3 sequence | 18 |
| 9B7 Light chain FR4 sequence | 19 |
| 9B7 epitope by peptide ELISA | 20 |
| 9B7 epitope by peptide ELISA | 21 |
| 9B7 epitope by peptide ELISA | 22 |
| 9B7 Phage epitope region 1 | 23 |
| 9B7 Phage epitope region 2 | 24 |
| 9B7 Phage epitope region 3 | 25 |
| 9B7 Consensus epitope region 1 | 26 |
| 9B7 Consensus epitope region 2 | 27 |
| 9B7 Consensus epitope region 3 | 28 |
| 6C5 Heavy chain variable region | 29 |
| 6C5 Light chain variable region | 30 |
| 6C5 CDR H1 | 31 |
| 6C5 CDR H2 | 32 |
| 6C5 CDR H3 | 33 |
| 6C5 CDR L1 | 34 |
| 6C5 CDR L2 | 35 |
| 6C5 CDR L3 | 36 |
| 6C5 Heavy chain FR1 sequence | 37 |
| 6C5 Heavy chain FR2 sequence | 38 |
| 6C5 Heavy chain FR3 sequence | 39 |
| 6C5 Heavy chain FR4 sequence | 40 |
| 6C5 Light chain FR1 sequence | 41 |
| 6C5 Light chain FR2 sequence | 42 |
| 6C5 Light chain FR3 sequence | 43 |
| 6C5 Light chain FR4 sequence | 44 |
| 6C5 Epitope by BIAcore | 45 |
| 6C5 Phage epitope region 1 | 46 |
| 6C5 Phage epitope region 2 | 47 |
| 6C5 Consensus epitope region 1 | 48 |
| 6C5 Consensus epitope region 2 | 49 |
| 6C5 Consensus epitope region 3 | 50 |
| 6A3 Heavy chain variable region | 51 |
| 6A3 Light chain variable region | 52 |
| 6A3 CDR H1 | 53 |
| 6A3 CDR H2 | 54 |
| 6A3 CDR H3 | 55 |
| 6A3 CDR L1 | 56 |
| 6A3 CDR L2 | 57 |
| 6A3 CDR L3 | 58 |
| 6A3 Heavy chain FR1 sequence | 59 |
| 6A3 Heavy chain FR2 sequence | 60 |
| 6A3 Heavy chain FR3 sequence | 61 |
| 6A3 Heavy chain FR4 sequence | 62 |
| 6A3 Light chain FR1 sequence | 63 |
| 6A3 Light chain FR2 sequence | 64 |
| 6A3 Light chain FR3 sequence | 65 |
| 6A3 Light chain FR4 sequence | 66 |
| 6A3 Phage epitope region 1 | 67 |
| 6A3 Phage epitope region 2 | 68 |
| 6A3 Phage epitope region 3 | 69 |
| 6A3 Phage epitope region 4 | 70 |
| 1A11 Heavy chain variable region | 71 |
| 1A11 Light chain variable region | 72 |
| 1A11 CDR H1 | 73 |
| 1A11 CDR H2 | 74 |
| 1A11 CDR H3 | 75 |
| 1A11 CDR L1 | 76 |
| 1A11 CDR L2 | 77 |
| 1A11 CDR L3 | 78 |
| 1A11 Heavy chain FR1 sequence | 79 |
| 1A11 Heavy chain FR2 sequence | 80 |
| 1A11 Heavy chain FR3 sequence | 81 |
| 1A11 Heavy chain FR4 sequence | 82 |
| 1A11 Light chain FR1 sequence | 83 |
| 1A11 Light chain FR2 sequence | 84 |
| 1A11 Light chain FR3 sequence | 85 |
| 1A11 Light chain FR4 sequence | 86 |
| 1A11 Phage epitope region 1 | 87 |
| 1A11 Phage epitope region 2 | 88 |
| 1A11 Phage epitope region 3 | 89 |
| GR34 Heavy chain variable region | 90 |
| GR34 Light chain variable region | 91 |
| GR34 CDR H1 | 92 |
| GR34 CDR H2 | 93 |
| GR34 CDR H3 | 94 |
| GR34 CDR L1 | 95 |
| GR34 CDR L2 | 96 |
| GR34 CDR L3 | 97 |
| GR34 Heavy chain FR1 sequence | 98 |
| GR34 Heavy chain FR2 sequence | 99 |
| GR34 Heavy chain FR3 sequence | 100 |
| GR34 Heavy chain FR4 sequence | 101 |
| GR34 Light chain FR1 sequence | 102 |
| GR34 Light chain FR2 sequence | 103 |
| GR34 Light chain FR3 sequence | 104 |
| GR34 Light chain FR4 sequence | 105 |
| R.34.34 Epitope by BIAcore region 1 | 106 |
| R.34.34 Epitope by BIAcore region 2 | 107 |
| R.34.34 Epitope by BIAcore region 3 | 108 |
| R.34.34 Epitope by BIAcore region 4 | 109 |
| R.34.34 Epitope by BIAcore region 5 | 110 |
| R.34.34 Phage epitope region 1 | 111 |
| R.34.34 Phage epitope region 2 | 112 |
| R.34.34 Phage epitope region 3 | 113 |
| R.34.34 Consensus epitope region 1 | 114 |
| R.34.34 Consensus epitope region 2 | 115 |
| R.34.34 Consensus epitope region 3 | 116 |
| CD127 Epitope region 1 | 117 |
| CD127 Epitope region 2 | 118 |
| CD127 Epitope region 3 | 119 |
| CD127 Epitope region 4 | 120 |
| CD127 Epitope region 5 | 121 |
| CD127 Epitope region 1a | 122 |
| CD127 Epitope region 2a | 123 |
| CD127 Epitope region 3a | 124 |
| CD127 Epitope region 4a | 125 |
| CD127 Epitope region 5a | 126 |

### EXAMPLE 1

### Characterization of Monoclonal Antibodies that Bind to Mouse CD127

### Methods

1.1 Evaluation of Commercially Available Mouse Antibodies to Mouse CD127 by Using FACS on pStat5 Detection Assay

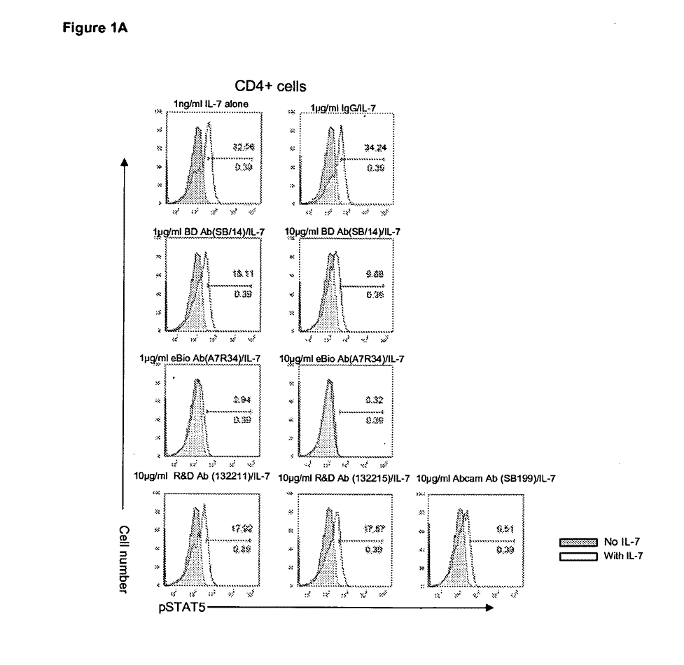

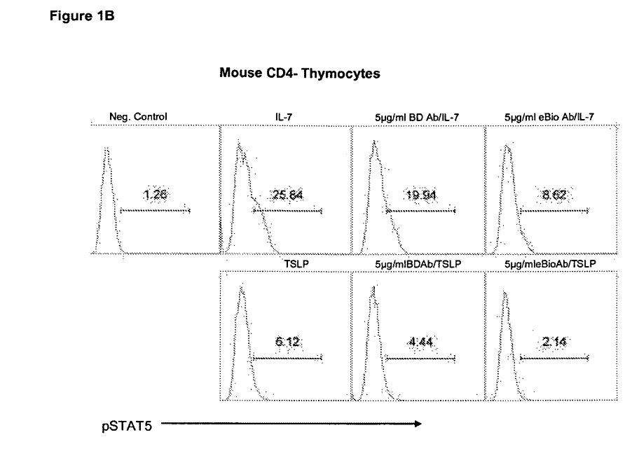

In this example, we identified commercially available anti-mouse CD127 antibodies that inhibited IL-7-induced Stat5 phosphorylation (pStat5). Briefly, splenocytes were prepared from C57B/6 mouse spleens by a standard protocol; CD4+ T cells were then purified from the splenocytes using a Miltenyi magnetic isolation kit (Cat#130-049-201); one million CD4⁺ T cells per ml were first incubated with the indicated antibodies and concentrations as shown in the figure below for 30 min. at 37° C.; the antibodies used were BD Biosciences control rat IgG2a (#553926), BD Biosciences anti-CD127 (Clone SB/14, #550426), eBiosciences anti-CD127 (Clone:A7R34, #16-1271), Abcam anti-CD127 (Clone SB199, #ab36428), R&D anti-CD127 (MAB7471 and 7472) ; cells were then either untreated or treated with 1 ng/ml mouse IL-7 for 60 min. at 37° C.; cells were collected and immediately put on ice after the IL-7 treatment; cells were then washed with ice-cold PBS once and fixed in 1% paraformaldehyde for 10 min. at 37° C.; cells were washed with PBS and incubated with 500 μl 90% methanol/PBS for 30 min. on ice; cells were washed again in PBS and cell pellets were resuspended in 100 ul PBS; cells were stained by 5 ul of anti-pStat5-Alexa Fluor 647 antibody (BD Biosciences, #612599) for 1 h in RT in the dark; cells were then washed two times with PBS and analyzed by flow cytometry with a BD Biosciences Facscalibur machine according to the manufacturer's instructions. The results are shown in FIG. 1.

In the graphs, cell numbers were plotted against mean fluorescence intensity (MFI) of intracellular pStat5. The histograms showed the MFI of untreated CD4⁺ T cells. Treatment with IL-7 shifted the MFI to the right and cells with increased pStat5 were defined by the appropriate gate as shown by the bar in the histogram. The control IgG did not inhibit pStat5. However, A7R34 strongly inhibited pStat5. The antibody clone SB/14 also showed inhibition although it was not as strong as A7R34. The abcam clone SB199 and the R&D systems antibodies could only inhibit Stat5-p partially at high concentration.

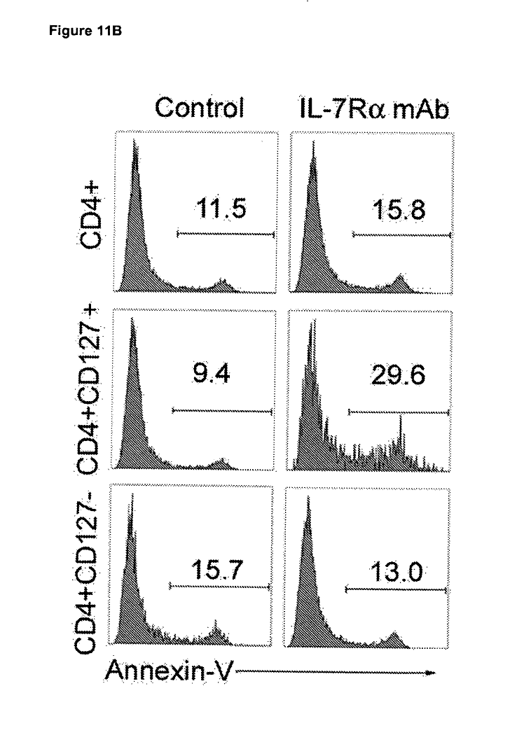

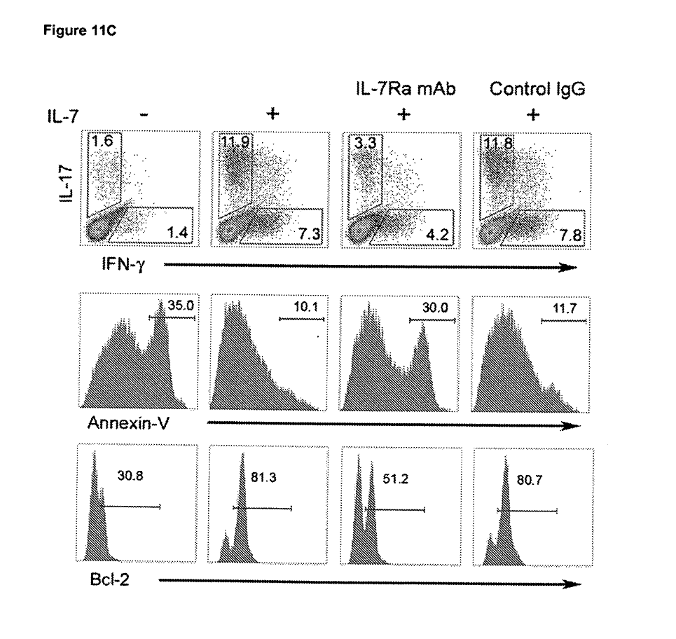

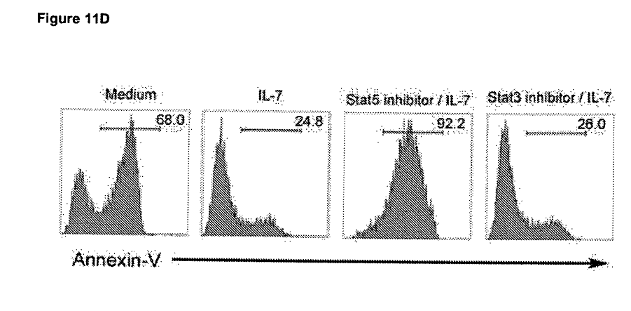

SB/14 was also tested for its inhibition of IL-7-driven expansion of differentiated T_{H}17 in vitro. Experimental autoimmune encephalomyelitis (EAE) was induced in mice by immunization of myelin oligodendrocyte glycoprotein (MOG) as described in Example 3 below. CD4+ T cells were harvested from the spleens or lymph nodes of EAE mice and were cultured in vitro in the absence or presence of IL-7 for 3 days. As shown in FIG. 11C, IL-7 promoted the expansion of T_{H}17 cells, detectable by IL-17 intracellular staining. Antibody SB/14 against mouse IL-7Ra but not a control IgG inhibited the IL-7-driven expansion of Th17 cells.

The antibodies were also tested for the inhibition of TSLP-mediated pStat5 in mouse thymocytes. CD4− cells in thymocytes expressed functional TSLP receptors and were gated in the FACS analysis. As shown in FIG. 1B, IL-7-induced and TSLP-induced pStat5 was inhibited by SB/14 (BD) and A7R34 (eBio). Therefore, antibodies against mouse CD127 (SB/14 and A7R34) inhibited both IL-7-mediated and TSLP-mediated signalling.

### 1.2 Identification of Epitope by Peptide ELISA

15 mers with 7 overlapping peptides of mouse IL7RECD were synthesized by Shanghai Science peptide Biology Technology, and by GL Biochem (Shanghai) Ltd. All peptides were prepared by continuous-flow solid phase peptide synthesis. The peptides were then biotinylated at the N-terminus of the peptide with a spacer Acp between the peptide and the biotin moiety, i.e., biotin-Acp-peptide.

The wells of a 96-well plate with 100 uL of 1 ug/mL each testing antibody in carbonate buffer (15 mM Na₂CO₃, 35 mM NaHCO₃, 0.2 g/L NaN₃, at pH 9.6) was coated for overnight at 4° C. Next day plate was washed three times 200 μl/well with wash buffer (1× PBS containing 0.05% Tween-20) and 200 μl/well blocking buffer (10 mg/ml bovine serum albumin (BSA) in PBST) was incubated for 1 hour at 37° C. After washing the plate three times, 100 uL of 2 ug/mL synthetic biotinylated peptide was applied at 37° C. for 1 hour. After three washes, 100 uL/well of 1/2000 diluted HRP-SA was added and incubated at 37° C. for 30 minutes. 100 uL/well of TMB substrate solution was used after five washes. Incubation was 2 to 5 min at RT before stopped with 2N HCl. Read plate at 450 nm with an appropriate time-resolved plate reader.

### 1.3 Prediction of Epitopes by Biopanninq Phage Peptide Display

Random peptide libraries displayed on filamentous bacteriophage M13 has been used as a tool to map the epitopes of monoclonal antibodies (Scott and Smith, 1990, searching for peptide ligands with an epitope library, Science, 249:386-390). We have used a commercial phage-displayed random peptide library and an in house phage-displayed random peptide library to identify phage peptides that bind to mouse antibody. Enriched phage displayed peptide consensus sequences or mimotopes from identified phage peptides were employed to predict possible epitopes of mouse antibody (phage peptide mimotopes: antibody interaction site on the surface of the antigen mimicked by phage peptide or a mimic of an epitope) (Geysen et al., 1986, a priori delineation of a peptide which mimics a discontinuous antigenic determinant. Mol. Immunol., 23:709-715; Luzzago et al., 1993, mimicking of discontinuous epitopes by phage-displayed peptides, I. Epitope mapping of human H ferritin using a phage library of constrained peptides, Gene, 128: 51-57). Phage peptide mimotopes identified from the 2 random libraries predicted 2 possible discontinuous epitopes of mouse antibody.

### Random Peptide Libraries:

1. Ph.D-12 phage displayed random peptide library (from New England Biolabs Inc., #E8110S)
2. fGWX10 phage displayed random peptide library (GSK in house library)

### Biopanning Procedure Using Ph.D-12 Phage Displayed Random Peptide Library:

Biopanning of Ph.D-12 phage displayed random peptide library against immobilized mAb 9B7 was conducted essentially according to the manufacturer's instruction manual. Briefly:
1) Coat 100 μg/ml of each testing antibody (in 0.1 M NaHCO₃, pH 8.6) to the well of the 12-well plate and incubate overnight at 4° C. with gentle agitation.
2) Incubate with blocking Buffer (0.1 M NaHCO₃, pH8.6, 5 mg/ml BSA, 0.02 NaN₃ was used for the anti-mouse antibody procedures; 1% milk was subsequently used for the anti-human antibody procedures) for 1 hour at 4° C. and then TBST wash for six times (TBS+0.1% [v/v] Tween-20).
3) Apply diluted 4×10¹⁰ phage in TBST onto coated plate and rock gently for 60 minutes at room temperature.
4) Discard nonbinding phage and wash plates 10 times with TBST.
5) Elute bound phage with 300 ul 0.2 M Glycine-HCl (pH 2.2), 1 mg/ml BSA and neutralize with 45 μl 1 M Tris-HCl (pH 9.1) for further two rounds of biopanning.
6) Add the eluate to the inoculated E. coli. ER2738 culture and incubate at 37° C. with vigorous shaking for 4.5 hours. Centrifuged cultured supernatant then precipitate in PEG/NaCl at 4° C. for overnight.
7) Titer the resulting third round amplified eluate on LB/IPTG/Xgal plates. Plaques from titering plates were used for DNA sequencing.
   The Biopanning Procedure Using fGWX10 Phage Displayed Random Peptide Library:
   The in house phage library, fGWX10, displaying 10-mer random peptide sequences was constructed as described previously (Deng et al., 2004, Identification of peptides that inhibit the DNA binding, trans-activator, and DNA replication functions of the human papillomavirus type 11 E2 protein, J. Virol., 78: 2637-2641). Briefly,
1) Coat 100 μg/ml of each testing antibody (in 0.1 M NaHCO₃, pH 8.6) to the well of the 12-well plate and incubate overnight at 4° C. with gentle agitation.
2) Inoculate one tube with 10 ml LB medium with E. coli K91. Incubate culture at 37° C. with vigorous shaking.
3) Incubate with blocking Buffer (0.1 M NaHCO₃, pH8.6, 5 mg/ml BSA, 0.02 NaN₃ was used for the anti-mouse antibody procedures; 1% milk was subsequently used for the anti-human antibody procedures) for 1 hour at 4° C. and then TBST wash for six times (TBS+0.1% [v/v] Tween-20).
4) Apply diluted 50 ul fGWX10 phage (diversity 1×10¹⁰) with 350 ml of TBST onto coated plate and rock gently for 60 minutes at room temperature and wash plates 10 times with TBST
5) Elute bound phage with 300 ul 0.2 M Glycine-HCl (pH 2.2), 1 mg/ml BSA into a microcentrifuge tube and neutralize with 45 μl 1 M Tris-HCl, pH 9.1 for further two rounds of biopanning.
6) Titer the unamplified third round eluate using inoculated E. coli K91 cells on LB/Tet plates. Colony from titering was used for DNA sequencing. Store the remaining eluate at 4° C.

### 1.4 Determination of Epitope Binding of Mouse Antibody by Biacore

The binding epitope of anti-mouse CD127 antibodies for mouse CD127 were assessed using a Biacore T100 system (GE Healthcare). Briefly, anti-mouse CD127 antibodies was immobilized on a CM5 biosensor chip with a final level of ˜100 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was activated/deactivated using EDC/NHS/ethanol amine. 15 mers with 7 overlapping peptides of IL7R ECD were synthesized by Shanghai Science peptide Biology Technology, and by GL Biochem (Shanghai) Ltd. Each peptide was injected at various concentrations for 120s at a flow rate of 30 uL/min. Kd values were calculated using the Biacore evaluation software package. The runs were carried out at 25° C.

Table 1 shows epitope regions of mouse CD127 (NP_{—}032398) for two mouse antibodies, BD Biosciences Clone SB/14 and eBiosciences Clone A7R34 that identified by one or more of the methods phage peptide library, peptide ELISA and Biacore

TABLE 1
Summary of epitope studies for anti-murine
CD127 antibodies SB/14 and A7R34

| | | | Phage | Pep- |
|---|---|---|---|---|
| Anti- | | | li- | tide |
| body | Mouse epitope | BIAcore | brary | ELISA |
| SB/14 | 171-PARGESNWTHVSLFHTR-187 | ✓ | ✓ | — |
| | (SEQ ID NO: 10) | | | |
| A7R34 | 80-VKCLTLNKLQDIYFIK-95 | N/D | ✓ | N/D |
| | (SEQ ID NO: 11) | | | |

### EXAMPLE 2

### Generation of Monoclonal Antibodies that Bind to Human CD127 (hCD127)

Monoclonal antibodies (mAbs) were produced by hybridoma cells generally in accordance with the method set forth in E Harlow and D Lane, Antibodies a Laboratory Manual, Cold Spring Harbor Laboratory, 1988.

Antigen used to generate hybridomas including 9B7 and 6C5 was a dimeric recombinant human CD127 extracellular domain (ECD)-Fc (R&D Systems #306-IR), comprising amino acid 21-262 of human CD127 (SEQ ID No:1). Antigen used to generate hybridomas including 6A3 and 1A11 was a construct containing the full ECD of CD127 (amino acids 21-219 of SEQ ID NO:1).

Balb/c mice were primed and boosted by intraperitoneal injection with Antigen in FCA or FIA (Sigma-Aldrich, #F5881, #F5506) (1:1; vol:vol). Spleens from responder animals were harvested and fused to SP/0 myeloma cells to generate hybridomas. Hybridomas of interest were monocloned using semi-solid media (methyl cellulose solution) and manually picked up into 96-well plate. The hybridoma supernatant material was screened for binding to CD127ECD using ELISA, CHO-CD127 transfected cell FACS, pStat5 FACS and BIAcore T100 (results shown below).

Selected purified mAbs (isolated from hybridoma supernatants 9B7, 6C5, 6A3 and 1A11) were tested for inhibition of IL-7 induced IFN-γ and IL-17 in a T_{H}17 expansion assay. In addition, commercially available anti-hCD127 R34.34 was shown to inhibit IL-7 induced IFN-γ and IL-17 in a T_{H}17 expansion assay and was also selected for further analysis.

### Methods

### 2.1 Selection of Hybridoma Binding to CD127 by ELISA

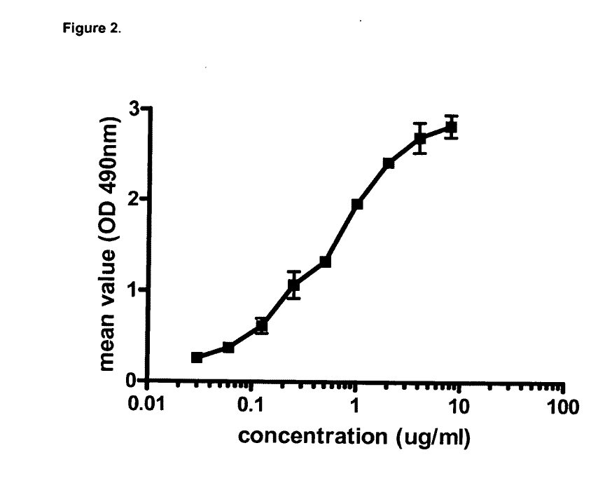

5 μg/ml recombinant human CD127ECD was coated onto an ELISA plate. Anti-CD127 antibodies from the test hybridoma supernatants or purified material were titrated across the plate. The level of binding was detected by treatment with a horse-radish peroxidase (HRP) -conjugated goat-anti-mouse IgG antibody. The ELISA was developed using TMB substrate. Results for the 9B7 hybridoma supernatant are shown in FIG. 2.

### 2.2 Fluorescence Activated Cell Sorting (FACS) Analysis

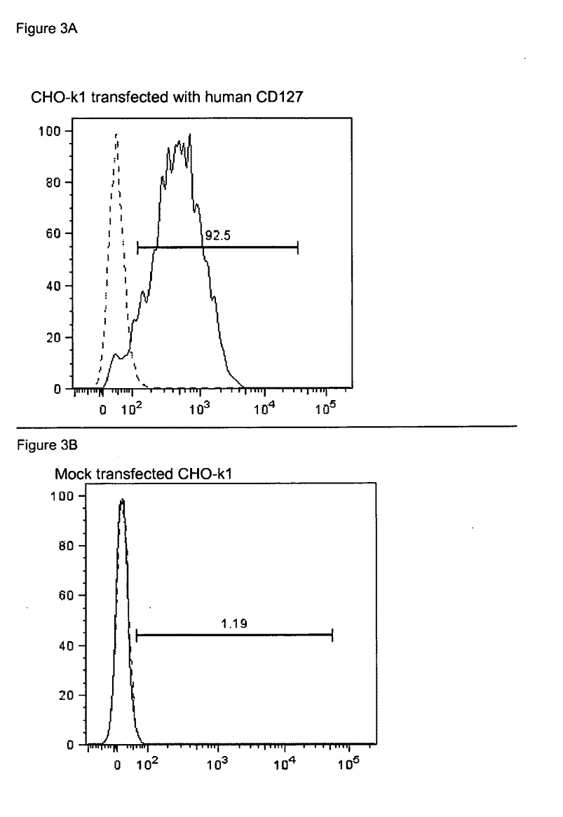

Mock transfected CHO or CHO-CD127 cells (2×10⁶ cells/ml) were stained with hybridoma supernatants or purified antibodies at 1 μg/ml for 1 hour with 4% FCS in PBS (FACS buffer). Cells were also stained in a suitable negative control mouse antibody and anti-human CD127 positive control (R34.34 Dendritics Inc. #DDX0700). Cells were washed in FACS buffer and then stained with an anti-mouse IgG ALEXA488 secondary antibody 1:2000 (Invitrogen Inc. #13-A11017). After washing in FACS buffer, cells were analysed in LSR II (BD Biosciences Inc.). Results for the 9B7 antibody are shown in FIG. 3.

2.3 Inhibition of IL7 Stimulated IL7 receptor signalled Stat5 Phosphorvlation by 9B7

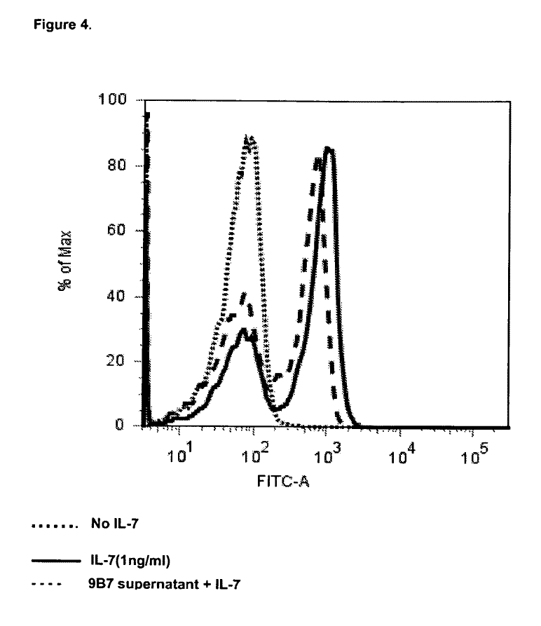

Defrost Frozen PBMCs the night before the experiment, and leave them in RPMI 1640 medium containing 10% of FBS for recovering. For screening functional antibody to CD127, hybridoma culture medium, positive control antibody (R34.34, Dendritics Inc) at 2 ug/ml and 0.2 ug/ml, or testing supernatant samples were incubated with 5×10⁵ PBMC cells for 30 mins before stimulating with 1 ng/ml of IL-7. The untreated cells were analyzed as the background signal, while IL-7 treated cells were set as negative control. After 30 mins' incubation with the controls or testing samples, the cells were stimulated with 1 ng/ml of IL-7 for 15 mins at 37° C. Cells then were fixed 1.6% of paraformaldehyde/PBS for 10 min at 37° C. and were permeabilized in 100% methanol for 20-30 mins. Cells then were washed twice in stain buffer (1% BSA in PBS) and stained with 7 ul of Alexa-647 labelled anti-pStat5 antibody (BD Biosciences Inc #612599) for 1 hr. Samples were analyzed on BD LSR II FACS instrument. Results for 9B7 are shown in FIG. 4.

An optimised process was used to utilised for antibodies R34.34, 6A3, 1A11 and 6C5, as described in Section 3.19.

### 2.4 Inhibition of IL-7-Induced IL-17 Production in Human Th17 Expansion Assay

Memory T_{H}17 cells in a population of normal human CD4+T cells are stimulated to expand for three days. These T_{H}17 cells are then activated by PMA and ionomycin to stimulate the production of IL-17. Blocking the interaction between the IL-7 and CD127 by a functional anti-CD127 antibody in the three day incubation period should prevent the expansion of the T_{H}17 cells leading to the reduction of IL-17 production.

CD4+T cells were isolated from human peripheral blood mononuclear cells using a commercial kit (CD4+ T Cell Isolation Kit II, #130-091-155, Miltenyi Biotec). CD4+ T cells were resuspended in RPMI medium with 10% FCS at a concentration of 1.5×10E6/ml. Cells were pre-incubated with control or anti-IL-7Rα antibodies for 30 min. Cells were then cultured with or without 10 ng/ml of IL-7 for 72 h at 37 C. At the end of the incubation, cells were stimulated with 50 ng/ml PMA and 1 ug/ml of lonomycin for 5 h. Cell culture supernatants were then collected and the IL-17 concentration were determined by Elisa (eBiosciences). This assay was utilised for antibody 9B7.

Antibodies 6C5, 6A3 and R34.34 were assayed according to the following protocol. CD4+cells were isolated according to the manual (#130-091-155, Miltenyi). Approximately 1×10⁶/ml of the CD4+ cells in 100 μl were mixed with equal volume of 2× Th17 differentiation medium (2 μg/ml anti-CD28+10μg/ml anti-IFN-γ+10 μg/ml anti-IL-4+12.5 ng/ml IL-1β+20 ng/ml IL-23+50 ng/ml IL-6) and cultured in 37° C. with 5% CO₂ for 5 days. Treatment by the various cytokines and growth factors in the T_{H}17 medium preferentially differentiated the CD4+ cells into T_{H}17 cells. CCR6+ cells from the differentiated cultured cells at day 5 were sorted using BD FACS SORP Aria II. The CCR6+ cells were then adjusted to 2×10⁶/ml for the IL-17 production assay.

To measure IL-17 and IFN-γ level, 100 μl of CCR6+cells were pre-incubated with testing antibody for 1 h at 37° C., and then mixed with 100 μl of 10 ng/ml IL-7. The cells were cultured for 24-40 hours in 37° C. with supplement of 5% CO₂. IFN-γ and IL-17 levels in 100 ul of culture supernatant were measured by FlowCytomix (Bender MedSystems) at 24 h and 40 h, respectively.

### 2.5 Determination of Kinetics of Binding by Surface Plasmon Resonance

The binding kinetics of anti-CD127 antibodies for human CD127 was assessed using a Biacore T100 system (GE Healthcare). Briefly, recombinant human CD127 ECD was immobilized on a CM5 biosensor chip with a final level of ˜100 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was activated/deactivated using EDC/NHS/ethanol amine. Analytes (anti-CD127 antibodies) were injected at various concentrations for 120 s at a flow rate of 30 uL/min. The antigen surfaces were regenerated with 10 mM Glycine-HCl, pH2.5. Kd values were calculated using the Biacore evaluation software package. The runs were carried out at 25° C.

TABLE 2
Kinetic data for supernatant material of 9B7.
The run was carried out at 37° C.

| Antibody | Ka | Kd | KD (M) |
|---|---|---|---|
| 9B7 | 8.09E+04 | 4.50E−05 | 5.56E−10 |

The isotype of 9B7 was determined to be IgG1 with a kappa light chain constant region.

The following assay was used to evaluate the binding kinetics of anti-CD127 antibodies 6C5, 6A3, 1A11 and GR34. Antibody kinetics were assessed using a Biacore T100 system (GE Healthcare) with a reaction temperature of 25° C. Rabbit anti-mouse IgG antibody was immobilized on a CM5 biosensor chip with a final level of -10000 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was blank immobilized using EDC/NHS/ethanol amine. For ligand capturing, 25 nM of 6C5 was injected over the chip surface for 30 s at 10 μL/min. Analytes (recombinant human CD127 ECD) were then injected at various concentrations for 500 s at 30 μL/min. The sensor chip surfaces were regenerated with 10 mM Glycine-HCl, pH 1.7. Kd values were calculated using the Biacore evaluation software package.

TABLE 3
Kinetics data for 6C5 and 6A3

| | ka (1/Ms) | kd (1/s) | KD (M) |
|---|---|---|---|
| 6C5 | 1.79E+04 | 4.36E−04 | 2.44E−08 |
| 6A3 | 1.89E+04 | 1.46E−04 | 7.74E−09 |
| 1A11 | — | 2.51E−04 | 3.44E−09 |
| GR34 | — | 8.75E−04 | 15.3E−09 |

### 2.6 Antibody Profile Summary

Antibody 9B7 was found to bind tightly to CD127 with a dissociation constant of 556 μM. It was also capable of partially blocking the binding of IL-7 to CD127, correlating to the partial blocking of IL-7-induced STAT-5 phosphorylation in human CD4 cells (FIG. 4).

Antibody 6C5 (mouse IgG1) was determined to inhibit pSTAT5 signalling with an IC₅₀ of 50 μg/ml.

Antibody 6A3 (mouse IgG1) was determined to inhibit pSTAT5 signalling with an IC₅₀ of 0.099 μg/ml in the assay described herein. It had an affinity for the IL-7Rα EDC of 7.99 nM (KD) and a Kd of 3.34×10⁻⁴. It was capable of binding to IL-7Rα expressed on CHO with an EC₅₀ of 0.19 μg/ml, and blocked IL-7/IL-7Rα with an IC₅₀ of 1.92 μg/ml. 6A3 was determined to bind to amino acids within CD127 epitope regions 2, 3, 4 and 5 (SEQ ID NOs:118-121).

Antibody 1A11 (mouse IgG1) was determined to inhibit pSTAT5 signalling with an IC₅₀ of 0.088 μg/ml in the assay described herein. It had an affinity for the IL-7Rα EDC of 3.44 nM (KD) and a Kd of 2.51×10⁻⁴. It was capable of binding to IL-7Rα expressed on CHO with an EC₅₀ of 0.16 μg/ml, and blocked IL-7/IL-7Rα with an IC₅₀ of 1.79 μg/ml. 1A11 was determined to bind to amino acids within CD127 epitope regions 2, 3, 4 and 5 (SEQ ID NOs:118-121).

Antibody GR34 (mouse IgG1) was determined to inhibit pSTAT5 signalling with an IC₅₀ of 0.22 μg/ml in the assay described herein. It had an affinity for the IL-7Rα EDC of 15.3 nM (KD) and a Kd of 8.75×10⁻⁴. It was capable of binding to IL-7Rα expressed on CHO with an EC₅₀ of 0.27 μg/ml, and blocked IL-7/IL-7Ra with an IC₅₀ of 2.29 μg/ml. GR34 was determined to bind to amino acids within CD127 epitope regions 2, 3, 4 and 5 (SEQ ID NOs:118-121).

Commercial antibody R.3434 (Dendritics) was determined to inhibit pSTAT5 signalling with an IC₅₀ of 0.67 μg/ml in the assay described herein. It had an affinity for the IL-7Rα EDC of 7.74 nM (KD) and a Kd of 1.46×10⁻⁴. It was capable of binding to IL-7Rα expressed on CHO with an EC₅₀ of 0.01 μg/ml, and blocked IL-7/IL-7Rα with an IC₅₀ of 1.38 μg/ml. R.3434 was determined to bind to amino acids within CD127 epitope regions 2, 3, 4 and 5 (SEQ ID NOs:118-121).

### 2.7 Sequencing of Variable Domains

### 2.7.1 9B7

Total RNA was extracted from pellets of 2×10⁷ 9B7 clone cells using the Oligotex Direct mRNA Kit from Qiagen according to the manufacturer's instruction. The reverse transcription of mRNA to cDNA was performed with ImProm-II™ Reverse Transcription System (Promega) according to the manufacturer's instruction with conventional primers for mouse VH and VK genes. 7 reactions for heavy chain variable region and 6 reactions for light chain variable region were amplified.

The purified RT-PCR fragments were cloned into pMD18-T vector (Takara) and a consensus sequence was obtained for each hybridoma by sequence alignment, database searching and alignment with known immunoglobulin variable sequences listed in KABAT (Kabat, E. A., Wu, T. T., Perry, H. H., Gottesman, K. S., Foeller, C., 1991. Sequences of proteins of Immunological Interest, 5^{th} edition, US Department of Health and Human Services, Public Health Service, NIH).

### The Consensus Sequence of mAb 9B7 was:

Rearranged VH of mAb 9B7 used a V segment of the
Igh-VQ52 VH2 family.
(SEQ ID NO. 2)
QVQLQESGPGLVAPSQSLSITCTVSGFSLSRYNVHWVRQPPGKGLEWLGM
IWDGGSTDYNSALKSRLSITKDNSKSQVFLKMNSLQTDDTAMYYCARNRY
ESGMDYWGQGTTVTVSS
FR1 sequence:
(SEQ ID NO: 12)
QVQLQESGPGLVAPSQSLSITCTVSGFSLS
CDR1 sequence:
(SEQ ID NO: 4)
RYNVH
FR2 sequence:
(SEQ ID NO: 13)
WVRQPPGKGLEWLG
CDR2 sequence:
(SEQ ID NO: 5)
MIWDGGSTDYNSALKS
FR3 sequence:
(SEQ ID NO: 14)
RLSITKDNSKSQVFLKMNSLQTDDTAMYYCAR
CDR3 sequence:
(SEQ ID NO: 6)
NRYESG
FR4 sequence:
(SEQ ID NO: 15)
MDYVVGQGTTVIVSS
Rearranged Vk of mAb 9B7 used a V segment of the
IGKV8 family
(SEQ ID NO: 3)
DIVMTQTPSSLTVTAGEKVTMSCKSSQSLLNSGNRKNYLTWYQQKPGQSP
KWYWASTRESGVPDRFTGSGSGTDFTLIISSVQAEDLAVYYCQNDYTYPF
TFGSGTKLEIKR
FR1 sequence:
(SEQ ID NO: 16)
DIVMTQTPSSLTVTAGEKVTMSC
CDR1 sequence:
(SEQ ID NO: 7)
KSSQSLLNSGNRKNYLT
FR2 sequence:
(SEQ ID NO: 17)
WYQQKPGQSPKLLIY
CDR2 sequence:
(SEQ ID NO: 8)
WASTRES
FR3 sequence:
(SEQ ID NO: 18)
GVPDRFTGSGSGTDFTLIISSVQAEDLAVYYC
CDR3 sequence:
(SEQ ID NO: 9)
QNDYTYPFTFGS
FR 4 sequence:
(SEQ ID NO: 19)
GTKLEIKR
(CDR regions are in bold. Ig gene: Immunoglobulin
gene. VH: Antibody heavy chain variable region.
VL: Antibody light chain variable region. FR:
Framework region. CDR: Complementarity determining
region)

### 2.7.2 6C5

The 6C5 antibody was determined to have the following heavy and light chain variable regions (the CDRs of 6C5, according to Kabat, are shown in bold):

Heavy chain variable region of 6C5
(SEQ ID NO: 29)
EVKLLESGGGLVQPGGSLKLSCAASGFAFSAYWMSWVRQAPGKGLEWIGE
INPDSSTINCTPSLKDKFIISRDNAKNTLSLQMNKVRSEDTALYYCARRL
RPFWYFDVWGAGTTVTVSS
FR1 sequence:
(SEQ ID NO: 37)
EVKLLESGGGLVQPGGSLKLSCAASGFAFS
CRDH1 sequence:
(SEQ ID NO: 31)
AYWMS
FR2 sequence:
(SEQ ID NO: 38)
WVRQAPGKGLEWIG
CDRH2 sequence:
(SEQ ID NO: 32)
EINPDSSTINCTPSLKD
FR3 sequence:
(SEQ ID NO: 39)
KFIISRDNAKNTLSLQMNKVRSEDTALYYCAR
CDRH3 sequence:
(SEQ ID NO: 33)
RLRPFWYFDVW
FR4 sequence:
(SEQ ID NO: 40)
GAGTTVTVSS
Light chain variable region of 6C5
(SEQ ID NO: 30)
DVLMTQTPLSLPVSLGDQASISCRSSQSIVQSNGNTYLEWYLQKPGQSPK
LLIYKVSNRFSGVPDRFSGSGSGTDFTLKISRVEAEDLGVYYCFQGSHVP
RTFGGGTKLEIK
FR1 sequence:
(SEQ ID NO: 41)
DVLMTQTPLSLPVSLGDQASISC
CDRL1 sequence:
(SEQ ID NO: 34)
RSSQSIVQSNGNTYLE
FR2 sequence:
(SEQ ID NO: 42)
WYLQKPGQSPKLLIY
CDRL2 sequence:
(SEQ ID NO: 35)
KVSNRFS
FR3 sequence:
(SEQ ID NO: 43)
GVPDRFSGSGSGTDFTLKISRVEAEDLGVYYC
CDRL3 sequence:
(SEQ ID NO: 36)
FQGSHVPRT
FR4 sequence:
(SEQ ID NO: 44)
FGGGTKLEIK

### 2.7.3 6A3

The 6A3 antibody was determined to have the following heavy and light chain variable regions (the CDRs of 6A3, according to Kabat, are shown in bold):

Heavy chain variable region of 6A3
(SEQ ID NO: 51)
DVQLQESGPGLVKPSQSLSLTCTVTGYSITTDYAWNWIRQFPGNKLEWMG
YIFYSGSTTYTPSLKSRISITRDTSKNQFFLQLNSVTTEDTATYYCARGG
YDVNYFDYWGQGTTLTVSS
FR1 sequence:
(SEQ ID NO: 59)
DVQLQESGPGLVKPSQSLSLTCTVTGYSIT
CRDH1 sequence:
(SEQ ID NO: 53)
TDYAWN
FR2 sequence:
(SEQ ID NO: 60)
WIRQFPGNKLEWMG
CDRH2 sequence:
(SEQ ID NO: 54)
YIFYSGSTTYTPSLKS
FR3 sequence:
(SEQ ID NO: 61)
RISITRDTSKNQFFLQLNSVTTEDTATYYCAR
DRH3 sequence:
(SEQ ID NO: 55)
GGYDVNYF
FR4 sequence:
(SEQ ID NO: 62)
DYWGQGTTLTVSS
Light chain variable region of 6A3
(SEQ ID NO: 52)
DIQMTQSPASQSASLGESVTITCLASQTIGAWLAWYQQKPGKSPQLLIYA
ATRLADGVPSRFSGSGSGTKFSFKISSLQAEDFVSYYCQQFFSTPWTFGG
GTKLEIK
FR1 sequence:
(SEQ ID NO: 63)
DIQMTQSPASQSASLGESVTITC
CDRL1 sequence:
(SEQ ID NO: 56)
LASQTIGAWLA
FR2 sequence:
(SEQ ID NO: 64)
WYQQKPGKSPQLLIY
CDRL2 sequence:
(SEQ ID NO: 57)
AATRLAD
FR3 sequence:
(SEQ ID NO: 65)
GVPSRFSGSGSGTKFSFKISSLQAEDFVSYYC
CDRL3 sequence:
(SEQ ID NO: 58)
QQFFSTPWT
FR4 sequence:
(SEQ ID NO: 66)
FGGGTKLEIK

### 2.7.4 1A11

The 1A11 antibody was determined to have the following heavy and light chain variable regions (the CDRs of 1A11, according to Kabat, are shown in bold):

VH of mAb 1A11
(SEQ ID NO: 71)
EVQLQQSGPELLKPGASMKISCKASGYSFTGYTMNWVKQSHGKNLEWIGL
INPYNGVTSYNQKFKGKATLIVAKSSSTAYMELLSLTSEDSAVYYCARGD
GNYWYFDVWGAGTTVTVSS
FR1 sequence:
(SEQ ID NO: 79)
EVQLQQSGPELLKPGASMKISCKASGYSFT
CDRH1 sequence:
(SEQ ID NO: 73)
GYTMN
FR2 sequence:
(SEQ ID NO: 80)
WVKQSHGKNLEWIG
CDRH2 sequence:
(SEQ ID NO: 74)
LINPYNGVTSYNQKFK
FR3 sequence:
(SEQ ID NO: 81)
GKATLTVAKSSSTAYMELLSLTSEDSAVYYCAR
CDRH3 sequence:
(SEQ ID NO: 75)
GDGNYWYF
FR4 sequence:
(SEQ ID NO: 82)
DVWGAGTTVTVSS
Vk of mAb 1A11
(SEQ ID NO: 72)
EIVLTQSPAITAASLGQKVTITCSASSSVTYMHWYQQKSGTSPKPWIYEI
SKLASGVPVRFSGSGSGTSYSLTISSMEAEDAAIYYCQEWNYPYTFGGGT
KLEIK
FR1 sequence:
(SEQ ID NO: 83)
EIVLTQSPAITAASLGQKVTITC
CDRL1 sequence:
(SEQ ID NO: 76)
SASSSVTYMHW
FR2 sequence:
(SEQ ID NO: 84)
YQQKSGTSPKPWIY
CDRL2 sequence:
(SEQ ID NO: 77)
EISKLAS
FR3 sequence:
(SEQ ID NO: 85)
GVPVRFSGSGSGTSYSLTISSMEAEDAAIYYC
CDRL3 sequence:
(SEQ ID NO: 78)
QEWNYPYTF
FR4 sequence:
(SEQ ID NO: 86)
GGGTKLEIK

### 2.7.5 R3434

R3434 is commercially available from Dendritics, Inc. In-house sequence analysis of in-gel digested protein involved N-terminal sequence analysis using Edman degradation on an ABI Procise 494 automated protein sequencer (Applied Biosystems, Foster City, Calif., USA), peptide mass fingerprinting and MALDI-LIFT-MS/MS sequencing on a Bruker Ultraflex III Maldi-TOF mass spectrometer and additional LC-ESI-MS/MS sequencing on a Bruker HCT+ ion-trap mass spectrometer (both from Bruker Daltonics, Bremen, Germany). The reverse engineered clone was named GR34, the sequence of which is below.

Rearranged VH of mAb GR34
(SEQ ID NO: 90)
EVQLQQSGPELVKPGASMKISCKASGYSFTGYTMNWVKQSHGKNLEWIGL
INPYSGITSYNQNFKGKATLTVDKSSSTAYMELLNLTSEDSAVYYCARGD
GNYWYFDVWGAGTTVTVSS
FR1 sequence:
(SEQ ID NO: 98)
EVQLQQSGPELVKPGASMKISCKASGYSFT
CDR1 sequence:
(SEQ ID NO: 92)
GYTMN
FR2 sequence:
(SEQ ID NO: 99)
WVKQSHGKNLEWIG
CDR2 sequence:
(SEQ ID NO: 93)
LINPYSGITSYNQNFK
FR3 sequence:
(SEQ ID NO: 100)
GKATLTVDKSSSTAYMELLNLTSEDSAVYYCAR
CDR3 sequence:
(SEQ ID NO: 94)
GDGNYWYF
FR4 sequence:
(SEQ ID NO: 101)
DVWGAGTTVTVSS
Rearranged Vk of mAb GR34
(SEQ ID NO: 91)
EIILTQSPAITAASLGQKVTITCSASSSVSYMHWYQQKSGTSPKPWIYEI
SKLASGVPARFSGSGSGTSYSLTISSMEAEDAAIYYCQYWNYPYTFGGGT
KLEIK
FR1 sequence:
(SEQ ID NO: 102)
EIILTQSPAITAASLGQKVTITC
CDR1 sequence:
(SEQ ID NO: 95)
SASSSVSYMHW
FR2 sequence:
(SEQ ID NO: 103)
YQQKSGTSPKPWIY
CDR2 sequence:
(SEQ ID NO: 96)
EISKLAS
FR3 sequence:
(SEQ ID NO: 104)
GVPARFSGSGSGTSYSLTISSMEAEDAAIYYC
CDR3 sequence:
(SEQ ID NO: 97)
QYWNYPYTF
FR4 sequence:
(SEQ ID NO: 105)
GGGTKLEIK

### 2.8 Identification of 9B7 Epitope by Peptide ELISA

The epitope of the anti-hCD127 antibody 9B7 was determined by peptide ELISA as before (1.2). The results of this mapping are shown in Table 3.

TABLE 4
shows three positive regions of hCD127 identified by
peptide ELISA using clone 9B7

| | | | | |
|---|---|---|---|---|
| Region 1 | 35 | LDDYSFSCYSQLEVN | 49 | SEQ ID NO: 20 |
| Region 2 | 84 | NFRKLQEIYFIETKKFLLIGKS | 105 | SEQ ID NO: 21 |
| Region 3 | 171 | QEKDENKWTH | 180 | SEQ ID NO: 22 |

### 2.9 Determination of Antibody Binding Epitope of 6C5 and R.34.34 by Surface Plasmon Resonance (BIAcore)

15 mers with 7 to 8 overlapping peptides of CD127 ECD were synthesized by Shanghai Science peptide Biology Technology, and by GL Biochem (Shanghai) Ltd. All peptides were prepared by continuous-flow solid phase peptide synthesis. The peptides were then biotinylated at the N-terminus of the peptide with a spacer Acp between the peptide and the biotin moiety, i.e., biotin-Acp-peptide.

The binding of anti-CD127 antibodies to 15 mers synthesized peptide of human CD127 was assessed using a Biacore T100 system (GE Healthcare). Briefly, anti-CD127 antibody was immobilized on a CM5 biosensor chip with a final level of ˜1000 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was activated/deactivated using EDC/NHS/ethanol amine. 1 μM peptides were injected for 120 s at a flow rate of 10 uL/min. Data were analyzed using the Biacore evaluation software package. The runs were carried out at 25° C.

TABLE 5
shows postivie region for 6C5 and R34.34 antibody
identified by BIAcore

| | | | | | |
|---|---|---|---|---|---|
| 6C5 | Region 1 | 65 | NTTNLEFEICGALVE | 79 | SEQ ID NO: 45 |
| R34.34 | Region 1 | 65 | NTTNLEFEICGALVE | 79 | SEQ ID NO: 106 |
| | Region 2 | 80 | VKCLNFRKLQEIYFI | 94 | SEQ ID NO: 107 |
| | Region 3 | 95 | ETKKFLLIGKSNICV | 109 | SEQ ID NO: 108 |
| | Region 4 | 155 | LQKKYVKVLMHDVAY | 169 | SEQ ID NO: 109 |
| | Region 5 | 162 | VLMHDVAYRQEKDEN | 176 | SEQ ID NO: 110 |

### 2.10 Prediction of Epitopes by Biopanninq Phage Peptide Display

To predict the epitope of the anti-hCD127 antibodies, phage display was carried out as before (Section 1.3) on antibodies 9B7, 6C5, R3434, 6A3 and 1A11.

### 2.10.1 9B7

Phage peptide consensus sequence motif or mimotopes identified from the 2 random peptide libraries predicted discontinuous epitopes of mAb 9B7 (Table 4).

TABLE 6
shows three positive regions of hCD127 identified by
phage peptide library using clone 9B7

| | | | | |
|---|---|---|---|---|
| Region 1 | 80 | VKCLNFRKLQEIYFI | 94 | (SEQ ID NO: 23) |
| Region 2 | 95 | ETKKFLLIGKSNICV | 109 | (SEQ ID NO: 24) |
| Region 3 | 170 | RQEKDENKWTHVNLS | 184 | (SEQ ID NO: 25) |

Summary of epitope mapping by phage peptide display and peptide ELISA: three regions were identified as potential epitopes of CD127 for 9B7 monoclonal antibody, as shown below:

| | |
|---|---|
| position 35 LDDYSFSCYSQLEVN 49; | (SEQ ID NO: 26) |
| position 84 NFRKLQEIYFIETKKFLLIGKS 105 | (SEQ ID NO: 27) |
| position 139 YREGANDFVVTFNTSHLQKKYVKVLMHDVAYRQEKDENKWTH 180 | (SEQ ID NO: 28) |

### 2.10.2 6C5

Phage peptide mimotopes identified from the 2 random libraries predicted 2 possible discontinuous epitopes of antibody 6C5.

TABLE 7
shows 6C5 epitope regions identified by phage
peptide library

| | | | | |
|---|---|---|---|---|
| Region 1 | 55 | LTCAFEDPD | 63 | (SEQ ID NO: 46) |
| Region 2 | 209 | PDHYFKGFWSE | 219 | (SEQ ID NO: 47) |

Summary of epitope mapping by phage peptide display and peptide BIAcore: three regions were identified as potential epitopes of CD127 for 6C5, as shown below:

| | |
|---|---|
| Position 55 LTCAFEDPD 63 | (SEQ ID NO: 48) |
| Position 65 NTTNLEFEICGALVE 79 | (SEQ ID NO: 49) |
| Position 209 PDHYFKGFWSEE 219 | (SEQ ID NO: 50) |

### 2.10.3 R.34.34

TABLE 8
shows R34.34 epitope regions identified by phage peptide library

| | |
|---|---|
| Region 1 | 65 NTTNLEFEICGALVEVKCLNFRKLQEIYFIETKKFLLIGK 104 |
| | (SEQ ID NO: 111) |
| Region 2 | 153 SHLQKKYVKVLMH 165 |
| | (SEQ ID NO: 112) |
| Region 3 | 211 HYFK 214 |
| | (SEQ ID NO: 113) |

Summary of epitope mapping by phage peptide display and peptide BIAcore: three regions were identified as potential epitopes of CD127 for R34.34, as shown below:

| | |
|---|---|
| Position 65 NTTNLEFEICGALVEVKCLNFRKLQEIYFIETKKFLLIGK 104 | (SEQ ID NO: 114) |
| Position 153 SHLQKKYVKVLMH 165 | (SEQ ID NO: 115) |
| Position 211 HYFK 214 | (SEQ ID NO: 116) |

### 2.10.4 6A3

Phage peptide consensus sequence motif or mimotopes identified from the 2 random peptide libraries predicted discontinuous epitopes of mAb 6A3.

TABLE 9
shows 6A3 epitope regions identified by phage peptide libraries

| | | |
|---|---|---|
| Region 1 | 66 TTNLEFEICGAL 77 | (SEQ ID NO: 67) |
| Region 2 | 91 IYFIETKKFLLIGKSNICV 109 | (SEQ ID NO: 68) |
| Region 3 | 152 TSHLQKKYVKVL 163 | (SEQ ID NO: 69) |
| Region 3 | 212 YFKGFWSEWSP 222 | (SEQ ID NO: 70) |

It is postulated that these regions may be closely neighbouring regions within an important effector site of CD127, potentially involved in the site of IL-7 binding.

### 2.10.5 1A11

Phage peptide consensus sequence motif or mimotopes identified from the 2 random peptide libraries predicted discontinuous epitopes of mAb 1A11.

TABLE 10
shows 1A11 epitope regions identified by phage peptide libraries

| | | |
|---|---|---|
| Region 1 | 79 EVKCLNFRKLQEIYFIETKKF 99 | (SEQ ID NO: 87) |
| Region 2 | 150 FNTSHLQ 156 | (SEQ ID NO: 88) |
| Region 3 | 207 SIPDHYFKGFWSEW 220 | (SEQ ID NO: 89) |

It is postulated that these regions may be closely neighbouring regions within an important effector site of CD127, potentially involved in the site of IL-7 binding.

### 2.11 Antibody Binding Neutralizing Assay by BIAcore

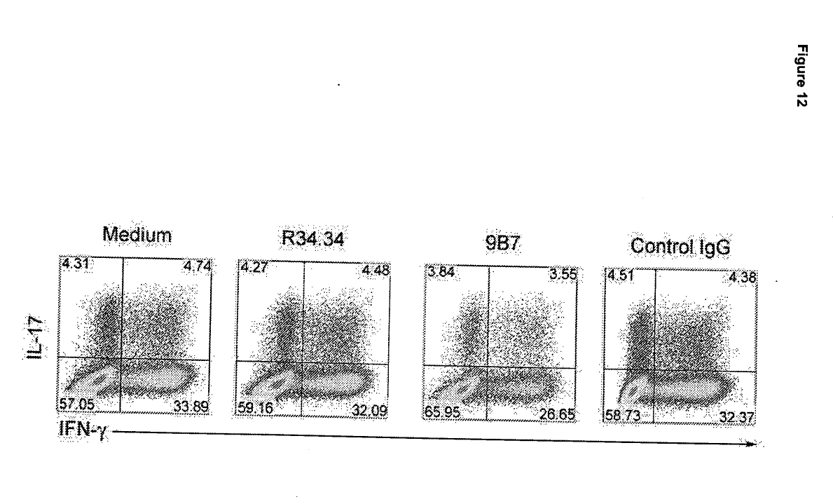

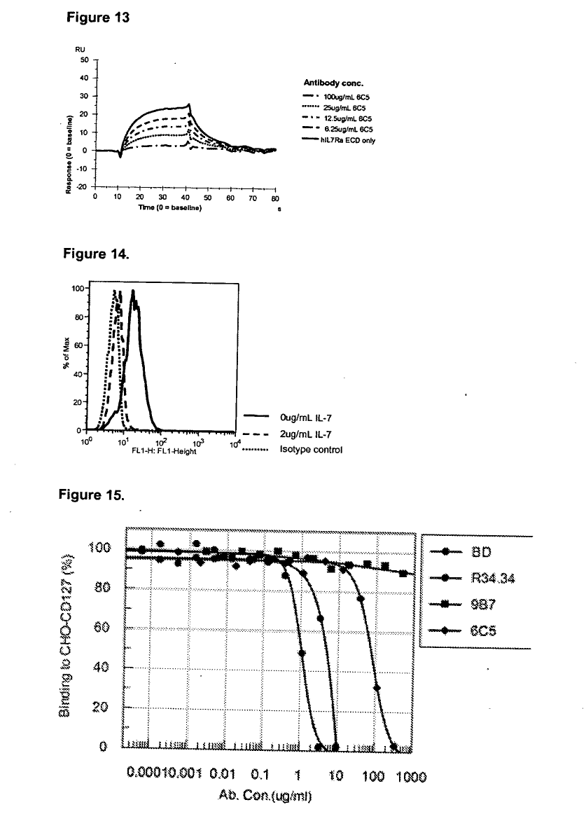

The assay was carried out using a BIAcore T100 system (GE Healthcare) with a reaction temperature of 25° C. Recombinant human IL-7 was immobilized on a CM5 biosensor chip with a final level of ∞500 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was blank immobilized using EDC/NHS/ethanol amine. 10 μg/mL recombinant human CD127 ECD was mixed with various concentrations of anti-CD127 antibodies in separate vials and allowed to incubate for 30 min at 4° C. These mixtures, as well as 10 μg/mL recombinant human CD127 ECD alone, were then injected over the chip surface for 30 s at 10 μL/min. After each injection the sensor chip surfaces were regenerated with 10 mM Glycine-HCl, pH 2.0. At 100 ug/ml antibody 6C5 completely inhibited CD127-ECD binding to IL-7 on the sensor chip. The results for 6C5 are shown in FIG. 13.

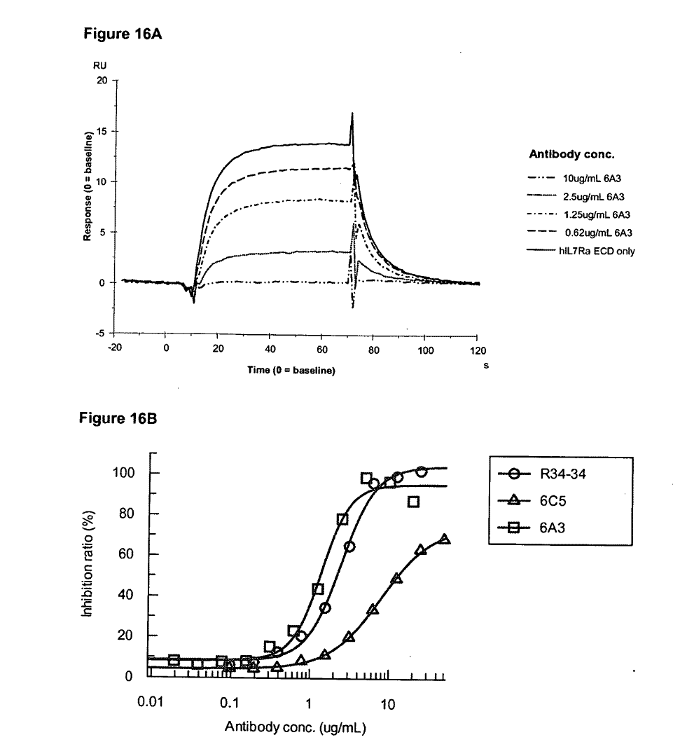

The assay was repeated for 6A3, using a Biacore T100 system (GE Healthcare) with a reaction temperature of 25° C. Recombinant human IL-7 was immobilized on a CM5 biosensor chip with a final level of ˜1000 RU (response units) using the standard amine coupling kit and procedure. HBS-EP buffer pH 7.4 (consisting of 10 mM HEPES, 0.15 M sodium chloride, 3 mM EDTA and 0.005% v/v surfactant P20) was used as running buffer. Sensograms were run against a reference cell that was blank immobilized using EDC/NHS/ethanol amine. 10 μg/mL recombinant human CD127 ECD was mixed with various concentrations of anti-CD127 antibodies in separate vials and allowed to incubate for 1 hr at 4° C. These mixtures, as well as 10 μg/mL recombinant human CD127 ECD alone, were then injected over the chip surface for 60 s at 10 μL/min. After each injection the sensor chip surfaces were regenerated with 10 mM Glycine-HCl, pH 2.0. At 10 μg/ml antibody 6A3 completely inhibited CD127-ECD binding to IL-7 on the sensor chip. The results are shown in FIGS. 16A and 16B. Inhibition ratio calculated as follows: Inhibition ratio=1−RU(sample)/RU(ECD).

### 2.12 IL-7 Competition by FACS

CHO-CD127 cells were prepared and washed by cold Dulbecco's Phosphate-Buffered Saline (DPBS) for 3 times and 2×10⁵ cells were then incubated with 2 μg/mL recombinant IL-7 in separated vials at 4° C. for 30 min. After the incubation, anti-CD127 antibodies were added and the incubation continued for additional 30 min in 4% FCS in DPBS (FACS buffer). Afterward cells were washed in FACS buffer 3 times and stained with anti-mouse IgG ALEXA488 secondary antibody at 1:2000 dilution (Invitrogen Inc. #13-A11017). The cells were then washed 3 times in in FACS buffer and were analyzed in LSR II (BD Biosciences Inc.).

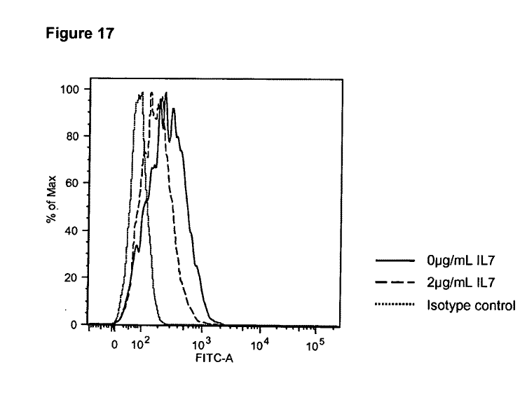

While increasing concentration of IL7, the binding of 6A3, R34.34 or 6C5 to CHO-CD127 is decreased, indicating binding competition of these antibodies with IL-7 to CD127 expressed on CHO cells (FIG. 14 shows the result obtained with 6C5, FIG. 17 shows the result obtained with 6A3). The effect on 9B7 binding was less marked, indicating that 9B7 had less effect on IL-7 competition in this assay.

### 2.13 Antibody Binding Cross-Competition Assay by FACS

CHO-CD127 cells were prepared and washed by cold DPBS for 3 times. Fluorescence labelled anti-CD127 antibody (BD Biosciences Inc #552853) was diluted in FACS buffer and mixed with various concentrations of non-labelled same antibody, or mixed with testing anti-CD127 antibodies, R34.34 and 6C5. The antibody mixtures were then incubated with the CHO-CD127 cells for 30 min at 4° C. After washing in FACS buffer for 3 times, binding of fluorescent labelled BD antibody is measured in LSR II (BD Biosciences Inc.). The results showed that, in addition to unlabeled BD antibody, mAb R34.34 and 6C5 competed for binding with the labelled BD antibody, indicating antibodies BD, R34.34 and 6C5 recognize a similar epitope on CD127 expressed on CHO cells. (FIG. 15).

### EXAMPLE 3

### Treatment Effect of IL-7R Antibody in EAE

The potential for the murine antibodies described in Example 1, to treat MS, was assessed in a mouse EAE model. This experiment has been repeated on multiple occasions; a single representative example is described below.

### Methods

### 3.1 Induction and Evaluation of Experimental Autoimmune Encephalomyelitis (EAE)

Male C57BL/6 mice (6-8 wk; Shanghai Laboratory Animal Center, Chinese Academy of Sciences, Shanghai, China) were immunized s.c. with a synthetic peptide (300 μg) of myelin oligodendrocyte glycoprotein (MOG residues 35-55). Immunization was performed by mixing MOG peptide in complete Freunds adjuvant (CFA, containing 5 mg/ml heat-killed H37Ra strain of Mycobacterium tuberculosis (Difco Laboratories)). Two hundred nanograms of pertussis toxin (List Biological Laboratories) in PBS was administered i.v. on the day of immunization and 48 h later.

For the treatment protocol, a commercially available anti-mouse CD127 mAb was used (BD Bioscience, rat anti-mouse CD127 SB/14, Cat. #550426), a second monoclonal antibody which neutralized IL-7 alone was also tested (R&D systems). The test antibodies or control IgG was administered at 200 μg per mouse i.p. every other day from day 10 onwards till a total of 5 injections. In some experiments, control IgG was replaced by PBS as for the control group. Mice were weighed and examined daily for disease symptoms. They were scored for disease severity using the EAE scoring scale: 0, no clinical signs; 1, limp tail; 2, paraparesis (weakness, incomplete paralysis of 1 or 2 hind limbs); 3, paraplegia (complete paralysis of 2 hind limbs); 4, paraplegia with fore limb weakness or paralysis; 5, moribund state or death.

### 3.2 Histology and Immunohistochemistry

Tissues for histological analysis were removed from mice 21 days after immunization and immediately fixed in 4% paraformaldehyde. Paraffin-embedded 5- to 10-μm sections of spinal cord were stained with Luxol fast blue or H&E and then examined by light microscopy. For immunofluorescence staining of CD4₊ T cells and CD11b+ monocytes/macrophages, spinal cords were removed from mice, perfused with PBS, and incubated in 30% sucrose at 4° C. overnight. The tissue was subsequently dissected and embedded in optimal cutting temperature (OCT) compound. Frozen specimens were sectioned at 7 μm with a cryostat, and the sections mounted upon slides, air dried, and fixed for 10 min with 100% acetone. After blocking with 3% BSA, the sections were incubated overnight with primary rat anti-mouse CD4 or CD11b Abs (BD Biosciences), which were then labeled with Cy3 AffiniPure donkey anti-rat IgG (Jackson ImmunoResearch Laboratories) and examined by immunofluorescence microscopy (Nikon). Isotype-matched Abs were used as negative controls. The degree of demyelination, infiltration of leukocytes, CD4+ T cells and CD11b+ monocytes/macrophages was quantified on an average of 3 spinal cord transverse sections per mouse for a total of 5 mice per group using a previously published procedure.

### 3.3 Proliferation and Cytokine Assays

In proliferation assays, splenocytes (5×10⁵ per well) derived from EAE mice were cultured in triplicate in RPMI 1640 in 96-well plates. Cells were cultured in the presence or absence of the MOG peptide (20 μg/ml) or Con A (2 μg/ml) at 37° C. in 5% CO2 for 72 h. Cells were pulsed with 1 μCi of [3H] thymidine during the last 16-18 h of culture before harvest. [3H] thymidine incorporation in cpm was measured by a MicroBeta counter (PerkinElmer).

For cytokine measurements, supernatants were collected from cell culture at 48 h and diluted for the measurement of IL-1 a, IL-2, IL-4, IL-5, IL-6, IL-17, IFN-γ, IL-23 by using Mouse T_{H}1/T_{H}2 Flowcytomix Multiplex kit and Mouse IL-23 Flowcytomix Simplex kit (Bender MedSystem) according to the manufactures instruction. Briefly, culture supernatants were incubated with the beads mixture coated with capture antibodies and the biotin-conjugated second antibodies mixture at room temperature for 2 hours in dark, PE-labeled streptavidin was added and incubated for 1 hour at room temperature in dark. Data was collected in BD LSR II (Becton Dickinson) and analyzed with BMS FlowCytomix software (Bender MedSystem). Mouse TGF-β and IL-21 were measured by Duoset ELISA kit (R&D Systems) according to the manufacturer's instructions. A standard curve was performed for each plate and used to calculate the absolute concentrations of the indicated cytokines.

### 3.4 Immunoblot Analysis

Protein extracts were loaded onto 10% or 12% SDS-polyacrylamide gels and subjected to electrophoresis. Immunoblot analysis was performed by initial transfer of proteins onto Immobilon-P membrane (Millipore) using a Mini Trans-Blot apparatus (Bio-Rad). After 2 h of blocking, the membranes were incubated overnight at 4° C. with specific primary Abs against P-JAK1, JAK1, P-AKT, AKT, P-Stat3, Stat3, P-Stat5, Stat5, Bcl-2, Bcl-xL, Bim, Bad, P-Bad (All the aforementioned antibodies are from Cell Signal), MCL-1 (Bio-legend), Bax (BD Bioscience), RORγt (Abcam), Foxp3 (Santa Cruz Biotechnology), Actin (Santa Cruz Biotechnology) respectively. After washing and subsequent incubation with a goat anti-rabbit (Sigma-Aldrich) or goat anti-rat Ab (Jackson ImmunoResearch) conjugated with HRP for 1 h at room temperature and extensive washing, signals were visualized with ECL substrate (Pierce).

3.5 cDNA Array Analysis

The expression profile of selected genes related to apoptosis and JAK-STAT signaling pathway was analyzed by using a validated cDNA array system (GEArray S Series, SuperArray Bioscience detailed gene list can be found at the manufacturer's website: www.superarray.com/gene array product/HTML/MM-602.3.html). Briefly, splenocytes were isolated from naive or day 21 EAE mice treated with anti-CD127 mAb or PBS. CD4+ CD25+ T_{reg} and CD4+CD25-non-T_{reg} cells were obtained by magnetic bead separation (Mitenyi Biotec). Total RNA was extracted using Trizol Reagent (Invitrogen). Three micrograms of total RNA were reverse transcribed into biotin-16-deoxy-UTP-labeled single-strand cDNA using an AmpLabeling-LPR Kit (SuperArray). After prehybridization, membranes were hybridized with biotin-labeled sample cDNA and incubated with alkaline phosphatase-conjugated streptavidin (Chemiluminescent Detection kit; SuperArray) to visualize the signal. The results were analyzed using the GEArray Expression Analysis Suite (SuperArray). The results are representative of three experiments using independent splenocyte preparations.

### 3.6 Apoptosis Analysis

Analysis for apoptosis was performed using an annexin V-FITC apoptosis detection kit (BD Biosciences), splenocytes derived from EAE mice were washed and incubated with 5 μl of annexin V-FITC and 5 μl of 7-AAD for 15 min at room temperature. Stained cells were analyzed subsequently using a FACS LSRII instrument (BD) within 1 h.

3.7 Isolation of Mononuclear Cells from Mouse CNS Tissue

Mononuclear cells were prepared from brain and spinal cord using gradient centrifugation. In brief, mice were perfused with 30 ml PBS to remove blood from internal organs. The dissociated brain and spinal cord tissue were grinded and filtered through a 70 μm cell strainer. Resulting cell solution was centrifuged in a Percoll gradient. Mononuclear cells at the interface between two gradients (37% and 70% Percoll, Pharmaica) were collected, washed by centrifugation with medium, and then submitted to FACS analysis.

3.8 Isolation of CD4+ T cells

Spleens of naive mice were removed and dispersed into single cell suspensions. For purification of naïve T cells, CD4⁺ T cells were first purified using CD4 microbeads (Miltenyi) from spleen and lymph nodes of naïve mice. The resulting cells were labeled subsequently with CD44, CD62L and CD25 antibodies and further purified for the CD44^{lo}CD62L^{hi}CD25 population by FACS sorting (FACSAria II, Becton Dickinson). To get CD4⁺CD25^{hi} and CD4⁺CD25⁻ T cells, single cell suspensions were incubated with FITC-labelled anti-CD4 antibody and PE-labeled anti-CD25 antibody (BD Biosciences) on ice for 30 min. CD4⁺CD25^{hi} and CD4⁺CD25⁻ T cells were sorted by a FACSAria instrument (Becton Dickinson). Similar approaches were employed to isolate human CD4⁺CD25⁺ and CD4⁺CD25⁻ T cells. CD4⁺ T cells were first purified from PBMs using a CD4⁺ No-touch T cell isolation kit (Miltenyi Biotec), and CD4⁺CD25⁻ T cells were isolated by negative selection using anti-CD25 microbeads (Miltenyi Biotec). The purity of CD4⁺, CD4⁺CD25⁺, and CD4⁺CD25⁻ T cell fractions was always greater than 95%.

### 3.9 Induction of T_{H}17, T_{H}1 and T_{reg}

Naive mouse CD4+ T cells were plated in 96-well flat-bottomed plates (Costar) at a density of 1×10⁶ cells/ml. Cells were stimulated with plate-bound anti-CD3 Ab (5 μg/ml; BD Bioscience) and anti-CD28 Ab (5 μg/ml; BD Bioscience) in complete medium.

T cells were cultured in T_{H}1 conditions {recombinant IL-12 (10 ng/ml; eBioscience) lus anti-IL-4 (10 μg/ml; BD Bioscience)}, or T_{H}17 conditions {TGF-β1 (1 ng/ml; R&D Systems), IL-23 (10 ng/ml; R&D Systems) and IL-6 (10 ng/ml; eBioscience) plus anti-IFNγ (10 μg/ml; BD Bioscience) and anti-IL-4(10 μg/ml)} for 4 days.

To induce/convert CD4⁺CD25⁺ T_{reg} from CD4⁺CD25⁻ T cells, purified human or mouse CD4⁺CD25⁻ T cells were cultured at 2×10⁶ cells/ml with TGF-β1 (10 ng/ml) and IL-2 (50 lU/ml, R&D Systems) in the presence of coated anti-CD3 antibody (5 μg/ml) and 5 μg/ml anti-CD28 antibody for 4 days. In some cases, medium was washed out from the aforementioned culture systems and cells were then cultured in fresh medium for 1 h or 48 h in the presence or absence of IL-7 (10 ng/ml). To differentiate human T_{H}17 cells, total human CD4+ cells were stimulated in anti-CD3 and anti-CD28 in the presence of IL-1β, IL-6, and IL-23 for six days. IL-7, IL-2, and antibodies were added on day 3 to the differentiation system.

### 3.10 Flow Cytometric Analysis

For surface staining of CD4, CD25, CD8, B220 and CD127, cells were resuspended in PBS containing 1% BSA (Sigma-Aldrich) and 0.1% sodium azide and incubated with fluorochrome-conjugated antibodies to the indicated cell surface markers (BD Bioscience or eBioscience) for 30 minutes on ice. For intracellular cytokine staining, freshly isolated mononuclear cells from lymph nodes, spleens and CNS of EAE mice or in vitro cultured cells were re-stimulated for 5 h with PMA (20 ng/ml) and lonomycin (1 μM) in the presence of GolgiPlug (1:1000 diluted; BD Bioscience). The cells were surface stained with fluorescently labeled antibodies, resuspended in Fixation/Permeabilization solution (BD Bioscience), and stained for intracellular cytokines according to the manufacturer's instructions. Particularly, for IL-7 intracellular staining, cells were firstly incubated with antibodies against mouse CD16/CD32 (BD Bioscience) for 30 min at 4° C. and followed by fixation/permeabilization using BD Bioscience solution, cells were then stained with goat anti-mouse IL-7 IgG (R&D Systems) or goat IgG (R&D Systems) as primary antibodies and Alexa Fluor® 488 donkey anti-goat IgG (Jackson Immunol) as a secondary antibody. Bcl-2 intracellular staining was performed with the same protocol but without the PMA and lonomycin stimulation. As for intracellular staining of Foxp3, cells were fixed and permeabilized with Foxp3 staining buffer (eBioscience). Permeabilized cells were stained with PE or FITC-conjugated anti-human or anti-mouse Foxp3 mAbs (0.5 μg/10⁶ cells; eBioscience). For intracellular staining of phosphorylated yes, cells were fixed for 10 min at 37° C. with 2% (wt/vol) paraformaldehyde, made permeable for 30 min on ice with 90% (vol/vol) methanol, and stained for anti-phosphorylated Stat5 (BD Bioscience) staining. Flow cytometric analysis was performed on BD LSR II (Becton Dickinson) instruments and results were analyzed using FlowJo software (Tree Star Inc.).

### 3.11 Statistical Analysis

Differences in the expression of genes between the groups were analyzed by the Mann-Whitney U test. Two-tailed Student's t test was used to analyze the differences between the groups. One-way ANOVA was initially performed to determine whether an overall statistically significant change existed before using the 2-tailed paired or unpaired Student's t test. A P value of less than 0.05 was considered statistically significant.

### Results

### 3.12 Amelioration of EAE by IL-7R or IL-7 Antagonism

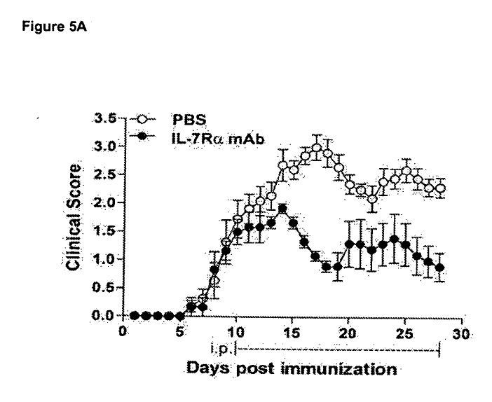

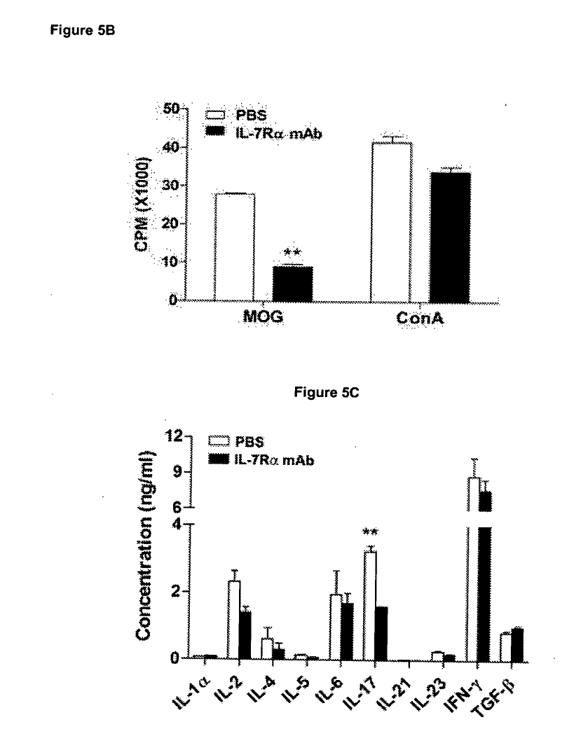

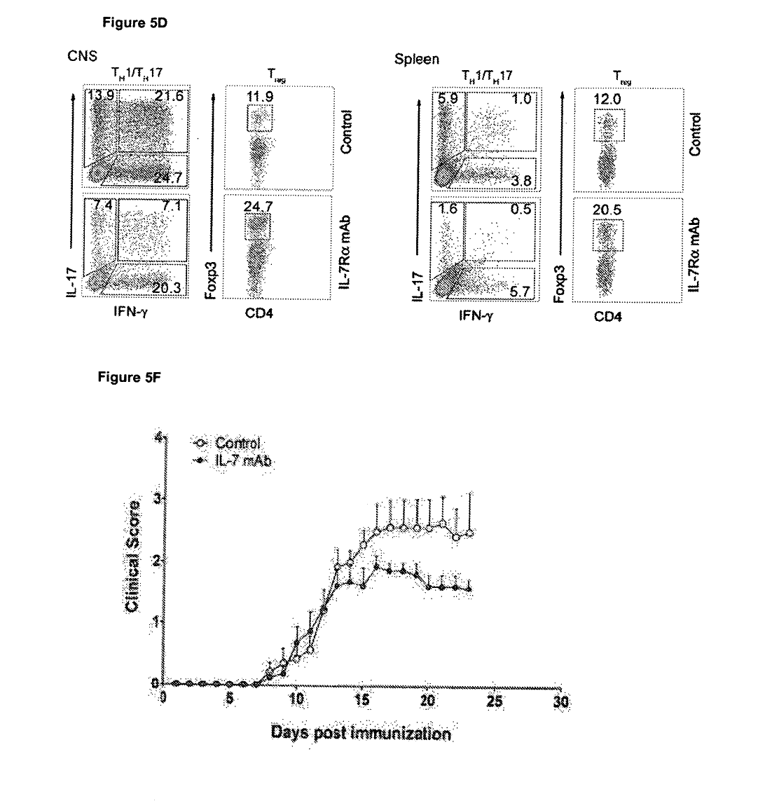

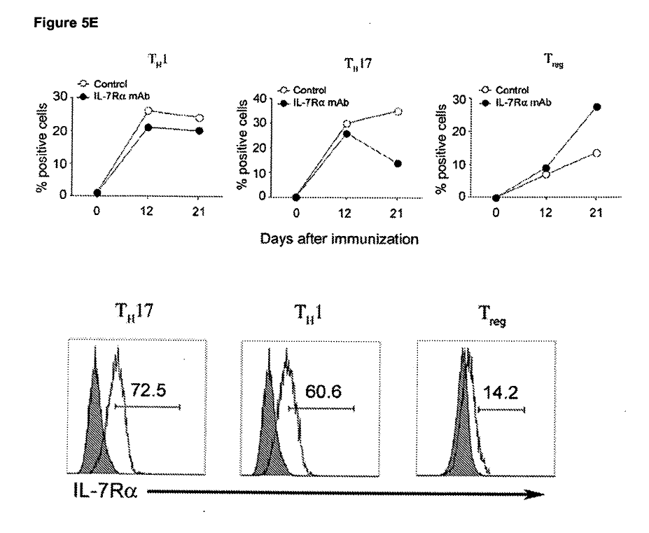

As shown in FIG. 5, when administered three times from Day 10 onwards, anti-CD127 antibody treatment markedly altered the clinical course of EAE by reducing the disease severity compared to an isotype control (FIG. 5A). The treatment regimen resulted in a marked reduction in disease severity accompanied by decreased inflammation and demyelination in affected spinal cord compared to that of control mice. Splenocytes obtained from treated mice exhibited significantly decreased T cell reactivity to MOG but not non-specific T cell activation induced by ConA (FIG. 5B). Noticeably, the treatment effect correlated with a selective reduction in the production of IL-17 among other inflammation-related cytokines in MOG-reactive T cells (FIG. 5C), and in percentage of T_{H}17 cells and to a lesser extent, T_{H}1 cells in both spleen and spinal cord of treated EAE mice (FIG. 5D). The absolute numbers of CNS-infiltrating T_{H}17 cells were decreased by 10-fold in treated mice compared to those of control mice. In contrast, T_{reg} cells increased reciprocally over the course of EAE (FIG. 5D). There was differential expression of IL-7R in the three subsets (FIG. 5E).

It was further evident that T_{H}17 and T_{H}1 cells seen after onset of EAE (day 12 or day 21 post-immunisation) were exclusively the CD44⁺CD62L⁻ memory phenotype and susceptible to IL-7R antibody treatment (data not shown). Although CD4⁺ T cell infiltration in spinal cord was markedly reduced, the absolute number and overall composition of peripheral CD4⁺ and CD8⁺ T cells and B220⁺ B cells were not significantly altered (data not shown). The results indicate that CD4⁺ T cells of the memory phenotype in EAE were highly enriched for pathogenic T_{H}17 and T_{H}1 subsets and susceptible to IL-7R antagonism, which tilts the T_{H}17/T_{H}1 to T_{reg} ratio towards a new balance in treated EAE mice.

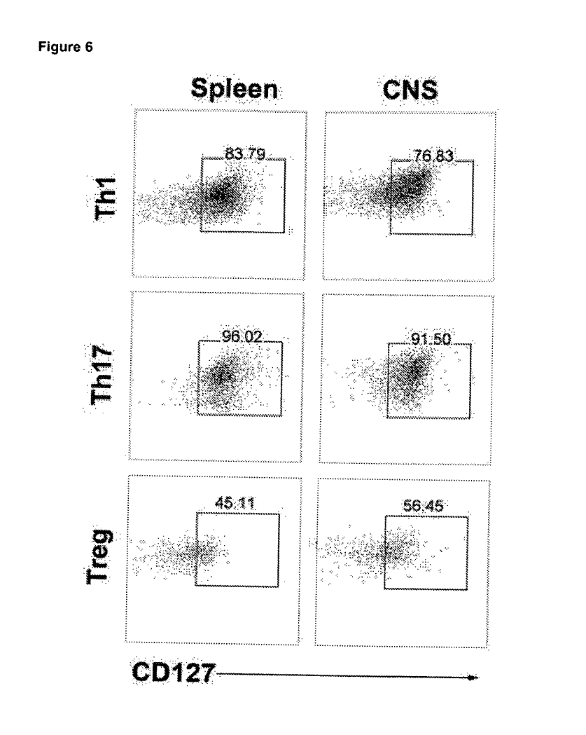

An antibody against IL-7 also attenuated the EAE clinical scores (FIG. 5F), although not quite to the extent seen with the anti-CD127 antibody. Furthermore, as shown in FIG. 6, CD127 was highly expressed in T_{H}1 and T_{H}17 cells derived ex vivo from spleen or spinal cord of EAE mice while the CD127 expression was significantly lower in Foxp3+ T_{reg}.

### 3.13 The Role of IL-7 in T_{H}17 Differentiation

The in vivo development and function of pathogenic T_{H}17 is a dichotomic process comprised of differentiation and survival and expansion. Pro-inflammatory cytokines such as IL-6, IL-1β and IL-21 are critical to T_{H}17 differentiation and the initiation of autoimmune inflammation in EAE, while survival and expansion of T_{H}17 cells is poorly understood and may involve IL-23.

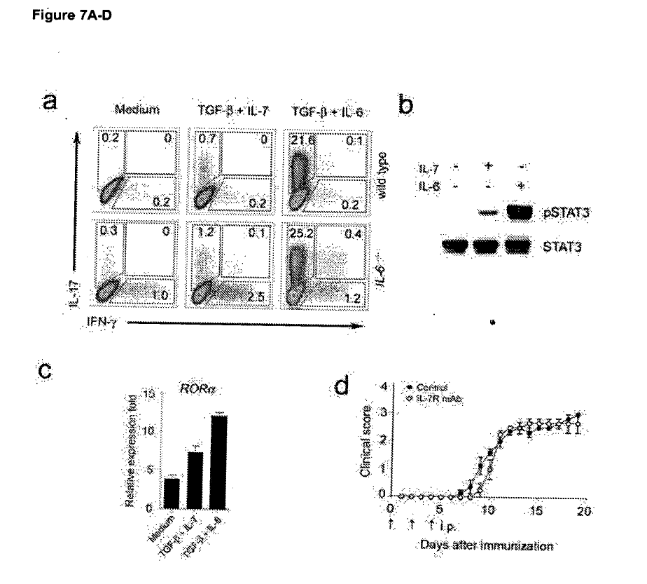

The inventors investigated whether IL-7/IL-7R signalling was associated with T_{H}17 differentiation using purified naive CD4⁺ T cells of the CD44^{lo}CD62L^{hi}CD25⁻ phenotype. The effect of IL-7 was examined by stimulating the resulting cells with CD3/CD28 antibodies in the presence and absence of TGF-β. Although IL-7 promoted T_{H}17 differentiation when combined with TGF-β, the effect was moderate in magnitude compared to that of IL-6 and independent of IL-6 (FIG. 7A), which correlated with marginal induction of STAT-3 phosphorylation and RORα expression by IL-7 (FIG. 7B, FIG. 7C). Similar to IL-6, IL-7 alone did not induce T_{H}17 differentiation (data not shown). Given the moderate effect of IL-7 on T_{H}17 differentiation, we addressed whether the observed effect had in vivo significance in EAE. When administered before onset of EAE (injections at days 0, 2 and 4), the IL-7R antibody treatment did not affect the disease severity even though it slightly delayed the onset as compared to that in mice treated with control antibody (FIG. 7D). The data collectively suggest that IL-7/IL-7R signalling is involved marginally in but not critically required for T_{H}17 differentiation.

### 3.14 Selective Inhibition of T_{H}17 and T_{H}1 Cells in Treated EAE Mice and the Role of CD127 Antagonism in T_{H}17 Development

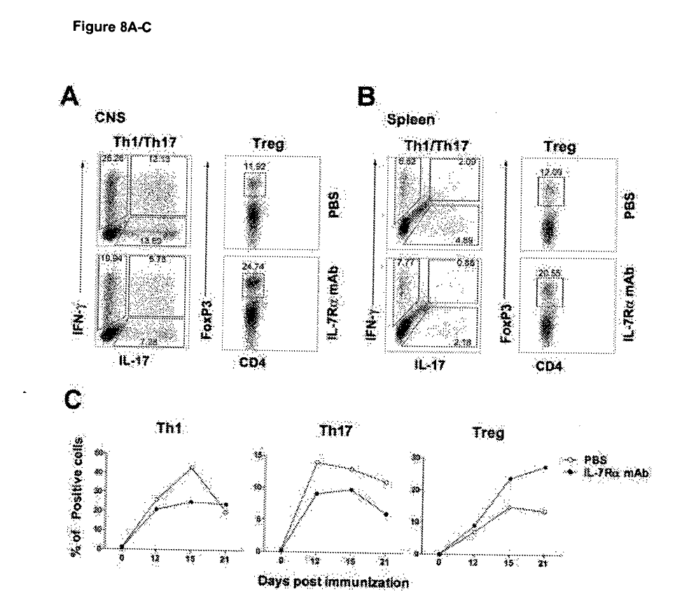

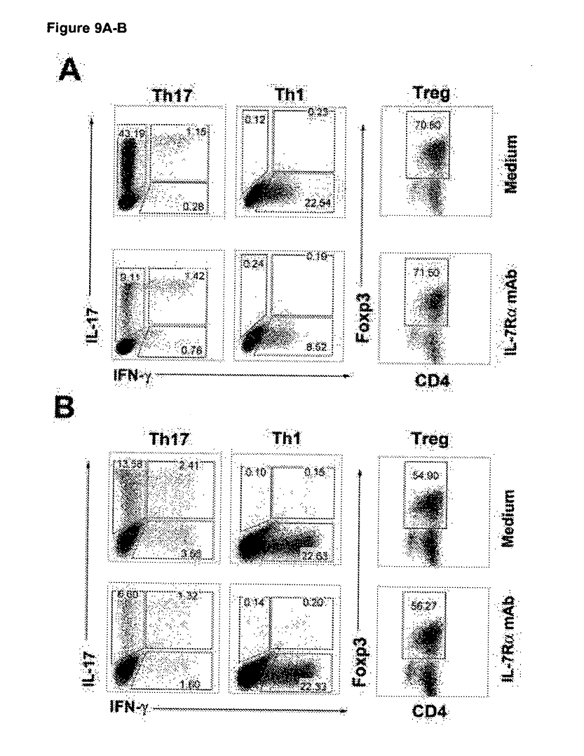

We then examined the role of CD127 antibody in T_{H}17 differentiation and maintenance/expansion in both in vivo and in vitro experimental settings. As shown in FIG. 8A, the percentage of T_{H}17 cells and γ-interferon secreting T_{H}1 cells, to a lesser degree, was decreased in splenocytes and CNS infiltrates in treated EAE mice compared to those of control mice while the levels of Foxp3+ T_{reg} were significantly elevated (FIG. 8B). The changes in the percentage of T_{H}17, T_{H}1 and T_{reg} in the course of EAE in both treated and control mice are presented in FIG. 8C. In a separate in vitro experimental setting, T_{H}17, T_{H}1 and T_{reg} were differentiated, respectively, from naïve splenocytes using different induction protocols in the presence and absence of CD127 antibody.

The result suggested that differentiation of T_{H}17 and, to a lesser extent, T_{H}1 but not T_{reg} was inhibited when CD127 antibody was added in the onset of differentiation (FIG. 9A). A similar effect of CD127 antibody on differentiated T_{H}17 but not T_{H}1 or T_{reg} was seen (FIG. 9B). However, subsequent reruns of this protocol were unable to repeat this initial finding, suggesting that the role of IL-7/1IL-7R signalling is only marginal in differentiation of T_{H}17 cells.

### 3.15 IL-7 Involvement in T_{H}17 Survival and Expansion

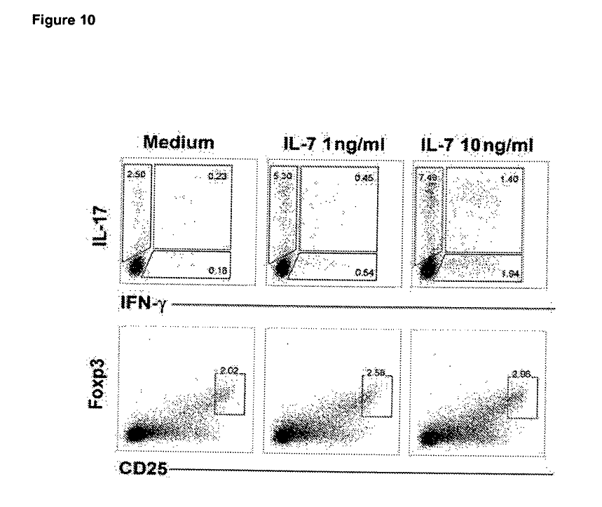

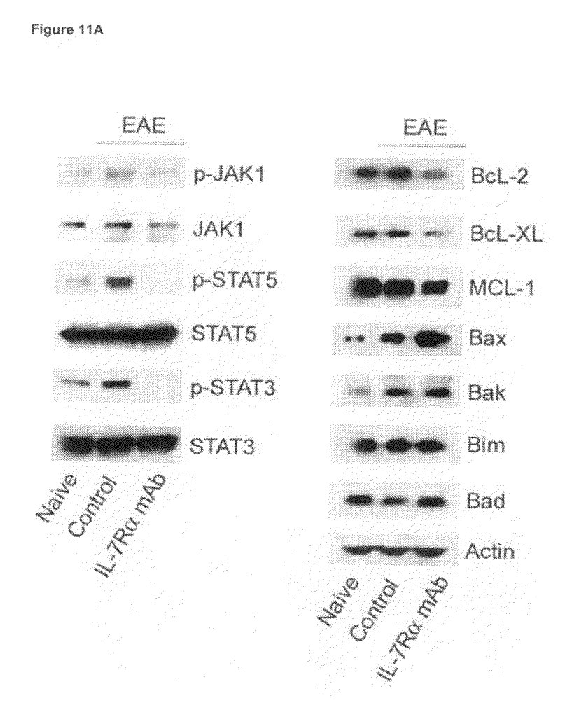

It was of interest to investigate whether IL-7 was required for T_{H}17 differentiation. In this regard, initial results suggested that addition of IL-7 alone was found to increase the differentiation of T_{H}17 and, to a lesser degree, T_{H}1 but not Foxp3 in T_{reg} when day 8 EAE MOG-specific T cells were cultured (FIG. 10).

However, as described herein (Section 3.17), further work revealed the dichotomic process of T_{H}17 development, and suggested that the promotion of T_{H}17 cells was not primarily a result of an increase in differentiation, but a result of an increase in T_{H}17 expansion and survival, in which IL-7 plays a much more significant role.

### 3.16 Susceptibility of T_{H}7 but not T_{reg} to Apoptosis Induced by IL-7R Antagonism

We then investigated the mechanism underlying selective reduction and susceptibility of T_{H}17 by an anti-CD127 antibody. As illustrated in FIG. 11A, immunoblot analysis of CD4+ T cells derived ex vivo from treated or control EAE mice revealed that anti-CD127 antibody treatment resulted in specific changes in signalling pathways related to JAK-STAT and apoptosis as characterized by down-regulation of phosphorylated JAK-1 and phosphorylated STAT-5 and markedly decreased levels of a key pro-apoptotic molecule, BCL-2, and increased activity of an anti-apoptotic molecule, BAX. The modulation of pro- and anti-apoptotic proteins correlated with increased apoptosis level in CD4+ cells in antibody-treated mice. As shown in FIG. 11B, CD127 antibody treatment led to markedly increased percentage of Annexin-V+ apoptotic cells among CD4+CD127+ T cells compared to that of CD4+CD127− T cells derived from treated EAE mice.

It appears that differentiated T_{H}17 cells derived from EAE mice underwent self-initiated or programmed apoptosis that could be reverted by the addition of IL-7. The process was abolished by pre-incubation of susceptible cells with an anti-IL-7R antibody but not a control antibody. IL-7 significantly altered the expression levels of BCL-2, which correlated reciprocally with the levels of Annexin-V⁺ apoptotic cells (FIG. 11C).

The observed effect of IL-7 was clearly mediated through STAT-5 and could be blocked by a STAT-5 specific inhibitor but not by a STAT-3 inhibitor (FIG. 11D) or a P13-K inhibitor (data not shown).

The findings lend further support for the role of IL-7 as a critical survival signal for differentiated T_{H}17 cells to expand by regulating STAT-5 phosphorylation and levels of anti- and pro-apoptotic proteins.

### 3.17 Effect of a Neutralizing Antibody to Human IL-7R on Human T_{H}17 Cells

Our studies in the mouse experimental system showed that T_{H}17 development is a two-step process; “Step 1” being T_{H} precursor cell differentiation, and “Step 2” being T_{H}17 survival/expansion. These two processes are controlled by different cytokines, the expression of which are further regulated by various transcription factors. Both processes contribute critically to the clinical outcome of autoimmune disease. T_{H}17 differentiation is mainly induced by IL-6 through JAK/STAT-3 pathway.

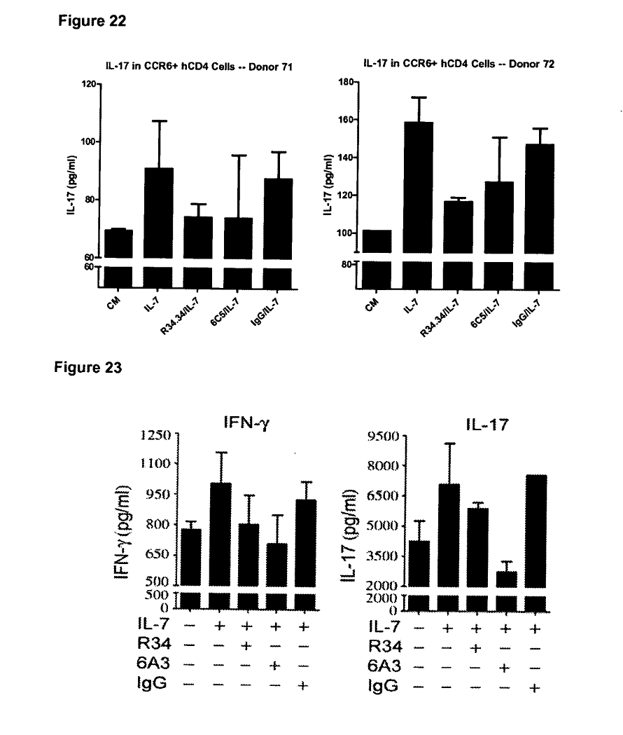

The role of CD127 antagonism in T_{H}17 differentiation was further validated in a human experimental system. When blocking IL-7/IL-7R using an anti-CD127 antibody according to the invention, T_{H}17 differentiation was minimal affected as shown in FIG. 12, indicating that IL-7 plays a minor role in this process. In contrast, our results showed that the major role of IL-7/IL-7R signalling in this two-step cell development process is in Step 2—pathogenic T_{H}17 cell survival and expansion. In this second step, the role of IL-7 is superior to IL-23 through the JAK/STAT-5 pathway. When anti-human IL-7R mAb was given after cells had already committed to T_{H}17 cells, the cells are susceptible to apoptosis as shown in FIG. 22. The study provides compelling evidence for a novel role of IL-7/IL-7R signalling in pathogenic T_{H}17 cell development and functions in EAE and lends strong rationale for IL-7R antagonism as a potential treatment for MS and other autoimmune conditions.

### 3.18 Inhibition of IFNγ Production by IL-7 Stimulated PBMC

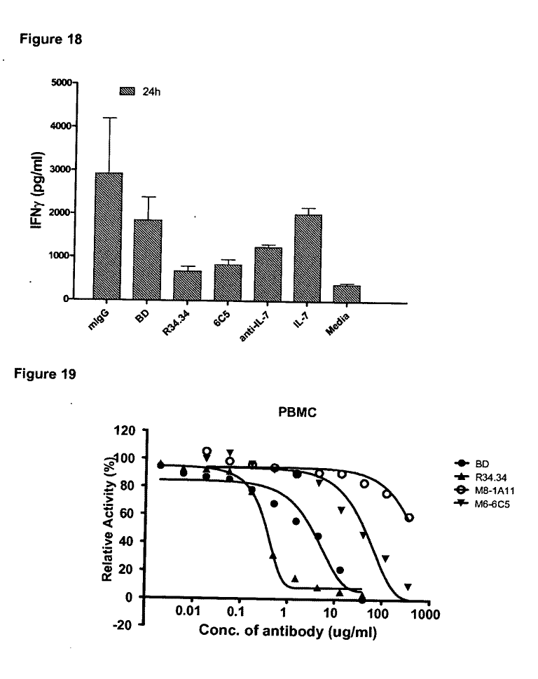

PBMCs were initially screened and selected on the basis of a positive result with antibody R34.34 (Dendritics Inc). Fresh or thawed PBMCs were plated at 2×10⁵ cells/well in 96 well in RPMI 1640 containing 10% FBS. Purified testing antibody 6C5, positive control antibody R34.34 (Dendritics Inc) and anti-human IL-7 (R&D), plus isotype control antibody mouse IgG1 (R&D) were incubated at 10 μg/ml and 100 μg/ml with cells at 37° C. for 30 minutes before 10 ng/ml IL-7 was supplemented. Cells briefly treated with IL-7 served as negative control while non-treated cells as background. 2 μg/ml soluble anti-CD3 and anti-CD28 (eBiosciences) were added to all conditions and the plate was incubated for additional 24 hours at 37° C. with 5% CO₂. IFN-γ level in culture supernatant was analyzed by human IFN-γ ELISA (human IFN-γ ELISA kit, eBiosciences). Under these condition, mAb 6C5 and antibody R34.34 inhibited IL-7 induced IFNγ production (FIG. 18)

### 3.19 Inhibition of IL7 Stimulated IL7-Receptor Signalled Stats Phosphorylation

To screen for antibodies with the ability to block signaling functions of CD127, cryopreserved PBMCs were thawed rapidly and plated in RPMI 1640 medium containing 10% of FBS the night before the functional test. Test sample antibodies and positive control antibody (R34.34, Dendritics Inc #DDX0700; BD anti-CD127, BD Biosciences Inc #552853) were prepared in 3 fold serial dilution starting from a top concentration of 120 ug/ml, and added to 2×10⁵ PBMC cells for 30 mins at 37° C. before stimulation with IL-7 at 1 ng/ml for 15 mins at 37° C. The cells without antibody and IL7 treatment were used as background control. The cells treated with IL7 but not with antibody samples were used as full activity control. Cells after treatment were lysed by lysis buffer (PerkinElmer #TGRS5S500) for 5 mins at 37° C. and the lysates were incubated with Reaction Buffer plus Activation Buffer mix (PerkinElmer #TGRS5S500) containing AlphaScreen® Acceptor beads (PerkinElmer #6760617C) for 2 hours at room temperature. After that, Dilution buffer (PerkinElmer #TGRS5S500) containing AlphaScreen® Donor beads (PerkinElmer #6760617C) were added and incubated for another 2 hours. Luminescence (RFU) from AlphaScreen beads were analyzed on Envision with its default alphascreen mode (top read; Ex 680 nm; Em 570 nm). Results for testing samples were converted to relative activity based on the following formula:

Relative activity (%)=(RFU(sample)−RFU(background control))/(RFU(full activity control)−RFU(background control))

The results of this calculation are shown in FIG. 19.

CCF-CEM cells were cultured in growth medium (RPMI1640, 10% FBS, 100 U/ml Penicillin, 100 ug/ml Streptomycin, 1 mM Sodium Butyrate) and treated with 1 uM Dexamethasone (Sigma #D4902) overnight for IL7 receptor induction before the experiment. Test sample antibodies and positive control antibody (R34.34, Dendritics Inc #DDX0700; BD anti-CD127, BD Biosciences Inc #552853) were prepared in 3 fold serial dilution starting from a top concentration of 120 ug/ml, and added to 2×10⁵ CCF-CEM cells for 30 mins at 37° C. before stimulation with IL-7 at 1 ng/ml for 15 mins at 37° C. The cells without antibody and IL7 treatment were used as background control. The cells treated with IL7 but not with antibody samples were used as full activity control. Cells after treatment were lysed by lysis buffer (PerkinElmer #TGRS5S500) for 5 mins at 37° C. and the lysates were incubated with Reaction Buffer plus Activation Buffer mix (PerkinElmer #TGRS5S500) containing AlphaScreen® Acceptor beads (PerkinElmer #6760617C) for 2 hours at room temperature. After that, Dilution buffer (PerkinElmer #TGRS5S500) containing AlphaScreen® Donor beads (PerkinElmer #6760617C) were added and incubated for another 2 hours. Luminescence (RFU) from AlphaScreen beads were analyzed on Envision with its default alphascreen mode (top read; Ex 680 nm; Em 570 nm). Results for testing samples were converted to relative activity based on the following formula:

Relative activity (%)=(RFU(sample)−RFU(background control))/(RFU(full activity control)−RFU(background control))

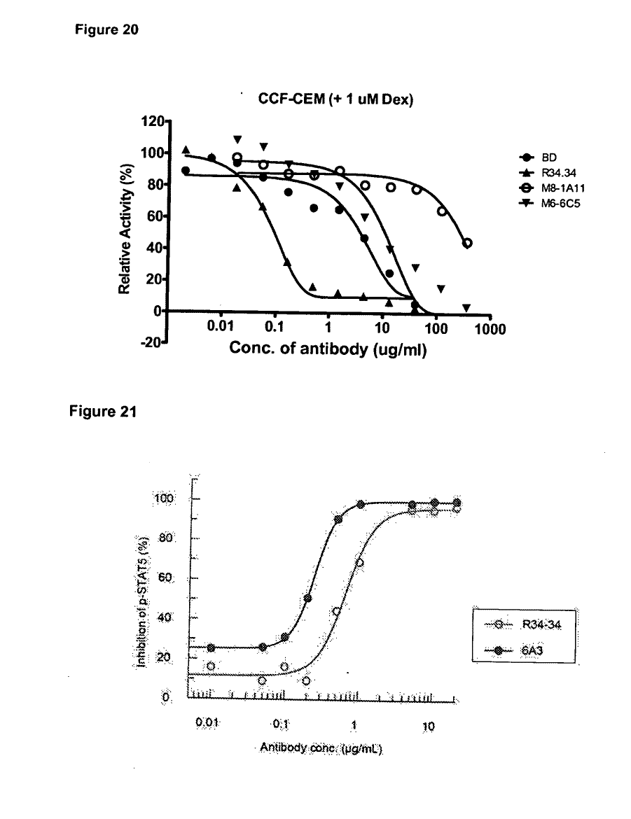

The results are shown in FIG. 20.

The experiment was essentially repeated, for antibody 6A3, as follows. Fresh PBMCs were suspended in serum free RPMI 1640 medium. Test sample antibodies and positive control antibody (6A3 and R34.34, Dendritics Inc #DDX0700) were diluted to achieve final concentrations from 20 ug/ml to 0.01 ug/ml in the culture and were added to 1×10⁶ PBMC cells per sample. PBMCs were incubated with antibodies for 50 mins at 37° C. before stimulation with IL-7 at 1 ng/ml for 15 mins. For intracellular staining of phosphorylated STAT5, cells were fixed for 10 min at 37° C. with 1% (wt/vol) paraformaldehyde, made permeable for 30 min on ice with 90% (vol/vol) methanol, and stained for anti-phosphorylated Stat5 (BD Bioscience) staining. Flow cytometric analysis was performed on BD LSR II (Becton Dickinson) instruments and results were analyzed using FlowJo software (Tree Star Inc.).

The cells without antibody and IL7 treatment were used as background control. The cells treated with IL7 but not with antibody samples were used as full activity control. FIG. 21 shows the inhibition of IL-7-induced P-STAT5 relative to no antibody control at increasing concentrations of R34.34 and 6A3.

### 3.20 Inhibition of IL-7 Induced IL-17 Production in Differentiated T Cells

CD4+cells from six donors were isolated according to the manual (#130-091-155, Miltenyi). Approximately 1×106/ml of the CD4+ cells in 100 ul were mixed with equal volume of 2× T_{H}17 medium (2 μg/ml anti-CD28+10 μg/ml anti-IFNγ+10μg/ml anti-IL-4+12.5 ng/ml IL-1β+20 ng/ml IL-23+50 ng/ml IL-6) and cultured in 37° C. with 5% CO₂ for 5 days. Treatment by the various cytokines and growth factors in the T_{H}17 medium preferentially differentiated the CD4+ cells into T_{H}17 cells. CCR6+ cells from the differentiated cultured cells at day 5 were sorted using BD FACS SORP Aria II. The CCR6+ cells were then adjust to 2×10⁶/ml for the IL-17 production assay.

To measure IL-17 level, 100 μl of CCR6+ cells were pre-incubated with testing antibody for 1 h at 37° C., and then mixed with 100 μl of 20 ng/ml IL-7. The cells were cultured for 3 days in 37° C. with supplement of 5% CO2. IL-17 level in 100 μl of the culture supernatant were measured by FlowCytomix (Bender MedSystems). Table 11 shows the IL-7 and testing antibody (R34.34 and 6C5) concentrations used in generation of the results in FIG. 22 (results from a single donor). R34.34 inhibited IL-17 production in IL-7 induced differentiated T cells in 6/6 donors; 6C5 inhibited IL-17 production in IL-7 induced differentiated T cells in 4/6 donors.

TABLE 11

| | | | | |
|---|---|---|---|---|
| CM | 10 ng/ml IL-7 | 10 μg/ml | 50 μg/ml 6C5 | 50 μg/ml IgG |
| | | R34.34 | 10 ng/ml IL-7 | 10 ng/ml IL-7 |
| | | 10 ng/ml IL-7 | | |

The experiment was essentially repeated for antibody 6A3. CD4+ cells were isolated according to the manual (#130-091-155, Milteni). Approximately 7×10⁵/ml of the CD4+ cells in 100 ul were mixed with equal volume of 2× T_{H}17 medium (2 μg/ml anti-CD28+10 μg/ml anti-IFN-γ+10 μg/ml anti-IL-4+12.5 ng/ml IL-1β+20 ng/ml IL-23+50 ng/ml IL-6) and cultured in 37° C. with 5% CO2 for 5 days. Treatment by the various cytokines and growth factors in the T_{H}17 medium preferentially differentiated the CD4+ cells into Th17 cells. CCR6+ cells from the differentiated cultured cells at day 5 were sorted using BD FACS SORP Aria II. The CCR6+ cells were then adjusted to 2×10⁶/ml for the IL-17 production assay.

To measure IL-17 and IFN-γ level, 100 μl of CCR6+ cells from individual donors were pre-incubated with testing antibody for 1 h at 37° C., and then mixed with 100 μl of 20 ng/ml IL-7. The cells were cultured for 3 days in 37° C. with supplement of 5% CO2. IFN-γ and IL-17 levels in 100 ul of the culture supernatant were measured by FlowCytomix (Bender MedSystems) at 24 h and 40 h, respectively. Table 12 shows the IL-7 and testing antibody concentrations used in generation of the results in FIG. 23. The results are representative of 5/6 donors.

TABLE 12

| | | | | |
|---|---|---|---|---|
| CM | 10 ng/ml IL-7 | 10 μg/ml R34.34 | 10 μg/ml 6A3 | 10 μg/ml IgG |
| | | 10 ng/ml IL-7 | 10 ng/ml IL-7 | 10 ng/ml IL-7 |

### Conclusions

The study described here provides the first immunological evidence supporting the potential role of IL-7 and IL-7R in multiple sclerosis (MS).

The present inventors have provided compelling evidence that IL-7/IL-7R signalling is critically required for survival and expansion of committed T_{H}17 cells in both mouse and human systems, while its role in T_{H}17 differentiation is not essential compared to that of IL-6. IL-7 or IL-7R antagonism administered after EAE onset significantly affect the clinical course of disease. The inventors have therefore shown that IL-7 or IL-7R antagonism provides real therapeutic potential in the treatment of autoimmune diseases and inflammatory disorders in which pathogenic T_{H}17 cells are implicated, particularly MS, and more particularly still the relapsing/remitting course of MS (RRMS).

T_{H}17 development and function is controlled chiefly by IL-6 through JAK/STAT-3 for T_{H}17 differentiation and IL-7 through JAK/STAT-5 for T_{H}17 maintenance. IL-7 not only provides a survival signal for pathogenic T_{H}17 cells but directly induces in vivo T_{H}17 cell expansion, critically contributing to sustained autoimmune pathology in EAE.

As shown in this study, committed T_{H}17 cells of the memory phenotype represent an in vivo pathogenic T cell subset and are susceptible to self-initiated or programmed apoptosis. This process appears to be dependent on IL-7/IL-7R signalling through regulation of pro- and anti-apoptotic proteins, such as Bcl-2 and Bax, in susceptible T_{H}17 cells. In this context, IL-7 serves as a critical survival signal that prevents differentiated T_{H}17 cells from programmed apoptosis. Furthermore, increased IL-7 production and highly expressed IL-7R in pathogenic T cells as seen in the acute phase of autoimmune diseases provide the milieu required for sustained T cell survival and expansion. It is proposed that interaction of IL-7 with its receptor induces aggregation of α and γc chains and activation of down-stream kinases. As a result, the process is likely to alter the cascade of kinase phosphorylation and create a docking site for STAT-5 phosphorylation, which is required for up-regulation of Bcl-2 and Mcl-1 and prevents mitochondria-mediated apoptosis by blocking Bim and Bad from activating Bax and Bak. Thus, it provides an explanation for the involvement of STAT-5 and its association with the anti-apoptotic changes induced in pathogenic T_{H}17 cells by IL-7.

It is surprising that the in vivo effect on the immune system by IL-7R antagonism is highly selective in EAE, affecting T_{H}17 cells and, to a lesser extent, T_{H}1 cells predominantly of the memory phenotype and sparing T_{reg} cells. The inventors have shown that T_{H}17 cell maintenance is affected by IL-7/IL-7R signalling. Under the same experimental conditions, T_{H}1 cells are altered in the in vitro but not in vivo system. The discrepancies may be explained by different cytokine milieu between the in vitro setting where exogenous IL-7 is added and in vivo micro-environment involving interplay of multiple cytokines. The selectivity for T_{H}17 over T_{reg} is readily explained by differential expression of IL-7R, rendering T_{H}17 cells susceptible and T_{reg} cells resistant to IL-7R antagonism. This selectivity appears to play an important role in rebalancing the ratio of pathogenic T_{H}17 cells and T_{reg} cells by IL-7R antagonism in EAE and is attributable to the treatment efficacy. However, the discrepancies in the magnitude of IL-7-induced responsiveness and susceptibility to IL-7R antagonism between T_{H}17 and T_{H}1 could not be simply explained by the expression of IL-7R as both subsets highly express IL-7R. The intrinsic expression and activity of SOCS-1 is responsible for the discrepancies. That is, SOCS-1 naturally expressed in T_{H}1 or experimentally induced in T_{H}17 by IFN-γ is attributable to dampened susceptibility to IL-7 or IL-7R antagonism as SOCS-1 acts as a repressor gene for STAT-5 required for IL-7 signalling. Thus, the selectivity for T_{H}17 cells of the memory phenotype appears to involve intrinsic requirement of these pathogenic cells for IL-7 to survive when activated in the course of EAE. This therapeutic specificity represents an obvious advantage over many other treatment modalities proposed in the autoimmune diseases that often affect a broad spectrum of the immune system/functions.

The novel mechanism of action of IL-7/IL-7R signalling in T_{H}17 cell survival and expansion as discussed above provides powerful explanations for the treatment efficacy of IL-7R antagonism in EAE and therapeutic implications for human autoimmune diseases, such as MS. IL-7 neutralization or IL-7R antagonism is likely to have unique therapeutic advantages. On one hand, the treatment offers the selectivity that distinguishes pathogenic T_{H}1 and T_{H}17 cells from T_{reg} and unrelated immune cells. On the other hand, additional therapeutic advantages of IL-7R antagonism involve its selective effect on survival and expansion of differentiated T_{H}17 as opposed to T_{H}17 differentiation. Targeting, with an inhibitor of the IL-7/IL-7R pathway, the in vivo maintenance of committed T_{H}17 versus T_{H}17 differentiation may be more efficacious in a therapeutic context.

1. A method of treatment of an autoimmune disease or inflammatory disorder in a human subject, comprising administering to the subject an antagonist of at least one of IL-7 receptor mediated T_{H}17 expansion and IL-7 receptor mediated T_{H}17 survival.

2. The method of treatment as claimed in claim 1, wherein the antagonist inhibits IL-7 induced IL-17 production by T_{H}17 cells.

3. The method of treatment as claimed in claim 1 or 2, wherein the antagonist inhibits IL-7 induced IFN-γ production by T_{H}17 cells.

4. The method of treatment as claimed in claim 1, 2 or 3, wherein the antagonist inhibits IL-7 receptor mediated STAT-5 phosphorylation.

5. The method of treatment as claimed in any of claim 1, 2, 3 or 4, wherein the antagonist is a binding protein which specifically binds to IL-7 or to CD127.

6. The method as claimed in claim 5, wherein the binding protein specifically binds to CD127 (SEQ ID NO:1).

7. The method as claimed in claim 6, wherein the binding protein inhibits the binding of IL-7 to IL-7R.

8. The method as claimed in claim 5, 6 or 7, wherein the binding protein binds to at least one amino acid within at least one peptide consisting of amino acid residues selected from the group consisting of:
a) 41 to 63 (SEQ ID NO:117),
b) 65 to 80 (SEQ ID NO:118),
c) 84 to 105 (SEQ ID NO:119),
d) 148 to 169 (SEQ ID NO:120), and
e) 202 to 219 (SEQ ID NO:121),
of SEQ ID NO:1.

9. The method as claimed in claim 8, wherein the binding protein binds to at least one amino acid within each of peptides 65 to 80 (SEQ ID NO:118), 84 to 105 (SEQ ID NO:119), 148 to 169 (SEQ ID NO:120), and 202 to 219 (SEQ ID NO:121), of SEQ ID NO:1.

10. The method of treatment as claimed in claim 1, wherein the binding protein competitively inhibits binding of at least one of:
(i) R34.34 (Dendritics Inc. #DDX0700),
(ii) an antibody having heavy and light variable regions of 6A3 (SEQ ID NO:51 and SEQ ID NO:52, respectively), and
(iii) an antibody having heavy and light chain variable regions of 1A11 (SEQ ID NO:71 and SEQ ID NO:72, respectively),
to human CD127 in an ELISA assay.

11. The method as claimed in any of claims 5 to 10, wherein the binding protein binds to CD127 with an affinity (KD) of 15 nM or less as measured by surface plasmon resonance.

12. The method of treatment as claimed in any preceding claim, wherein the antagonist is an antibody or a fragment thereof.

13. The method as claimed in claim 12, wherein the antibody comprises a heavy chain complementarity determining region 3 (CDRH3) of SEQ ID NO:55 or an analog thereof, or a heavy chain complementarity determining region 3 (CDRH3) of SEQ ID NO:75.

14. The method of treatment as claimed in any preceding claim, wherein the autoimmune or inflammatory disease is associated with elevated levels of IL-17.

15. The method of treatment as claimed in any preceding claim, wherein the human subject has been determined to express an elevated level of IL-17 compared to a healthy human individual.

16. The method as claimed in claim 14 or 15, wherein the antagonist is administered in an amount effective to reduce the level of IL-17 in the patient.

17. The method as claimed in claim 14, 15 or 16, wherein the level of IL-17 is measured in the serum of the patient.

18. The method of treatment as claimed in any preceding claim, wherein the autoimmune disease is multiple sclerosis.

19. The method according to claim 18, wherein the patient has an raised T_{H}17 count within their CD4⁺ T cell population.

20. A method for treating multiple sclerosis in a patient comprising administering an antagonist of IL-7 or CD127 to said patient, wherein the patient is suffering from relapsing remitting multiple sclerosis.

21. A method of treating an autoimmune disease in a human subject, comprising administering to the subject an antagonist of IL-7 or IL-7R in an amount effective to reduce the ratio of T_{H}17 cells relative to T_{H}1 cells.

22. A method of treating an autoimmune disease in a human subject, comprising administering to the subject an antagonist of IL-7 or IL-7R in an amount sufficient to reduce the ratio of T_{H} cells to T_{reg} cells.

23. A method for the treatment of an autoimmune disease in a human subject, comprising administering to the subject an antagonist of IL-7 receptor mediated STAT-5 phosphorylation.

24. The method of treatment as claimed in claim 20, 21, 22 or 23, wherein the antagonist of IL-7 or IL-7R is a binding protein which specifically binds to CD127 or IL-7.

25. The method of treatment as claimed in any of claims 24, wherein the binding protein is an antibody or antigen-binding fragment thereof which binds to at least one amino acid within at least one peptide consisting of amino acid residues:
a) 41 to 63 (SEQ ID NO:117),
b) 65 to 80 (SEQ ID NO:118),
c) 84 to 105 (SEQ ID NO:119),
d) 148 to 169 (SEQ ID NO:120), and
e) 202 to 219 (SEQ ID NO:121),
of SEQ ID NO:1.

26. An isolated human, humanised or chimeric antibody or an antigen-binding fragment thereof, wherein the antibody or fragment thereof binds to an epitope of human CD127 that contains at least one amino acid residue within the region beginning at residue number 80 and ending at residue number 190.

27. The isolated antibody or antibody fragment as claimed in claim 26, wherein the antibody or fragment thereof binds to at least one amino acid within at least one peptide consisting of amino acid residues:
a) 41 to 63 (SEQ ID NO:117),
b) 65 to 80 (SEQ ID NO:118),
c) 84 to 105 (SEQ ID NO:119),
d) 148 to 169 (SEQ ID NO:120), and
e) 202 to 219 (SEQ ID NO:121),
of SEQ ID NO:1.

28. The isolated antibody or antibody fragment as claimed in claim 26 or 27, wherein the antibody or antigen-binding fragment thereof binds to human CD127 with an affinity (KD) which is below 15 nM, as measured by surface plasmon resonance.

29. An isolated binding protein, wherein the binding protein binds to CD127 and comprises a heavy chain complementarity determining region 3 (CDRH3) selected from the group consisting of SEQ ID NO:6, SEQ ID NO:33, SEQ ID NO:55 and SEQ ID NO:75 and analogs thereof.

30. The isolated binding protein as claimed in claim 29, wherein the binding protein comprises:
A: a heavy chain comprising the following CDRs or
analogs thereof

| | |
|---|---|
| CDRH1: RYNVH, | (SEQ ID NO: 4) |
| CDRH2: MIWDGGSTDYNSALKS, | (SEQ ID NO: 5) |
| CDRH3: NRYESG, | (SEQ ID NO: 6) |

and a light chain comprising the following CDRs or
analogs thereof

| | |
|---|---|
| CDRL1: KSSQSLLNSGNRKNYLT, | (SEQ ID NO: 7) |
| CDRL2: WASTRES, | (SEQ ID NO: 8) |
| and | |
| CDRL3: QNDYTYPFTFGS; | (SEQ ID NO: 9) |
| or | |

B: a heavy chain comprising the following CDRs or
analogs thereof

| | |
|---|---|
| CRDH1: AYWMS, | (SEQ ID NO: 31) |
| CDRH2: EINPDSSTINCTPSLKD, | (SEQ ID NO: 32) |
| CDRH3: RLRPFWYFDVW, | (SEQ ID NO: 33) |

and a light chain comprising the following CDRs or
analogs thereof

| | |
|---|---|
| CDRL1: RSSQSIVQSNGNTYLE, | (SEQ ID NO: 34) |
| CDRL2: KVSNRFS, | (SEQ ID NO: 35) |
| and | |
| CDRL3: FQGSHVPRT; | (SEQ ID NO: 36) |
| or | |

C: a heavy chain comprising the following CDRs or
analogs thereof

| | |
|---|---|
| CRDH1: TDYAWN, | (SEQ ID NO: 53) |
| CDRH2: YIFYSGSTTYTPSLKS, | (SEQ ID NO: 54) |
| CDRH3: GGYDVNYF, | (SEQ ID NO: 55) |

and a light chain comprising the following CDRs or
analogs thereof

| | |
|---|---|
| CDRL1: LASQTIGAWLA, | (SEQ ID NO: 56) |
| CDRL2: AATRLAD, | (SEQ ID NO: 57) |
| and | |
| CDRL3: QQFFSTPWT; | (SEQ ID NO: 58) |
| or | |

D: a heavy chain comprising the following CDRs or
analogs thereof

| | |
|---|---|
| CDRH1: GYTMN, | (SEQ ID NO: 73) |
| CDRH2: LINPYNGVTSYNQKFK, | (SEQ ID NO: 74) |
| CDRH3: GDGNYWYF, | (SEQ ID NO: 75) |

and a light chain comprising the following CDRs or
analogs thereof

| | |
|---|---|
| CDRL1: SASSSVTYMHW, | (SEQ ID NO: 76) |
| CDRL2: EISKLAS, | (SEQ ID NO: 77) |
| and | |
| CDRL3: QEWNYPYTF. | (SEQ ID NO: 78) |

31. The isolated binding protein as claimed in claim 29 or 30, which is an isolated humanized or chimeric antibody.

32. An antibody or antigen-binding fragment thereof which specifically binds to CD127, wherein the antibody or fragment of an antibody competes for binding to human CD127 with one or more antibodies selected from the group consisting of:
a. an antibody having the following heavy chain
variable region:
(SEQ ID NO: 29)
EVKLLESGGGLVQPGGSLKLSCAASGFAFSAYWMSWVRQAPGKGLEWIGE
INPDSSTINCTPSLKDKFIISRDNAKNTLSLQMNKVRSEDTALYYCARRL
RPFWYFDVWGAGTTVTVSS,
and the following light chain variable region:
(SEQ ID NO: 30)
DVLMTQTPLSLPVSLGDQASISCRSSQSIVQSNGNTYLEWYLQKPGQSPK
LLIYKVSNRFSGVPDRFSGSGSGTDFTLKISRVEAEDLGVYYCFQGSHVP
RTFGGGTKLEIK;
b. an antibody having the following heavy chain
variable region:
(SEQ ID NO: 51)
DVQLQESGPGLVKPSQSLSLTCTVTGYSITTDYAWNWIRQFPGNKLEWMG
YIFYSGSTTYTPSLKSRISITRDTSKNQFFLQLNSVTTEDTATYYCARGG
YDVNYFDYWGQGTTLTVSS,
and the following light chain variable region:
(SEQ ID NO: 52)
DIQMTQSPASQSASLGESVTITCLASQTIGAWLAWYQQKPGKSPQLLIYA
ATRLADGVPSRFSGSGSGTKFSFKISSLQAEDFVSYYCQQFFSTPWTFGG
GTKLEIK;
c. an antibody having the following heavy chain
variable region:
(SEQ ID NO: 71)
EVQLQQSGPELLKPGASMKISCKASGYSFTGYTMNWVKQSHGKNLEWIGL
INPYNGVTSYNQKFKGKATLTVAKSSSTAYMELLSLTSEDSAVYYCARGD
GNYWYFDVWGAGTTVTVSS,
and the following light chain variable region:
(SEQ ID NO: 72)
EIVLTQSPAITAASLGQKVTITCSASSSVTYMHWYQQKSGTSPKPWIYEI
SKLASGVPVRFSGSGSGTSYSLTISSMEAEDAAIYYCQEWNYPYTFGGGT
TKLEIK;
and
d. an antibody having the following heavy chain
variable region:
(SEQ ID NO: 90)
EVQLQQSGPELVKPGASMKISCKASGYSFTGYTMNWVKQSHGKNLEWIGL
INPYSGITSYNQNFKGKATLTVDKSSSTAYMELLNLTSEDSAVYYCARGD
GNYWYFDVWGAGTTVTVSS,
and the following light chain variable region:
(SEQ ID NO: 91)
EIILTQSPAITAASLGQKVTITCSASSSVSYMHWYQQKSGTSPKPWIYEI
SKLASGVPARFSGSGSGTSYSLTISSMEAEDAAIYYCQYWNYPYTFGGGT
KLEIK;
wherein the antibody is not R34.34 (Dendritics
Inc. #DDX0700).

33. A human, humanised or chimeric antibody or an antigen binding fragment and/or derivative thereof which binds to CD127 and which comprises:
a heavy chain comprising the following CDRs or
analogs thereof

| | |
|---|---|
| CDRH1: GYTMN | (SEQ ID NO: 92) |
| CDRH2: LINPYSGITSYNQNFK | (SEQ ID NO: 93) |
| CDRH3: GDGNYWYF | (SEQ ID NO: 94) |

a light chain comprising the following CDRs or
analogs thereof

| | |
|---|---|
| CDRL1: SASSSVSYMHW | (SEQ ID NO: 95) |
| CDRL2: EISKLAS | (SEQ ID NO: 96) |
| and | |
| CDRL3: QYWNYPYTF. | (SEQ ID NO: 97) |

34. A method for treating an autoimmune disease or inflammatory condition, comprising administering to a patient who is suffering from the autoimmune disease or inflammatory condition a binding protein, antibody or fragment thereof according to any of claims 26 to 33.

35. An antagonist of IL-7 receptor mediated T_{H}17 expansion and/or survival for the treatment of an autoimmune disease or inflammatory disorder in a human subject.

36. An isolated binding protein, antibody or fragment thereof according to any of claims 26 to 33, for the treatment of an autoimmune or inflammatory condition.

37. A method for identifying antibodies suitable for use in the treatment of an autoimmune disease or an inflammatory disease, the method comprising the steps of:
screening a plurality of independent antibody populations to determine the ability of each antibody population to:
i. inhibit the binding of IL-7 to IL-7R,
ii. neutralise IL-7 induced STAT-5 phosphorylation, and/or
iii. inhibit the production of IL-17 by T_{H}17 cells,
and selecting those antibody populations which are able to inhibit the binding of IL-7 to IL-7R, inhibit IL-7 induced STAT-5 phosphorylation, and/or inhibit the production of IL-17 by T_{H}17 cells.
